(19) United States
(12) Patent Application Publication
(10) Pub. No.: US 2018/0158555 A1
(43) Pub. Date: Jun. 7, 2018

(54) MEDICAL KIOSK AND METHOD OF USE

(71) Applicant: Rite Aid Hdqtrs. Corp., Camp Hill, PA (US)

(72) Inventors: Steve Cashman, Powell, OH (US); Kathy Mullins, Dublin, OH (US); Brian Hipsher, Galena, OH (US); John W. Spirk, Gates Mills, OH (US); Jason G. Tilk, Cleveland Heights, OH (US); John R. Nottingham, Bratenahl, OH (US); Jeffrey Kalman, Cleveland Heights, OH (US)

(21) Appl. No.: 15/872,546

(22) Filed: Jan. 16, 2018

Related U.S. Application Data
(63) Continuation of application No. 14/335,037, filed on Jul. 18, 2014, now abandoned.
Application No. 14/335,037 is a continuation-in-part of application No. 13/760,345, filed on Feb. 6, 2013, now Pat. No. 9,043,217.
Application No. 13/760,345 is a continuation-in-part of application No. 13/314,473, filed on Dec. 8, 2011, now Pat. No. 8,996,392.
Application No. 13/314,473 is a continuation-in-part of application No. 29/403,857, filed on Oct. 12, 2011, now Pat. No. D694909.
(60) Provisional application No. 61/469,851, filed on Mar. 31, 2011.
(60) Provisional application No. 61/541,719, filed on Sep. 30, 2011.
(60) Provisional application No. 61/847,741, filed on Jul. 18, 2013.
(60) Provisional application No. 61/847,745, filed on Jul. 18, 2013.
(60) Provisional application No. 61/847,753, filed on Jul. 18, 2013.
(60) Provisional application No. 61/847,760, filed on Jul. 18, 2013.
(60) Provisional application No. 62/016,193, filed on Jun. 24, 2014.

(51) Int. Cl. G16H 80/00; H04N 7/14; G06Q 50/22; G16H 40/20; E04H 3/08; E04H 1/12; G06Q 10/10

(52) U.S. Cl. CPC G16H 80/00; H04N 7/141; G06Q 50/22; G06Q 10/1095; E04H 3/08; E04H 1/1222; G16H 40/20

(57) ABSTRACT

A medical kiosk designed to provide tele-medicine services, check-in services, and/or prescription services for a user. The medical kiosk can include a user video conferencing system that is designed to enable the user to have a real-time or near real-time tele-conference with a medical provider located remotely from the medical kiosk.

The present invention is a continuation-in-part of Ser. No. 13/760,345 filed Feb. 6, 2013, which in turn is continuation-in-part of U.S. Ser. No. 13/314,473 filed Dec. 8, 2011, which in turn claims priority on U.S. Patent Application Ser. Nos. 61/469,851 filed Mar. 31, 2011 and 61/541,719 filed Sep. 30, 2011, which are both incorporated herein by reference.

The present invention is a continuation-in-part of Ser. No. 13/760,345 filed Feb. 6, 2013, which in turn is continuation-in-part of U.S. Ser. No. 13/314,473 filed Dec. 8, 2011, which in turn is a continuation-in-part of United States Design patent application Ser. No. 29/403,857 filed Oct. 12, 2011 (now U.S. Design No. D694,909; issued Dec. 3, 2013), which are both incorporated herein by reference.

The present invention claims priority on U.S. Patent Application Ser. Nos. 61/847,741 filed Jul. 18, 2013; 61/847,745 filed Jul. 18, 2013; 61/847,753 filed Jul. 18, 2013; 61/847,760 filed Jul. 18, 2013, and 62/016,193 filed Jun. 24, 2014, which are all incorporated herein by reference.

The present invention is directed to medical services, more particularly to a method and device for providing medical services to individuals, even more particularly to a method and device for providing medical services to individuals at locations that traditionally have not provided medical services, and still even more particularly to a medical kiosk and method for using a medical kiosk for providing medical services to individuals at locations that are remote from a medical provider.

### BACKGROUND OF THE INVENTION

Medical services are traditionally provided to individuals at a doctor's office or medical facility. Typically, an individual contacts his/her medical provider when the individual requires some type of medical assistance. The medical provider then sets an appointment time and date for the individual to see the medical provider. Many times, the time and date of the appointment are inconvenient for the individual. Furthermore, the individual seeking medical assistance may desire or need more immediate medical assistance and cannot wait for the time and date set by the medical provider. In such situations, the individual goes to the emergency room of a hospital or some type of medical clinic (e.g., Minute Clinic, Take Care Clinic, Urgent Care Clinic, etc.), assuming that such clinics are available or convenient to visit. In other situations, especially in smaller towns and rural areas, a medical facility may be located many miles away thus making a visit to such a facility very inconvenient. As such, many individuals who should seek medical care decide to not seek the advice of medical personnel due to the inconvenience of having to travel large distances to a medical facility.

The costs associated with visiting a medical provider can be costly depending on the type of insurance, if any, the individual carries. When an individual visits the emergency room of a hospital, the medical costs can be substantially higher and insurance coverage may be limited to various types of visits. Insurance coverage and the cost of the visit may also vary at various clinics. In many communities, clinics are not readily available, thus the individual must either visit the medical provider or go to the hospital.

Various pharmacy and drug stores have begun offering medical services on their premises. These locations generally offer flu shots and very basic medical services, and are typically provided by a nurse practitioner, not a doctor. As such, only very limited types of medical services are offered at such locations. Also, these locations are not offered in a private environment. Generally, the services are provided in a side corridor or partitioned location in the facility.

In view of the current state of the medical services, there is a need for providing medical services in a more convenient, desirable, timely and cost effective manner.

### SUMMARY OF THE INVENTION

The present invention is directed to a method and apparatus for providing medical services, diagnoses, health, and/or wellness advice to individuals in a convenient, desirable, timely and cost effective manner. The novel medical apparatus of the present invention and the novel method for providing medical services, diagnoses, health advice, and/or wellness advice addresses the current deficiencies that exist for providing medical services to individuals.

In one non-limiting aspect of the invention, there is provided a method and apparatus for providing medical services, diagnoses, health, and/or wellness advice to individuals that includes a remote medical service arrangement wherein a patient can receive various types of medical advice and services remotely from one or more medical providers (e.g., doctor, nurse practitioner, nurse, psychologist, psychiatrist, optometrist, physician assistant, pharmacist, health coach, dietitian, medical assistant, etc.). Traditionally, a patient was required to go to a medical facility (e.g., hospital, medical clinic, doctor's office, etc.) to personally meet with and be diagnosed by the medical provider. The present invention is directed to a method wherein medical services, diagnoses, health advice, and/or wellness advice can be dispensed by a medical provider at a location that is remote from the patient. The present invention can be used to provide services in one or more different medical specialties (e.g., Allergology; Andrology; Anesthesia; Angiology; Athletic training; Aviation medicine; Cardiology; Dentistry; Dermatology; Disaster medicine; Emergency medicine; Endocrinology; Family medicine; Gastroenterology; General practice; Medical genetics; Geriatrics; Gerontology; Gynaecology; Hematology; Hepatology; Immunology; Infectious diseases; Intensive care medicine; Internal medicine; Military medicine; Nephrology; Neurology; Nuclear medicine; Obstetrics; Oncology; Ophthalmology; Oral and maxillofacial surgery; Orthopedics; Otolaryngology; Paleopathology; Palliative medicine; Pathology; Pediatrics; Podiatry; Psychiatric specialties; Psychiatry; Pulmonology; Radiology; Rehabilitation medicine; Rheumatology; Serology; Sexual health; Sleep medicine; Space medicine; Sports medicine; Surgery; Toxicology; Transplantation medicine; Tropical medicine; Urology; Wilderness medical emergencies; and/or Wilderness medicine). The method and device of the present invention can be used to provide initial screening, treatment, and/or follow-up treatment for a patient. The method and device of the present invention can be used in many situations as an alternative for a patient visiting a medical facility or a doctor's office. However, it can be appreciated that the device of the present invention could be located in a lobby or special region of a doctor's office or medical facility. In one non-limiting arrangement of the present invention, there is provided an audio and/or video link between one or more medical providers located at one or more locations (e.g., medical provider's office, medical provider's home, hospital, etc.) and the patient is located at some other location (e.g., shopping mall, shopping center, drug store, grocery store, department store, warehouse store, discount retailer, discount department store, truck trailer, mobile office, mobile home, office space location, home, car, train, boat, cruise line, friend's home, etc.) that is remote from the one or more medical providers. In essence, the patient can be at any location in which the patient can obtain telephone, satellite, Wi-Fi and/or internet access. The novel method of the present invention for providing medical services, diagnoses, health advice, and/or wellness advice enables a medical provider to provide medical services, diagnoses, health advice, and/or wellness advice to patients without the patient having to physically visit the medical provider and/or having to go to the medical provider's office or place of work. As can be appreciated, the audio and/or video link that is used by the medical provider can enable the medical provider to provide medical services, diagnoses, health advice, and/or wellness advice at a single remote location or a plurality of remote locations. When the audio and/or video link enables the medical provider to provide medical services, diagnoses, health advice, and/or wellness advice to a plurality of remote locations, a single medical provider and/or a plurality of medical providers can be used to provide medical services, diagnoses, health advice, and/or wellness advice to patients that are located at a variety of different remote locations. When a plurality of medical providers is used, the medical providers can be located at the same or different locations. As can be appreciated, the novel method for providing medical services allows for more flexibility for a patient to obtain medical services, diagnoses, health advice, and/or wellness advice. The site at which the patient obtains the medical services, diagnoses, health advice, and/or wellness advice via the method and device of the present invention can be located in a) non-traditional locations that are more convenient to a patient (e.g., shopping mall, public park, department store, retail store, grocery store, museum, office building, business office, business facility, big box stores, government base or facility, drug stores, boat, airplane, train, home, friend's home, car, patient's workplace, etc.), and/or b) traditional locations (e.g., doctor's office, hospital, urgent care facility, etc.) so as to provide easier and/or more convenient access to such medical services, diagnoses, health advice, and/or wellness advice. The geographic location for the device of the present invention can be in a single residence, a single business building, a single neighborhood, multiple neighborhoods, a single town or city, multiple towns or cities, a single state or province, multiple states or provinces, a single country, multiple countries, space, domestic and/or international waterways, etc. The novel method can also be used to provide medical services, diagnoses, health advice, and/or wellness advice at standard times (e.g., 9 am-5 pm Monday through Friday, etc.) and/or non-standard times (e.g., early morning hours, evening hours, weekend hours, holiday hours, etc.) to enable a patient to obtain medical services, diagnoses, health advice, and/or wellness advice at times that are more convenient and timely to the patient.

In another and/or alternative non-limiting aspect of the invention, the novel method and apparatus for providing medical services, diagnoses, health, and/or wellness advice to individuals can be used to provide a variety of different medical services, diagnoses, health advice, and/or wellness advice. Non-limiting examples of such medical services, diagnoses, health advise, wellness advise and/or medical conditions that can be identified, treated and/or addressed include, but are not limited to, acid reflux; hypertension management; allergies; Athlete's Foot; acne; mental health counseling; wellness counseling; asthma; cold sores; vaccinations; arthritis; bronchitis; impetigo; wellness coaching; weight loss; eating disorders; bladder infections; insect stings; allergic reactions; rashes; hemorrhoids; minor burns; health risk management; migraine headaches; common colds; virus infections; bacterial infections; minor skin infections; chronic disease management; coughs; Poison Oak/Ivy; diarrhea; rashes; diabetes; ringworm; lice; ear infections; sties; flu; fever; gout; headache (minor); pink eye; sinus infections; sore throat; ear infections; cramps; STDs; strep throat; throat infections; feeding problems for newborns; vomiting; teething; gastrointestinal problems; anxiety; depression, formula advice for newborns; concussion; head injuries; bone fractures; sprains; hair loss; alopecia; eye infections; urinary tract infections; constipation; appendicitis; pharyngitis; medication therapy management; acid reflux disease; acne; alcohol abuse; allergies; antisocial personality disorder; attention deficit disorder; altitude sickness; Alzheimer's Disease; andropause; anger management; anorexia nervosa; arthritis; Aspergers Syndrome; asthma; autism; back pain; bad breath (halitosis); baldness; bedwetting; bipolar disorder; bladder cancer; body dysmorphic disorder; bone cancer; brain cancer; breast cancer; brain tumors; brain injury; bronchitis; burns; bursitis; cancer; canker sores; Carpal Tunnel Syndrome; celiac disease; cervical cancer; cholesterol; chronic obstructive pulmonary disease; colon cancer; congestive heart failure; cradle cap; Crohn's Disease; dandruff; deep vein thrombosis; dehydration; depression; diabetes; diaper rash; disabilities; diverticulitis; Down Syndrome; drug abuse; smoking cessation; dysfunctional uterine bleeding; dyslexia; ear infections; ear problems; eating disorders; eczema; endometriosis; enlarged prostate; epilepsy; erectile dysfunction; eye problems; fibromyalgia; fracture; gallbladder disease; gallstones; generalized anxiety disorder; genital herpes; genital warts; glomerulonephritis; gonorrhea; gout; gum diseases; gynecomastia; head lice; headache; hearing loss; heart attacks; heart disease; heartburn; heat stroke; heel pain; hemorrhage; hemorrhoids; hepatitis; herniated discs; hiatal hernia; HIV/AIDS; hives; hyperglycemia; hyperkalemia; hypertension; hyperthyroidism; hypothyroidism; infectious diseases; infectious mononucleosis; influenza; infertility; insulin dependent diabetes mellitus; iron deficiency anemia; irritable bowel syndrome; irritable bowel syndrome; itching; joint pain; juvenile diabetes; juvenile rheumatoid arthritis; kidney diseases; kidney stones; leukemia; liver cancer; lung cancer; Mad Cow Disease; malaria; medication management; melena; memory loss; menopause; mesothelioma; migraine; miscarriages; mucus in stool; multiple personality disorder; multiple sclerosis; muscle cramps; muscle fatigue; muscle pain; nail biting; narcissistic personality disorder; neck pain; obesity; obsessive compulsive disorder; osteoarthritis; osteomyelitis; osteoporosis; ovarian cancer; ovarian cyst; pain; pain management; panic attack; Parkinson's Disease; peripheral artery disease; personality disorders; pervasive developmental disorder; Peyronie's Disease; phobias; pink eye; polio; pneumonia; post nasal drip; post-traumatic stress disorder; premature baby; premenstrual syndrome; prostate cancer; psoriasis; reactive attachment disorder; renal failure; restless legs syndrome; rheumatoid arthritis; rheumatic fever; ringworm; rosacea; rotator cuff; scabies; scars; sciatica; schizophrenia; sexually-transmitted disease; sinus infections; skin cancer; skin rash; sleep apnea; sleep disorders; smallpox; snoring; social anxiety; staph infection; stomach cancer; strep throat; Sudden Infant Death Syndrome; sunburn; syphilis; systemic lupus erythematosus; tennis elbow; termination of pregnancy; testicular cancer; tooth decay; tuberculosis; ulcers; urinary tract infection; varicose veins; vertigo; warts; Williams Syndrome; yeast infection; Yellow Fever, etc. As can be appreciated, other or additional types of medical services and/or health care services can be provided. The medical services and/or health care services that can be provided can include, but are not limited to, 1) providing advice and/or recommendations about a medical condition, 2) diagnosing and/or treating a medical condition, 3) providing referral services for a medical condition, 4) prescribing medicine for a medical condition, 5) periodically monitoring a medical condition, 6) providing follow-up checks for a medical condition, 7) providing routine check-up services, 8) providing advice, counseling, and/or recommendations about medical and/or health matters, 9) providing a course of treatment for a medical condition, 8) providing health counseling, 10) providing health information, 12) providing wellness counseling, and/or 13) providing wellness information. As can be appreciated, other or additional services can be provided to the patient. In essence, any type of medical condition, medical concern, health concern, wellness concern, etc. can be addressed in whole or in part by the novel method and apparatus for providing medical services of the present invention. Hereinafter, these services will be collectively referred to as medical services. As can be appreciated, the type of services provided to a patient will depend on the specific medical condition, medical concern or need, wellness concern or need, and/or health concern or need of the patient. In many instances, the medical provider will be able to diagnose, address, advise, consult and/or treat the specific medical condition or need, medical concern or need, wellness concern or need, health concern or need, etc. of the patient. In some instances, the specific medical condition or need, medical concern or need, wellness concern or need, health concern or need, etc. of the patient may be too complicated and/or too complex to address via an audio and/or video link; thus, the medical provider in such situations may need to refer the patient to a hospital, a doctor's office, counselor, psychiatrist/psychologist, medical specialist, health professional, dietician, rehabilitation facility, or a traditional medical clinic for further counseling, treatment and/or diagnosis, or some other location or professional that can address the patient's needs and/or requirements.

In still another and/or alternative non-limiting aspect of the invention, the novel method and apparatus for providing medical services, diagnoses, health, and/or wellness advice to individuals can include the use of a medical kiosk to enable the patient to conveniently communicate with one or more medical providers. One or more medical kiosks can be used in the present invention. Generally, a plurality of medical kiosks which are located at one or more locations are used in the method of the present invention; however, it can be appreciated that a single medical kiosk can be used in accordance with the present invention. Typically, one or more medical providers provide services to one or more medical kiosks. The size, shape, configuration and look of the medical kiosk are non-limiting. The medical kiosk, when in the form of a personal medical kiosk, may merely be in the form of a tablet, laptop, smart phone, etc., that is being used in any location that the patient desires. The medical kiosk, when in the form of a free standing structure, can be configured in a variety of ways. For example, the medical kiosk can take the form of a room, partition area, etc. that includes laptop computer, desktop computer, tablet, etc. Alternatively, the medical kiosk, when in the form of a free-standing structure, can include an enclosure that is designed to enable a patient to enter the enclosure and to communicate with the medical provider. In one non-limiting embodiment of the invention, the medical kiosk provides a private or semi-private environment for a patient to communicate with one or more medical providers that are located remotely from the medical kiosk. The medical kiosk can include an enclosure that is designed to enable a patient to enter the enclosure and to communicate with the medical provider in a private or semi-private manner while in the enclosure of the medical kiosk. The size, shape and configuration of the enclosure of the medical kiosk are non-limiting. In another and/or alternative non-limiting arrangement, the medical kiosk includes one or more walls that form all or a portion of the sides of the enclosure of the medical kiosk. The enclosure may include one or more doors or entry points to enable a patient to enter and/or exit the enclosure. In another and/or alternative non-limiting arrangement, the medical kiosk typically includes a floor and/or a ceiling. The ceiling, when included, can include a portion that is partially or fully transparent; however, this is not required. In another and/or alternative non-limiting arrangement, the medical kiosk can have a modular configuration to enable the parts of the medical kiosk to be set up in various configurations to enable the medical kiosk to be used in various types of spaces and/or to be set up in various types of configurations. The medical kiosk can be formed of any number of materials (e.g., plastic, foam, metal, wood, composite materials, fiber board, etc.). The one or more walls of the medical kiosk can be designed to be interchangeable to enable the door, when used, to be positioned on various locations on the medical kiosk; however, this is not required. The medical kiosk can include a floor and/or ceiling to provide for increased privacy for the patient when the patient is inside the room, cavity or enclosure of the medical kiosk; however, this is not required. The medical kiosk can include one or more tables, ledges, benches, and/or seats in the interior and/or exterior of the medical kiosk; however, this is not required. Such table, ledge, bench, and/or seat, when used, can be designed to be connected in multiple locations on the exterior and/or interior of the medical kiosk; however, this is not required. The modular configuration of the medical kiosk can be such that it can be easily assembled and/or disassembled so that the medical kiosk can be easily brought into a location and easily set up, and/or be easily removed from a location; however, this is not required.

In yet another and/or alternative non-limiting aspect of the invention, the novel method and apparatus for providing medical services, diagnoses, health, and/or wellness advice to individuals can include the use of a medical kiosk in the form of a personal medical kiosk to enable the patient to conveniently communicate with one or more medical providers. The size, shape, configuration and look of the personal medical kiosk are non-limiting. In one non-limiting embodiment of the invention, the medical kiosk, when in the form of a personal medical kiosk, includes a personal computer interface (e.g., laptop computer, desktop computer, tablet, smart phone, notepads, home PC, smart TVs, and/or entertainment console [e.g., Xbox systems, PS3, PS4, Apple TV, Amazon Fire TV, etc.] etc.) that can be used by a user to create a video connection with a remotely-located medical provider. The personal medical kiosk also includes an interface that enables the personal computer interface to communicate with one or more medical devices that are to be used by the patient. In one non-limiting arrangement, the personal medical kiosk is designed to enable a patient to communicate with the medical provider in a private or semi-private manner (e.g., patient's home, car, friend's home, boat, hotel, etc.).

In still yet another and/or alternative non-limiting aspect of the invention, the novel method and apparatus for providing medical services, diagnoses, health, and/or wellness advice to individuals can include the use of a medical kiosk that includes one or more data input terminals; however, this is not required. The one or more data input terminals can be located on one or more locations on the exterior of the medical kiosk; however, this is not required. Alternatively or additionally, the one or more data input terminals can be located on one or more locations in the interior or enclosure of the medical kiosk; however, this is not required. Alternatively or additionally, the one or more data input terminals can be in the form of a laptop computer, desktop computer, tablet, smart phone, etc. The one or more data input terminals can include a video display to display information regarding identification and/or data entry, a camera and/or video camera used to collect information for identification and/or data entry, a keypad or keyboard for identification and/or data entry, a touch screen for identification and/or data entry, microphone and voice recognition software for identification and/or data entry, fingerprint scanner for identification and/or data entry, retina scanner for identification and/or data entry, and/or face and/or body scanners for identification and/or data entry. As can be appreciated, other or additional devices can be included on the medical kiosk to display and/or obtain information regarding identification and/or data entry. The medical kiosk can be used by the patient to enter/convey basic information about the patient. Such information includes, but is not limited to, a) patient name, b) patient address, c) patient contact information (e.g., home address, work address, phone number, email address, pager number, work number, etc.), d) patient age, e) patient sex, f) patient height, g) patient weight, h) patient medical history, i) current medicines used by patient, j) reason(s) for visit by patient, k) patient current symptoms, l) patient insurance information, m) patient payment information, n) patient's current doctor, o) guardian or patent information, p) next of kin information, q) desired medical provider for visit, r) allergy information, s) information about a prior visit, t) medical records information, and/or u) pharmacy information. As can be appreciated, other or additional information can be inputted/conveyed by the patient.

In another and/or alternative non-limiting aspect of the invention, the novel method and apparatus for providing medical services, diagnoses, health, and/or wellness advice to individuals can include a medial kiosk that can be designed to provide information to the patient prior to and/or during the inputting/conveying of information by the patient to the medical kiosk. In one non-limiting embodiment of the invention, the medical kiosk can include audio and/or visual instructions and/or displays used to provide a) information about the medical kiosk, b) how to use the medial kiosk, c) how to properly input/convey information to the medical kiosk, d) provide instructions and/or interactions with the patient during the inputting/conveying of information by the patient to the medical kiosk, e) the wait time for the patient's use of the medical kiosk, f) a list of patients waiting to use the medical kiosk, g) available medical providers, h) types of medical issues that can be addressed by use of the medical kiosk, i) insurance providers that can be used to partially or fully pay for a visit in the medical kiosk, j) payment options for use of the medical kiosk, k) information regarding when the medical kiosk is available in the future, and/or l) information regarding whether the medical kiosk is in use or is available. In another and/or alternative non-limiting embodiment of the invention, the medical kiosk can include light, sound indicators, and/or digital displays to provide information regarding whether the medical kiosk is in use or is available; however, this is not required. In still another and/or alternative non-limiting embodiment of the invention, the medical kiosk can include a notification system to a patient that the medical kiosk is available or will soon be available; however, this is not required. Such a notification can be sent via email, text, phone, pager, internet, digital display, etc. Such notification system can be useful when the medical kiosk is not currently available to the patient. The patient can input the information into the medical kiosk and then go home, run other errands, etc., and then be later notified when the medical kiosk is available or will soon be available. The medical kiosk and/or notification system can also be used to inform the patient when and/or where other medical kiosks are available; however, this is not required. This service, when available, can be used to inform the patient that a nearby medical kiosk has a shorter wait period or is currently available, thus providing the patient with the option of traveling to another available medical kiosk instead of waiting for the current medical kiosk to become available; however, this is not required. This service, when available, can also be used to inform the patient when a prescription is ready for pickup and/or for conveying prescription information to the patient; however, this is not required. This service, when available, can also be used to inform the patient when a follow-up visit is due and/or scheduled; however, this is not required. As can be appreciated, the notification system can be used for other or additional services.

In still another and/or alternative non-limiting aspect of the invention, the novel method and apparatus for providing medical services, diagnoses, health, and/or wellness advice to individuals can include a scheduling system for use of the medical kiosk. The scheduling system allows a patient to schedule an appointment with a medical provider when the patient uses the medical kiosk. The scheduling system can be used to 1) enable a patient to select a particular medical provider, 2) make an appointment on a medical kiosk for a particular time and place, 3) enable a patient to select a medical provider or type of medical provider based on the particular need of the patient (e.g., select a medical provider that specializes in dermatology to address a rash issue on the patient, select a medical provider that specializes in orthopedics to address a sports injury to the knee, etc.), 4) locate available locations of medical kiosks, 5) enable a patient to enter basic information about the patient, 6) check a past, current or future appointment of the patient regarding use of the medical kiosk, 7) enter a partial or full payment for use of the medical kiosk, 8) enter medical insurance information, etc. The scheduling system can be designed to be accessed by a patient on a data entry device on a medical kiosk, via a computer or phone or smart device, and/or by phone. The scheduling system can optionally be designed to enable a patient to request assistance if there a question regarding the use of the scheduling system.

In yet another and/or alternative non-limiting aspect of the invention, the novel method and apparatus for providing medical services, diagnoses, health, and/or wellness advice to individuals can include one of more software applications (e.g., Patient Registration Application, Attendant Application, Patient Appointment or Registration Application, Medical Provider Application, Administrator Application, Patient Portal, Provider Portal, etc.); however, this is not required. For example, the Patient Appointment or Registration Application can include the screens the patient navigates through during the patient registration process; however, this is not required. These screens can optionally appear on the registration station of the exterior of the medical kiosk. The Attendant Application can include screens through which the medical attendant, when used, navigates during the patient appointment; however, this is not required. These screens can optionally appear on a laptop, computer screen, tablet, smart phone, etc. located with the medical attendant and/or at or near the attendant desk on the exterior of the medical kiosk; however, this is not required. The Patient Appointment or Registration Application can include screens through which the patient navigates during the patient consultation; however, this is not required. These screens can optionally appear on the medical provider screen and/or patient screen in the interior of the medical kiosk. The Provider Application can include the screens through which the medical provider navigates during the patient appointment; however, this is not required. These screens can optionally appear on the medical provider's computer, laptop, tablet, smart phone, etc. at his/her remote location. As can be appreciated, other or additional software and/or hardware application can be used with the medical kiosk of the present invention. The Patient Appointment or Registration Application can allow the patient to select a language to be displayed (e.g., English, Spanish, German, French, Chinese, Japanese, etc.); however, this is not required.

In another and/or alternative non-limiting aspect of the invention, the novel method and apparatus for providing medical services, diagnoses, health, and/or wellness advice to individuals can include the use of a Patient Appointment or Registration Application; however, this is not required. The Patient Appointment or Registration Application can include one or more navigational buttons (e.g., Back, Next, Cancel, Request Assistance, etc.) for use by the patient during the registration process; however, this is not required). For example, the Back button can be designed to allow the patient to return to the previous screen, the Next button can be designed to allow the patient to proceed to the next screen, the Cancel button can be designed to allow the patient to cancel a process and return to the beginning of the process (e.g., patient receives a pop-up box asking them if they are sure they want to cancel and return to the beginning of the process. They may choose “Yes” or “No”. “Yes” returns them to the beginning of the process. “No” returns them to the screen they were on when they hit the Cancel button), and the Request Assistance button can be designed to allow the patient to request assistance from the Attendant (e.g., the patient can receive a pop-up box notifying them that the attendant will be with them in a moment and the attendant also receive a notice that the patient requires assistance. The notice provided to the attendant can optionally be color coded and/or generate a certain sound to indicate the source and/or seriousness of the request by a patient and/or medical provider, etc.). The Patient Appointment or Registration Application can include a process through which the patient identifies themselves as a new and/or returning patient and either schedules an appointment at one or more medical kiosks or registers for an existing appointment at one or more medical kiosks. The medical kiosk can optionally be staffed by a medical assistant or attendant at all times. During the patient registration process, the medical assistant or attendant responsibilities, when used, can include welcoming the patient, offering assistance, validating patient identification, initiating/finalizing the patient consultation, and/or completing the sanitization process. As can be appreciated, the medical assistant or attendant can have other or additional responsibilities (e.g., check operation of medical kiosk, report a malfunction of a medical kiosk, monitor appointment status of medical kiosk, assist the patient in the medical kiosk, reload software, etc.). As can be appreciated, the personal medical kiosk generally does not include a medical assistant and/or attendant. The Patient Appointment or Registration Application can optionally be designed to enable a patient to register and schedule appointments through one or more methods (e.g., on-line registration, calling a registration location, visiting a medical kiosk, etc.). During the patient registration process, the Patient Appointment or Registration Application can include scheduling software that provides a scheduling system for a patient to register with one or more medical kiosks. The scheduling system can be designed to be accessed by a patient on a data entry device on a medical kiosk, via a computer, satellite, phone, smart TV, entertainment systems, or any other device that can access a phone signal, satellite signal and/or the internet. The patient registration system can be designed to 1) schedule a new appointment for a patient, 2) schedule an appointment for a returning patient, and/or 3) welcome a patient that has already scheduled an appointment. In one non-limiting arrangement, the patient registration system generally asks if the patient is a returning patient, a new patient or if the patient requires assistance. The patient registration system generally provides the patient with a privacy policy associated with the use of the medical kiosk; however, this is not required. When a privacy policy is presented to the patient, the patient generally must acknowledge or accept the privacy policy before the patient can proceed further with the registration process; however, this is not required. The patient registration system generally provides the patient with terms of service policy associated with the use of the medical kiosk; however, this is not required. When the terms of service policy is presented to the patient, the patient generally must acknowledge or accept the terms of service policy before the patient can proceed further with the registration process; however, this is not required. The patient registration system generally asks why the patient is using the patient registration system (e.g., Medical kiosk tour, Medical kiosk demo, Medical kiosk payment demo, check-in for a medical visit, etc.). The patient registration system generally asks the patient to enter a password, phone number, pin number, name, email address, address, date of birth, sex, or the like to identify the patient; however, this is not required. The patient registration system generally asks the patient to verify if the patient is at least a certain age (e.g., at least two years old, etc.) before medical services can be provided by the medical kiosk; however, this is not required. The patient registration system generally asks the patient to identify one or more symptoms (e.g., body aches, chest congestion, cough, earache or pain, eye itching or swelling, fatigue, fever, headache, nasal congestion, nausea, pink eye, shortness of breath, skin rash, urinary problems, constipation, wheezing, cramps, pulled muscle, stomach pains, indigestion, gas problems, swelling, etc.) that the patient is experiencing for which the patient is seeking medical assistance; however, this is not required. The patient registration system may optionally ask one or more follow-up questions after the patient enters the one or more symptoms and/or may inform the patient to seek an in-person visit by a medical provider; however, this is not required. The patient registration system may optionally ask the patient to identify one or more known allergies of the patient (e.g., none, amoxicillin, aspirin, bee stings, cosmetics, eggs, fish, hay fever, hives, latex, metals, milk, mold, MSG, nuts, drugs, penicillin, pets, poison ivy, shellfish, soy, sulfa, sun, wheat, other, etc.); however, this is not required. The patient registration system may optionally ask the patient to identify one or more known medical conditions of the patient (e.g., none, alcoholism, arthritis, asthma, breast cancer, chronic ear infections, diabetes, emphysema, enlarged prostate, glaucoma, heart attack, high blood pressure, high cholesterol, liver problems, cancer, skin cancer, stroke, thyroid problems, tuberculosis, other, etc.); however, this is not required. The patient registration system may optionally ask the patient to identify one or more past medical procedures that have been performed on the patient; however, this is not required. The patient registration system may optionally ask the patient to identify one or more medications the patient is taking; however, this is not required. The patient registration system may optionally ask the patient if the patient has medical insurance; however, this is not required. If medical insurance is inquired about and the patient indicates that he/she has medical insurance, the patient registration system may optionally ask the patient to enter in the medical insurance information (e.g., insurer name, ID number, policy number, etc.) and/or to scan the insurance card; however, this is not required. The patient registration system may optionally ask the patient to make a copay based on the medical insurance or to fully pay for the medical visit; however, this is not required. Such payments, if any, can be by cash, check, debit card, credit card, smart device, etc.). For debit and credit cards and smart devices, the medical kiosk may include a card reader or scanner or RFID system, and/or may include a key pad to enter the charge or credit information; however, this is not required. A medical assistant or attendant of the medical kiosk can optionally assist the patient with the payment of services and/or with any other check-in step of the patient registration system; however, this is not required. For example, the patient registration system may include a button or other request arrangement to signal to a medical assistance or attendant, if used, that the patient needs assistance with one or more steps of the registration process; however, this is not required. Once the patient is registered, the patient registration system will list one or more days and/or times that the medical kiosk is available for use by the patient; however, this is not required. The time intervals can be spaced by a certain time period (e.g., every twenty minutes, every thirty minutes, etc.); however, this is not required. The patient registration system can verify that an appointment has been made by the patient once all of the required information and optional payment has been made; however, this is not required. Such verification can be merely displayed on the patient registration system, printed out for the patient, emailed or texted to the patient, or left on a voicemail of the patient; however, this is not required. If the patient is a returning patient, the patient may merely need to enter the ID information to access the information that the patient had previously entered and then optionally update such information; however, this is not required. A previous patient may be asked about the new visit (e.g., follow-up, new symptoms, etc.); however, this is not required. The previous patient may optionally be asked to update insurance information, reenter insurance information, etc. and then optionally make the required copay or full payment. If the returning patient is merely returning to a scheduled appointment, the patient may need to enter the ID information and verify that the patient is present and is checked-in or cancelling the scheduled appointment; however, this is not required. A medical assistant or attendant can optionally provide assistance to a patient during any step of the check-in or registration process or recheck-in process; however, this is not required. The information entered and/or scanned regarding the patient can optionally be saved to a file for current and/or later access by the patient, medical provider, medical assistant, etc.; however, this is not required. Such stored information is generally saved on a database, server, etc. that is located remote to the medical kiosk; however, this is not required.

In still another and/or alternative non-limiting aspect of the invention, the novel method and apparatus for providing medical services, diagnoses, health, and/or wellness advice to individuals can include the use of one or more medical assistants or attendants with the medical kiosk; however, this is not required. Generally, medical assistants or attendants are used with a personal medical kiosk. In one non-limiting embodiment of the invention, the medical kiosk can have one or more attendants assist a patient during the use of the medical kiosk (e.g., assist in check-in procedures, assist check-out procedures, assist in entering/exiting the medical kiosk, answering questions about the medical kiosk, assist the patient about the use of the kiosk, assist the patient during examination of the patient by the medical provider, assist in maintaining privacy/security of a patient while using the medical kiosk, assist user during payment of medical services, assist user in obtaining a prescription, assist in answering general questions about the medical kiosk, assist the remote medical provider during the examination of the patient, assist in using one or more medical devices on the patient, cleaning the medical kiosk, maintaining the systems of the medical kiosk, resetting the medical kiosk for use by another patient, assist in processing of payments and/or insurance information of a patient, monitor proper use of the medical kiosk, etc.); however, this is not required. Such attendant, when used, can be a medical provider or non-medical provider. The assistant may or may not have any formal medical education. The one or more attendants, when used, can also or alternatively clean and/or sanitize various regions of the medical kiosk prior to and/or after being used by a patient and/or set up the medical kiosk for a new user; however, this is not required. For example, prior to and/or after one or more patients have entered the medical kiosk, the one or more attendants can clean/sanitize one or more exterior surfaces and/or regions of the medical kiosk (e.g., medical kiosk door, medical kiosk check-in terminal, medical kiosk desk top, medical kiosk exterior walls, medical kiosk touch screen, medical kiosk monitors, seating/tables in waiting area near medical kiosk, etc.); however, this is not required. In an another and/or additional example, prior to and/or after one or more patients have entered the medical kiosk, the one or more attendants can clean/sanitize one or more interior surfaces of the medical kiosk (e.g., medical kiosk door, medical kiosk floor, medical kiosk bench, medical kiosk chair, medical kiosk user terminal, medical kiosk interior desk top, medical kiosk interior walls, medical kiosk touch screen, medical kiosk monitors, medical kiosk instrument doors, medical devices/instruments used by and/or touched by user when in the medical kiosk, any other surface in the interior of the medical kiosk, etc.); however, this is not required. In still another and/or additional example, prior to and/or after one or more patients have entered the medical kiosk, the one or more attendants can set up the medical kiosk for a user (e.g., clean/sanitize interior surfaces of medical kiosk; clean/sanitize medical devices/instruments used and/or touched by a prior user; reposition medical devices/instruments into device storage areas; replace disposable components on medical devices/instruments; replenish paper in a printer; clear a paper jam in a printer; replace batteries for one or more electronic components; close medical device/instrument compartments doors in the medical kiosk; reset user touch screen for next user in the medical kiosk; fix, repair and/or replace non-operating, damaged or broken medical devices/instruments in the medical kiosk; fix, repair and/or replace electronic components, computers, fans, light bulbs, UV bulbs, UV devices, etc. in the interior and/or exterior of the medical kiosk; refill cleaning and/or sanitizing fluid; etc.); however, this is not required. In yet another and/or additional example, the one or more attendants can be used to assist one or more users in the medical kiosk. Generally, such assistance will occur only after requested by the user in the medical kiosk or by the medical provider that is assisting the user while in the medical kiosk; however, this is not required. For instance, the one or more attendants can assist a user in the medical kiosk if the one or more attendants hear a verbal request from the user, receive notice (e.g., light indicator activated by user, sound indicator activated by user, hear user talking via a speaker to attendant, hear user talking through walls of medical kiosk, receive a notice from the medical provider [e.g., phone call, email, light indicator, etc.], etc.); however, this is not required. The one or more medical assistants can be positioned at a desk or table that is positioned adjacent to or connected to the exterior of the medical kiosk; however, this is not required. The medical assistant can be provided with a computer, tablet, etc. to monitor the appointment for the medical kiosk, to cancel an appointment, to reschedule and appointment, to indicate if a patient missed an appointment or is late for an appointment, validate the ID of a patient, to view appointment information of the medical kiosk, to validate a patient's insurance, to monitor the need for the assistant to assist a patient via patient or medical provider request, to monitor the status of the visit by the patient (e.g., medical kiosk cleaned for patient, the patient has properly checked-in, appointment has started and vitals capture process has begun, vitals capture process completed, video consultation by medical provider with patient has begun, the video consultation with the medical provider has been completed, the medical provider is completing the patient report, the visit by the patient in the medical kiosk is completed, etc.), to assist in insurance and information processing and/or scheduling of a patient, to assist in the payment by the patient for medical services, to print out reports and/or email reports to patient that provide a summary or complete report to the patient regarding the visit to the medical kiosk, to activate the sanitation system of the medical kiosk (e.g., UV sanitation system, etc.), to keep a record of when the kiosk was cleaned/sanitized and/or what was cleaned/sanitized by the assistance (e.g., clean patient counter, clean patient touch screen desk, clean patient bench, clean entry doors, clean thermometer, clean dermascope, clean patient chair, clean interior walls, clean medical doors and handles, clean otoscope, clean pulse oximeter, clean floor, clean scale, clean video monitor, clean exterior of the medical kiosk, clean the medical assistant desk, clean the check-in screen on the exterior of the medical kiosk, clean walk-in mat, clean door tracks, clean UVC panel, clean inside ceiling, clean top of medical kiosk, clean UVC lights, clean medical device compartments, etc.), to enter information about the operation of the medical kiosk, to schedule a service call for the medical kiosk, to order parts or accessories for the medical kiosk, to verify the start and completion of an appointment, to process comment information by a patient, to create and/or send reports regarding the use of the medical kiosk, to escort the patient into and/or out of the medical kiosk, to begin the vitals capture process of the patient, provide information and/or instructions to a patient regarding the medical kiosk and/or regarding the proper use of the medical kiosk, to assist a potential patient in a tour of the medical kiosk, etc.

In yet another and/or alternative non-limiting aspect of the invention, the novel method and apparatus for providing medical services, diagnoses, health, and/or wellness advice to individuals can include the use of an Attendant Application; however, this is not required. Generally, an Attendant Application is not used with a personal medical kiosk. The Attendant Application, when used, includes software and/or hardware that enables the attendant/medical assistant to 1) monitor, modify and/or cancel existing appointments for a medical kiosk, 2) monitor whether a patient requires assistance, 3) monitor and/or assist a patient during registration with the medical kiosk, 4) provide procedures and/or check lists for the medical assistant, and/or 5) provide information on the status of the medical kiosk. As can be appreciated, the Attendant Application can have other and/or additional functions. In one non-limiting arrangement, the initial Attendant's screen displays one or more appointment fields (e.g., time, patient name, medical provider name, status, action, etc.). For example, the time field, when used, can be designed to display the time of upcoming appointments on the medical kiosk; the patient name field, when used, can be designed to display the name of the patients for upcoming appointments on the medical kiosk; the provider name field, when used, can be designed to display the names of the providers for upcoming appointments on the medical kiosk; the status field, when used, can be designed to display the status of upcoming appointments on the medical kiosk (e.g., Ready—The medical kiosk has been sanitized and is ready for the next patient appointment, Checked-In—The patient has been checked-in and their appointment is pending, Pre Consult—The appointment has started and vitals capture process is in progress, In Visit—The vitals capture process has been completed and the video consultation with the medical provider is in progress, Post Consult—The video consultation with the medical provider has been completed and the medical provider is completing the visit, Completed—The appointment has been completed, etc.); and the actions field, when used, can be designed to display the availability of the Attendant functions. The actions field, when used, can be designed to allow for one or more actions (e.g., “X” icon, Clock icon, Printer icon, Emergency Clean-Up icon, etc. The “X” icon, when used, can be design to perform one or more functions such as to cancel an upcoming appointment; however, this is not required. The X icon can also allow entry and/or selection of a reason for cancellation (e.g., No Show—This action cancels the appointment, but maintains the patient record; Can't Make Appointment (Not Rescheduling)—This action cancels the appointment, but maintains the patient record; Patient Changed Mind—This action cancels the appointment, but maintains the patient record; Cancel Cancellation—This action cancels the cancellation; etc.). The Clock icon, when used, can be designed to reschedule an upcoming appointment; however, this is not required. When this icon is selected, entry and/or selection of one or more types of data can be allowed (e.g., input a date and time to reschedule an appointment, etc.); however, this is not required. The Printer icon, when used, can be designed to print a visit summary and/or some other type of information (e.g., prescription, coupons, follow-up visit information, survey and/or survey results, etc.); however, this is not required. This icon can be designed to only appear after an appointment has been completed and/or scheduled; however, this is not required. The Emergency Clean-Up icon, when used, can be designed to allow the medical attendant to associate an emergency clean-up with a patient appointment so as to be used for tracking purposes and/or some other purpose; however, this is not required. Upon selection of the Emergency Clean-Up icon, the medical attendant can be allowed to type a note regarding the emergency clean-up; however, this is not required.

In still yet another and/or alternative non-limiting aspect of the invention, the novel method and apparatus for providing medical services, diagnoses, health, and/or wellness advice to individuals can include the use of an Attendant Application that allows the medical attendant/medical assistant to 1) view appointment information, 2) validate patient IDs, 3) validate patient insurance, 4) reschedule or cancel an appointment, and/or 5) perform the sanitization process for the medical kiosk; however, this is not required. In one non-limiting embodiment of the invention, once the patient is “Checked-In” via the patient registration process, the appointment status changes to “Pending.” At that time, the medical attendant can be required to validate the patient's ID; however, this is not required. For example, the medical attendant can 1) select the patient records (e.g., from the computer screen or monitor, etc.) and 2) select the “Validate Patient ID” button or similar type of button or selection; however, this is not required. If the patient is a new patient, the medical attendant can be prompted to insert and scan the patient's identification for the first time to be saved in the patient's electronic file. If the patient is a returning patient, the medical attendant can be prompted to “Update Patient ID”. If “Update Patient ID” is selected, the medical attendant can be prompted to “Start Scan” of updated patient ID. Once the patient's ID is validated and scanned, the medical attendant may be required to rescan the ID (if image quality is compromised) or save the image in the patient's file; however, this is not required. As can be appreciated, other or additional methods can be used to validate a patient's ID. In one non-limiting arrangement, the patient's identification is validated by use of a valid driver's license, US passport, military ID, and/or photo ID card issued by Federal, State, or Local government. As can be appreciated, other or additional types of IDs can be used. If the patient is under 18 or some other age designating a minor, the patient may be required to be accompanied by a parent or legal guardian; however, this is not required. If the patient is not accompanied by a parent or legal guardian, the patient may not be allowed to proceed with the visit. The medical assistant may be required to inform the under-18 or minor-age patient of certain rights when using the medical kiosk (e.g., It is not necessary for the parent or legal guardian to accompany the under-18 patient into the consultation; Anyone under 18 years of age needs to have parent or legal guardian to use the medical kiosk, etc.); however, this is not required. If the patient is under a certain age (e.g., 2 years old, etc.), the medical attendant may be required to inform the patient, parent and/or guardian that the patient to too young to use the medical kiosk, thus cannot proceed with the visit; however, this is not required. If a new patient indicates that they have insurance or a returning patient indicates their insurance information has changed, the patient may be prompted to see the medical attendant and/or be required to enter the insurance information; however, this is not required. The Attendant Application can prompt the medical attendant for entry of a copay amount and/or to scan the insurance card into the patient record; however, this is not required. As can be appreciated, the patient can enter the insurance information without a medical attendant and/or make a copayment without the medical attendant; however, this is not required. Once the patient's insurance is validated and entered and/or scanned, the medical attendant or patient may be required to rescan the ID (if image quality is compromised) and/or reenter the information if not entered properly or save the image and/or entered insurance information into the patient's file; however, this is not required. If the insurance information is not verified at a first try, the patient or medical attendant can attempt to reverify the insurance information; however, this is not required. Once the patient insurance is saved, the patient can be prompted to “Check in” for the next available appointment or select some appointment time and/or day in the future; however, this is not required. Once the patient is checked in for a visit to the kiosk, the appointment status will change to “Pre Consult” or some other statement on the screen of the Attendant Application; however, this is not required. At that time, the medical attendant may be required to recite a “Pre-Visit Summary” (e.g., “After your appointment is verified, you will step into the medical kiosk, your vitals are captured and you will meet with a medical provider over a video connection. The medical provider will provide the diagnoses and you may receive a prescription. The prescription can be sent to a pharmacy of your choice, etc.); however, this is not required. Once the patient is checked in and after any optional statement is made to the patient, the patient can enter the medical kiosk. The medical attendant may be required to escort the patient into the medical kiosk to initiate the vitals capture and/or consultation; however, this is not required. The medical attendant and/or patient can cause the Provider Application to prompt the medical provider to start the medical visit; however, this is not required. Once the medical provider completes the appointment via the Provider Application, the appointment status on the Attendant Application for a particular patient can be designed to change to “Completed”. Once the patient has completed the medical visit and exited the medical kiosk, the medical attendant may be required to perform a sanitization process; however, this is not required. Such sanitation process can include the medical attendant 1) selecting the patient record on the screen, and 2) selecting the Start Sanitization button or similar labeled button or selection. The medical attendant can enter the medical kiosk and perform the sanitization process. A screen that includes a check list or a paper check list can be used by the medical attendant to check off items that have been sanitized. For example, a screen inside the medical kiosk can include a check list and the medical assistant checks off each item as such item is sanitized by the medical attendant; however, this is not required. When all items are sanitized and optionally checked off, the medical attendant can select a button on the Attendant Application that the sanitation step is completed; however, this is not required. The medical attendant can optionally select to “Run UVC Light” or some other or additional automated sanitation system if such option is available; however, this is not required. Areas of the medical kiosk that can be cleaned after each visit and/or cleaned at period times based on some protocol are: patient counter, patient monitor screen, physician monitor screen, patient chair, seat at rear of station, kiosk wall panels, medical devices, medical device doors and handles, interior of kiosk, entry door of kiosk, internal wood surfaces in kiosk, attendant station, walk in door mat, floor of kiosk, door track of kiosk, external display monitor of kiosk, external surfaces of kiosk, UVC panel in kiosk, and/or UVC lights. The surfaces can be cleaned with wipes (e.g., Virox wipes [Towelettes with disinfecting chemicals that kill 99% of viruses and bacteria], glass cleaner, surface cleaners, floor cleaners, duster, dusting cloth, etc). Upon completion of the sanitization process, the medical kiosk status can return to “Ready” on the Attendant Application; however, this is not required. At that point, the medical attendant can start validation of the next patient's ID.

In another and/or alternative non-limiting aspect of the invention, the novel method and apparatus for providing medical services, diagnoses, health, and/or wellness advice to individuals can include the use of a Patient Appointment or Registration Application to navigate patients through the vitals capture process, patient consultation, and/or survey; however, this is not required. The Patient Appointment or Registration Application can contain one or more navigational buttons on the patient screen that is located inside the medical kiosk (e.g., Back, Next, Cancel, Request Assistance, etc.); however, this is not required. The Back button, when used, allows the patient to return to the previous screen. The Next button, when used, allows the patient to proceed to the next screen. The Cancel button, when used, allows the patient to cancel a process and return to the beginning of the process. The patient can optionally receive a request to confirm the cancel procedure; however, this is not required. The Request Assistance button, when used, allows the patient to request assistance from the medical attendant. The selection of this button can be designed to also notify the patient that the medical attendant will be with them in a moment; however, this is not required. The selection of the Request Assistance button by the patient can result in the Attendant Application displaying on a screen a notice or generating a warning sound, etc. that the patient has requested assistance. For example, when the patient initiates the Request Assistance button, a pop-up box (e.g., orange box, red box, blue box, etc.) can appear on the medical attendant's screen that is labeled “kiosk,” colored orange, and indicates the patient requires assistance. As can be appreciated, the screen of the Provider Application, when used, can include an Alert Attendant button to notify the medical attendant that the patient requires assistance; however, this is not required. If the medical provider initiates the Alert Attendant button from the Provider Application, a pop-up box can be designed to appear on the medical attendant's screen that indicates the patient requires assistance. The pop-up box can be a colored box (e.g., red box, green box, orange box, etc.); however, this is not required. A sound can also or alternatively be generated by the Attendant Application, when used, to notify the medical attendant that the medical provider has selected the Alert Attendant button; however, this is not required. The Patient Appointment or Registration Application, when used, can be designed to allow the patient to 1) capture vitals, 2) perform the provider consultation, and/or 3) perform the survey. As can be appreciated, the Patient Appointment or Registration Application, when used, can be designed to allow the patient to perform other or additional functions. The Patient Appointment or Registration Application can be designed to appear on the Patient Screen and/or Provider Screen that are located in the interior of the medical kiosk; however, this is not required. The Provider Screen, when used, is the screen that is located above or adjacent to the Patient Screen; however, this is not required. The medical provider generally appears on the Provider Screen when the consultation starts; however, this is not required. The Patient Screen, when used, can provide information to the patient while located in the kiosk and/or allow the patient to enter information (e.g., patient receives vitals capture instructions on the Patient Screen and enters information as prompted, etc.). The Patient Appointment or Registration Application can be designed to capture vitals of the patient. The Patient Appointment or Registration Application can be designed to require the patient to initiate the vitals capture procedure and/or the medical attendant can initiated the vitals capture procedure for the patient. The Patient Appointment or Registration Application can be designed to navigate the patient and/or provide instructions to the patient for one or more of the vitals capture procedures; however, this is not required. During the vitals capture process, the patient can request that the medical assistant assist the patient in one or more of the procedures for capturing the vitals; however, this is not required. The Patient Appointment or Registration Application can be designed to require the patient to confirm that instructions for a particular vital capture procedure have been read and/or understood before proceeding with the next vital capture step; however, this is not required. The Patient Appointment or Registration Application can be designed to have the patient enter his/her height on the Provider Screen and/or Patient Screen; however, this is not required. The interior of the medical kiosk can include a height marker or height tape to enable a patient to determine his/her height; however, this is not required. The medical kiosk can include a camera or some other type of arrangement that can be used to automatically determine the height of the patient while the patient is positioned in the medical kiosk; however, this is not required. The Patient Appointment or Registration Application can be designed have the patient enter his/her weight on the Provider Screen and/or Patient Screen; however, this is not required. The interior of the medical kiosk can include a scale (e.g., built-in floor scale, etc.) that can be used by the patient to determine the patient's weight; however, this is not required. When a scale is provided in the medical kiosk, the weight can be automatically transferred to the Patient Appointment or Registration Application and/or can be manually entered by the patient. The Patient Appointment or Registration Application can be designed to have the patient obtain his/her temperature. The Patient Appointment or Registration Application can be designed to open a medical device cabinet that contains a thermometer; however, this is not required. The Patient Appointment or Registration Application can be designed to provide instructions and/or a video on how to use the thermometer (e.g., a) remove thermometer from open medical cabinet, b) place thermometer in ear, c) remove thermometer from ear after hearing a beep sound or other indicator that indicates procedure is completed, d) return thermometer to medical cabinet, etc.); however, this is not required. The Patient Appointment or Registration Application can be designed to have the patient obtain his/her blood pressure. The Patient Appointment or Registration Application can be designed to open a medical device cabinet that contains a blood pressure cuff and/or allows the blood pressure cuff to be plugged into the medical kiosk; however, this is not required. The Patient Appointment or Registration Application can be designed to provide instructions and/or a video on how to use the blood pressure cuff; however, this is not required. As can be appreciated, the medical attendant can place the blood pressure cuff on the patient entering the medical kiosk and/or after the patient has entered the medical kiosk; however, this is not required. If the blood pressure cuff requires that it be connected to the medical kiosk, the medical attendant or the patient can perform such connection. The patient or the medical attendant can start the blood pressure reading by selecting a button on the Patient Appointment or Registration Application; however, this is not required. The blood pressure cuff can be used to measure the blood pressure/heart rate of a patient. A real-time reading of the blood pressure/heart rate of a patient can be displayed on the Patient Screen and/or Provider Screen; however, this is not required. The patient or medical attendant can stop the process of capturing the blood pressure/heart rate of the patient; however, this is not required. After the completion of capturing all the vitals, the Patient Appointment or Registration Application can be designed to display on the Patient Screen and/or Provider Screen a summary of one or more of the captured vitals. The Patient Appointment or Registration Application can be designed to allow a patient to manually change one or more of the collected vitals; however, this is not required. Once the vitals capture process is completed, the patient can be prompted (e.g., “I'm Ready” button, etc.) to indicate that the patient is ready for the conference with the medical provider; however, this is not required. The Patient Appointment or Registration Application can be designed to notify the patient that the medical provider will be with them shortly; however, this is not required. Once the consultation has begun, the Patient Appointment or Registration Application can be designed to cause the medical provider to appear on the lower half of the Provider Screen and the patient's vitals appear on the upper half of the Provider Screen; however, this is not required. As can be appreciated, the full Provider Screen can only include the medical provider. The Patient Appointment or Registration Application can be designed to allow the patient to adjust the volume by use of volume buttons on the Patient Screen; however, this is not required. As can be appreciated, volume control can be located in other or additional locations in the medical kiosk. After the completion of the consultation, the Patient Appointment or Registration Application can be designed to ask the patient to complete a survey; however, this is not required. The Patient Appointment or Registration Application can be designed to allow the patient to skip the survey, when used, or to proceed with the survey; however, this is not required. If the patient selects the survey, when offered, one or more questions can be displayed on the Patient Screen and/or Provider Screen; however, this is not required. As can be appreciated, the medical attendant can provide the patient with a written survey and/or ask verbal survey questions; however, this is not required. At the completion of the survey, or if the patient chooses to skip the survey when such survey is offered, the Patient Appointment or Registration Application can be designed display a final screen thanking the patient for his/her visit and/or prompting them to request a visit summary from the medical attendant; however, this is not required.

In still another and/or alternative non-limiting aspect of the invention, the novel method and apparatus for providing medical services, diagnoses, health, and/or wellness advice to individuals can include the use of a Provider Application that is designed to enable the medical provider to navigate through the appointment process during the patient appointment; however, this is not required. The Provider Application can be designed to allow the medical provider to a) display appointments, b) display patient information, c) view and/or modify visit information, d) perform the consultation, and/or e) utilize one or more medical devices in the medical kiosk. The Provider Application can be designed to cause screens to appear on the medical provider's computer, smart phone, smart TV, tablet, etc. at the medical provider's remote location; however, this is not required. The Provider Application can be designed to enable the medical provider to view appointments that have been completed by the medical provider, that are in progress by the medical provider and/or which are future appointments. In one non-limiting arrangement, the Provider Application can be designed to enable the medical provider to view appointments that have been completed by the medical provider or are in progress for a certain day (e.g., today, last Monday, etc.). In another non-limiting arrangement, the Provider Application can be designed to enable the medical provider to view appointments that have been made for a future time (e.g., two hours from the present, the next day, etc.). For example, the Provider Application can be designed to display on one portion of the screen (e.g., left side, right side, center, etc.) appointments for Today and Future. In one non-limiting arrangement, the Today tab can be designed to display one or more types of patient appointment information (e.g., Time—Time of appointment, Patient Name—Patient Name for appointment, Status—Status of appointment, Pending—Patient started the registration process, but has not yet been approved for payment, Checked-In—Patient has been checked-in, Pre Consult—Appointment has started and vitals are being collected, In Visit—Vitals have been collected and the video consultation is in-progress, Post Consult—Video consultation has been completed and medical provider completion is pending, Completed—Appointment has been completed, etc.). The Future tab can be designed to display one or more types of patient appointment information (e.g., Date—Date of appointment, Time—Time of appointment, Patient Name—Patient Name for appointment, etc.). The Provider Application can be designed to enable the medical provider to view a selected record on another portion of the screen; however, this is not required. The selected record can provide the medical provider one or more types of information (e.g., Patient Name, Patient Date of Birth, Patient Sex, Symptoms, Medical Conditions, Date of Last Visit, Diagnosis, Allergies, Medications, Prior Visit Record of Patient, Medical Records of Patient, etc.). The Provider Application can be designed to enable the medical provider to notify the medical attendant that the patient requires assistance; however, this is not required. The Provider Application can be designed to enable the medical provider to view the appointment and/or patient information prior to the vitals capture process. The Provider Application can be designed to limit access to a patient's information until a certain point (e.g., patient status is Checked-In, etc.); however, this is not required. The Provider Application can be designed to enable the medical provider to refuse an appointment that has been created for the medical provider; however, this is not required. Generally, such refusal should occur prior to the patient status of Checked-In to enable the system to locate another available medical provider; however, this is not required. The Provider Application can be designed to allow the medical provider to begin the conference with the patient once the patient is ready in the medical kiosk (e.g., after Check-In, after Capture of Vitals, etc.); however, this is not required. The Provider Application can be designed to cause a Start Visit button or similar button to appear on the medical provider's screen to allow the medical provider to begin the consultation with the patient; however, this is not required. Once the visit or consultation has been initiated, the Provider Application can be designed to update the appointment status to In Visit; however, this is not required. Once the visit or consultation has begun, the patient appears on a portion of the medical provider's screen (e.g., right side of screen, center of screen, left side of screen, etc.); however, this is not required. The medical provider may greet the patient in the medical kiosk with a greeting (e.g., “Welcome. Can you see me ok? Can you hear me ok?”, etc.); however, this is not required. The Provider Application can be designed to cause an End Consultation or similar button to appear on the medical provider's screen to terminate the consultation or visit with the patient and thereby terminate the video link between the medical provider and patient in the medical kiosk; however, this is not required. Once the consultation or visit with the patient is terminated, the Provider Application can be designed to cause the status of the patient to change to Post Consult; however, this is not required Post Consult means the video consultation has been completed and the medical provider is completing the visit summary. Once the medical provider has completed the visit summary and written any required prescriptions, the Provider Application can be designed to cause a Complete Appointment or similar button to appear on the medical provider's screen; however, this is not required. The selection of the Complete Appointment button or similar button after completion of the video consultation can result in the status of the patient to change to Completed; however, this is not required. Completed means the medical provider has completed input of all information and is ready to commence the next appointment. The Provider Application can be designed to allow the medical provider to view the patient records, including the vitals capture process that has occurred or is occurring in the medical kiosk. The Provider Application can be designed to cause one or more tabs to be displayed on the medical provider's screen once a patient record has been selected (e.g., Patient Information, Current Visit, Previous Visit, etc.). The Patient Information tab, when used, can list one or more types of information of the patient on the medical provider's screen (e.g., Personal Information, Medications, Allergies, Medical Conditions, etc.). One or more of these categories of information can be further expanded upon selection by the medical provider; however, this is not required. For example, selection of the Personal Information category can cause further information about the patient to be displayed (e.g., date of birth, sex, alcohol usage, smoker status, allergies, patient personality traits, marriage status, medication usage, drug history, psychological profile, recent medical/psychological issues, medical/drug compliance, profile, race, eye color, native language, citizenship, address, etc.); however, this is not required. The Current Visit tab, when used, can list one or more types of information about the patient on the medical provider's screen (e.g., Medications, Notes, Vitals, Devices, Visit Summary, Attendant Instructions, etc.). One or more of these categories of information can be further expanded upon selection by the medical provider; however, this is not required. For example, selection of the Medications category can cause further information about medications used by the patient (e.g., past medications used, past medications prescribed to patient, current medications used by the patient, medication compliance, etc.). The Previous Visit tab, when used, can list one or more types of information of the patient on the medical provider's screen (e.g., Symptoms, Notes, Vitals, Visit Summary, etc.). One or more of these categories of information can be further expanded upon selection by the medical provider; however, this is not required. For example, selection of the Vitals category can cause further information about the vitals that were captured during a previous visit to the current medical kiosk, some other medical kiosk, or at some other medical facility. In one non-limiting embodiment of the invention, the Provider Application is designed to cause at least three tabs to be displayed on the medical provider's screen once a patient record has been selected, namely Patient Information, Current Visit, and Previous Visit. The Patient Information tab contains sections for Personal Information, Medications, Allergies, and Medical Conditions; however, it can be appreciated that other or additional tab sections can be included. The Personal Information section contains one or more sub-sections, of which some or all the information was entered by the patient during the registration process. This information generally cannot be modified by the medical provider; however, this is not required. Non-limiting examples of information that can be displayed in the Personal Information section includes Date of Birth, Gender—Male or Female, Alcohol Use, Smoker status, Height, Age, Contact Information, Race, Eye Color, Hair Color, Native Language, Citizenship, Address, Psychological profile, etc. The Medications section allows the medical provider to see previously entered medications, search and select new medications, remove medications the patient is no longer taking, view medication compliance history of patient, etc. The Provider Application can be designed to allow a medical provider to add a medication by having the medical provider type in the medication and/or to start typing the medication name in the Search Medications drop down and allow a listing of matching medication names to appear for selection and then to select the proper medication. The Provider Application can be designed to allow a medical provider to delete a medication by having the medical provider select an X next to the medication. As can be appreciated, other or additional arrangements can be used to add and/or delete medications in a patient's record. The Allergies section allows the medical provider to view allergies as input by the patient. This function allows the medical provider to see previously entered allergies, search and select new allergies, and/or remove allergies. The Provider Application can be designed to allow a medical provider to add an allergy by having the medical provider type in the allergy and/or to start typing the allergy name in the Add Allergy dropdown and allow a listing of matching allergy names to appear for selection and then to select the proper allergy. The Provider Application can be designed to allow a medical provider to delete an allergy by having the medical provider select an “X” next to the allergy. As can be appreciated, other or additional arrangements can be used to add and/or delete allergies in a patient's record. The Medical Conditions section allows the medical provider to view patient medical conditions as input by the patient. This function allows the medical provider to see previously entered medical conditions, search and select new medical conditions, and/or remove medical conditions. The Provider Application can be designed to allow a medical provider to add a medical condition by having the medical provider type in the medical condition and/or to start typing the medical condition in the Add Medical Condition dropdown and allow a listing of matching medical condition names to appear for selection and then to select the proper medical condition. The Provider Application can be designed to allow a medical provider to delete a medical condition by having the medical provider select an X next to the medical condition. As can be appreciated, other or additional arrangements can be used to add and/or delete medical conditions in a patient's record. The Current Visit tab contains sections for Symptoms, Notes, Vitals, Devices, Visit Summary, and Attendant; however, it can be appreciated that other or additional tab sections can be included. The Symptoms section contains all of the patient's symptoms for the current visit as input by the patient. This function allows the medical provider to see entered symptoms, search and select new symptoms, and remove symptoms. The Provider Application can be designed to allow a medical provider to add a symptom by having the medical provider type in the symptom and/or to start typing the symptom in the Symptoms dropdown and allow a listing of matching symptom names to appear for selection and then to select the proper symptom. The Provider Application can be designed to allow a medical provider to delete a symptom by having the medical provider select an “X” next to the symptom. As can be appreciated, other or additional arrangements can be used to add and/or delete symptoms in a patient's record. The Notes section allows the medical provider to enter notes for the current visit. This information generally does not appear on the Appointment Summary and is intended for internal reference only; however, this is not required. The Provider Application can be designed to allow a medical provider to add or delete a note by typing a note in the note texted box or by deleting a note in the note text box. As can be appreciated, other or additional arrangements can be used to add and/or delete notes in a patient's record. The Vitals section contains the patient's vitals for the current visit. They are displayed dynamically on the provider's screen as the vitals capture process takes place; however, this is not required. This function allows the medical provider to see the vitals prior to starting and during the consultation; however, this is not required. This information is generally only displayed and cannot be updated; however, this is not required. Non-limiting vitals can include Height, Weight, Blood Pressure, Heart Rate, Oxygen Saturation, and Temperature. The Devices section contains icons of the medical devices that are located in the medical kiosk and/or can be used with the medical kiosk. On the interior of the medical kiosk, there can optionally be one or more medical containers/cabinets (e.g., 1-10, 2, 4, 6, etc.), wherein one or more can be locked upon patient entry into the medical kiosk. As can be appreciated, personal medical kiosks and other types of medical kiosks may or may not include and/or container/cabinets. The medical provider has the capability to unlock one or more of the medical cabinets in the medical kiosk as necessary. When the medical provider wants to unlock a medical device cabinet, the medical provider clicks on or selects the appropriate medical device icon on the medical provider's computer screen. This selection action can result in the activation of the medical device and/or the opening of the medical cabinet door that includes the selected medical device. As can be appreciated, the medical device cabinet can be opened by other or additional means. An audible sound can be generated (e.g., sound of dropping door, sound of a lock unlocking, etc.) when access to a medical cabinet is granted to a patient; however, this is not required. As can be appreciated, a visual indicator can also or alternatively be used to indicate that a patient can access a particular medical cabinet; however, this is not required. For personal medical kiosks, medical cabinets are not typically used. For personal medical kiosks, the medical provider generally informs the patient to use a particular medical device. Non-limiting medical devices that can be used during the use of the medical kiosk can include a thermometer, otoscope, stethoscope, dermascope, pulse oximeter, spirometer, blood pressure cuff, blood sugar analyzer, Blood Pressure Cuff, blood analyzer, etc. As can be appreciated, the medical attendant, when used, can provide one or more medical devices to a patient prior to and/or during the visit (e.g., blood pressure cuff, etc.). The Visit Summary section can be comprised of one or more sub-sections, some or all of which are input by the medical provider (e.g., Current Diagnoses, Treatment Plan, Follow-Up Care, Visit Documents, etc.); however, this is not required. The Current Diagnoses section, when used, allows for the provider to input diagnoses codes and descriptions for the current visit. The medical provider can update and/or enter a diagnoses by entering the name of the diagnoses and/or an ICD code in a drop down menu and/or search menu. Once the diagnoses name has been entered and/or searched and located, the medical provider can add the diagnoses to the patient's record. More than one diagnoses can be added to a patient's record during a single visit. Once the one or more diagnoses are entered, such diagnoses can be optionally displayed under the Current Diagnoses section. The Treatment Plan section, when used, allows for the medical provider to input a treatment plan for the current visit. Such treatment plan can be typed by the medical provider into a text box and this information can appear on the Appointment Summary for the patient to reference; however, this is not required. The Follow-Up Care section, when used, allows for the medical provider to input follow-up care for the current visit. Such follow-up care plan can be typed by the medical provider into a text box; however, this is not required. This information can appear on the Appointment Summary for the patient to reference; however, this is not required. The Visit Document section, when used, allows for the medical provider to input follow-up care for the current visit. The medical provider can include one or more visit documents and/or other types of documents (e.g., documents about the common cold, documents about rashes, documents about the flu, documents about lice, documents about acne, documents about abstinence/birth control, documents about drug usage/abuse, documents about alcohol usage/abuse, documents about child care, documents about personal hygiene, etc.) which can be provided to the patient along with the Appointment Summary; however, this is not required. The Attendant Instructions section, when used, allows for the medical provider to input attendant instructions for the current visit. Such information can be entered into a text box by the medical provider. This information may or may not be provided to the patient. The Previous Visit tab contains information about prior medical visits by the patient. When the Previous Visit tab is selected, a dropdown appears containing the dates of all previous visits. The medical provider can select a previous visit and then review information about the previous visit. Typically, the medical provider can only view one previous visit at a time. In one non-limiting arrangement, when the previous visit date is selected, one or more sections are displayed (e.g., Symptoms, Notes, Vitals, Visit summary, etc.). The Symptoms section, when used, allows for the medical provider to view all of the patient's symptoms from the previous visit as input by the patient and/or medical provider. The Notes section, when used, allows the medical provider to view all notes for the previous visit that were input by the medical provider in the prior visit. The Vitals section, when used, allows for the medical provider to view all of the patient's vitals that were captured during previous visits. The Visit Summary section, when used, allows for the medical provider to view all visit summary information input during a prior visit. This section can include one or more subsections (e.g., Treatment Plan, Follow-up Care, Visit Documents, etc.).

In yet another and/or alternative non-limiting aspect of the invention, the novel method and apparatus for providing medical services, diagnoses, health, and/or wellness advice to individuals can include a method and a kiosk wherein before the video-conference between the medical provider and the patient begins, each patient starts with a Vitals Check to capture the following one or more of the information: Height, Weight, Temperature, Blood Pressure and/or Heart Rate. Thereafter, the patient and/or medical assistant can inform the medical provider that the visit is ready to begin; however, this is not required.

In still yet another and/or alternative non-limiting aspect of the invention, the novel method and apparatus for providing medical services, diagnoses, health, and/or wellness advice to individuals can include a method and a kiosk wherein the kiosk includes one or more (e.g., six, etc.) advanced medical device cabinets which are locked upon patient entry; however, this is not required. Generally, the personal medical kiosk does not include the use of medical device cabinets. As can be appreciated, other types of kiosks may not include medical device cabinets. During the patient consultation, the medical provider has the capability to unlock and open these cabinets from the Provider Application on the Provider's computer; however, this is not required. The following medical devices can be contained within the cabinets: a thermometer, an otoscope, a stethoscope, a dermascope, a pulse oximeter, blood analyzer, and/or a blood pressure cuff Other or additional medical devices can be included in the medical kiosk (e.g., scale, height tape, eye chart, blood sugar analyzer, defibrillator, etc.). As can be appreciated, these medical devices can also be used with the personal medical kiosk. The stethoscope, when used, is used for detecting sounds produced in the body that are conveyed to the ears of the listener. The medical kiosk stethoscope sounds are transmitted from the stethoscope in the medical device (e.g., hardwire, USB, Bluetooth, Wi-Fi, etc.) to the medical provider's stethoscope. On the interior of the medical kiosk, the stethoscope can be contained in the first medical device cabinet on the left, when used; however, this is not required. The stethoscope can be activated by the medical provider via the Provider Application by the medical provider selecting the Stethoscope icon the provider's screen; however, the stethoscope can be activated by other or additional arrangements or always remain active. In one non-limiting arrangement, when the medical provider via the Provider Application selects the stethoscope icon on the provider's screen, the cabinet door in the medical kiosk for the stethoscope opens (e.g., drops down, etc.) to enable the patient in the medical kiosk to access the stethoscope; however, this is not required. The medical provider can provide instructions to the patient on how to use the stethoscope (e.g., Once the patient has removed the device, request for patient to turn on the stethoscope on the stethoscope device. The “On” button is the lower most button on the device; it looks like a smiley face with one eye. Tell the patient to attempt to hold the device on the sides, while using it, so other buttons are not depressed, etc.) and/or instructions for use of the stethoscope can be provided on the Patient Screen and/or Provider Screen in the medical kiosk; however, this is not required. When the stethoscope is selected, a video image can be designed to appear on the Provider Application (e.g., medical provider's screen) and/or the Provider Screen and/or Patient Screen that is located in the interior of the medical kiosk. If a video image option is available for display on the Provider Screen and/or Patient Screen that is located in the interior of the medical kiosk, the medical provider via the Provider Application can turn off such video image on the Provider Screen and/or Patient Screen; however, this is not required. The medical provider via the Provider Application can capture images and/or data generated by the stethoscope and/or video camera in the medial kiosk for placement in the patient's file; however, this is not required. The medical provider via the Provider Application can optionally annotate an image once captured (e.g., use the paint brush and font icons to modify image, etc.) and/or show the captured image to the patient; however, this is not required. The medical provider via the Provider Application can increase and/or decrease the volume received from the stethoscope; however, this is not required. As can be appreciated, the patient can increase and/or decrease the volume from the stethoscope; however, this is not required. The medical provider and/or patient can request assistance by the medical assistant, when available, related to the stethoscope if so required. The otoscope, when used, is a device used for examining the internal ear. The image generated by the otoscope in the medical kiosk can be displayed on the Patient's Screen and/or Provider's Screen in the medical kiosk and/or on the screen being used by the medical provider; however, this is not required. The transmission of the signal can be by various means (e.g., hardwire, USB, Bluetooth, Wi-Fi, etc.). On the interior of the medical kiosk, the otoscope can be contained in the second medical device cabinet on the left in the medical kiosk, when a cabinet is used; however, this is not required. The otoscope can be activated by the medical provider via the Provider Application by the medical provider selecting the otoscope icon on the provider's screen; however, the otoscope can be activated by other or additional arrangements or always be on. In one non-limiting arrangement, when the medical provider via the Provider Application selects the otoscope icon on the provider's screen, the cabinet door in the medical kiosk for the otoscope opens (e.g., drops down, etc.) to enable the patient in the medical kiosk to access the otoscope; however, this is not required. The medical provider can provide instructions to the patient on how to use the otoscope (e.g., Once the patient has pulled out the device, request for patient to place the otoscope in the appropriate ear lobe, then adjust the focus dial. The focus dial is on the top of the otoscope. The patient can push it to the top and then dial it down to determine to focus; To direct the patient to maneuver the otoscope for best image capture, use the interior of the medical kiosk as reference points. Once the device is in the patient's ear canal, instruct them (or the individual assisting them) to move the device towards the front, back, bottom, and top of the medical kiosk, etc.) and/or instructions for use of the otoscope can be provided on the Patient Screen and/or Provider Screen in the medical kiosk; however, this is not required. When the otoscope is selected, a video image can be designed to appear on the Provider Application (e.g., medical provider's screen) and/or the Provider Screen and/or Patient Screen that is located in the interior of the medical kiosk. If a video image option is available for display on the Provider Screen and/or Patient Screen that is located in the interior of the medical kiosk, the medical provider via the Provider Application can turn off such video image on the Provider Screen and/or Patient Screen; however, this is not required. The medical provider via the Provider Application can capture images and/or data generated by the otoscope and/or video camera in the medial kiosk for placement in the patient's file; however, this is not required. The medical provider via the Provider Application can optionally annotate an image once captured (e.g., use the paint brush and font icons to modify image, etc.) and/or show the captured image to the patient; however, this is not required. The medical provider via the Provider Application can adjust the focus of the otoscope; however, this is not required. As can be appreciated, the patient can adjust the focus of the otoscope; however, this is not required. The medical provider and/or patient can request assistance, when available, by the medical assistant related to the otoscope if so required. The thermometer, when used, is used for detecting human internal heat. The temperature that is transmitted by the thermometer can be displayed on the Patient's Screen and/or Provider's Screen in the medical kiosk and/or on the screen being used by the medical provider; however, this is not required. The transmission of the signal can be by various means (e.g., hardwire, USB, Bluetooth, Wi-Fi, etc.). On the interior of the medical kiosk, the thermometer can be contained in the third medical device cabinet on the left in the medical kiosk, when a cabinet is used; however, this is not required. The patient's temperature can be collected during the vitals capture process which is prior to the consultation with the medical provider; however, this is not required. If the medical provider wants the temperature to be retaken or if the temperature of the patient has not already been taken, the thermometer can be activated or reactivated by the medical provider via the Provider Application by the medical provider selecting the thermometer icon on the provider's screen; however, the thermometer can be activated by other or additional arrangements or always be on. In one non-limiting arrangement, when the medical provider via the Provider Application selects the thermometer icon on the provider's screen, the cabinet door in the medical kiosk for the thermometer opens (e.g., drops down, etc.), if not already open, to enable the patient in the medical kiosk to access the thermometer; however, this is not required. The medical provider can provide instructions to the patient on how to use the thermometer (e.g., Once the patient has pulled out the device, request for patient to turn on the thermometer. The “On” button is on the inside handle of the device; Request for the patient to insert the thermometer into their ear and press the button on top of the handle to start the reading. Once the patient's temperature is collected, a beep will sound; Once the beep is sounded to indicate the temperature reading has completed, the patient can hit the “Temperature Recorded” button on the patient screen, etc.) and/or instructions for use of the thermometer can be provided on the Patient Screen and/or Provider Screen in the medical kiosk; however, this is not required. When the thermometer is selected, a video image can be designed to appear on the Provider Application (e.g., medical provider's screen) and/or the Provider Screen and/or Patient Screen that is located in the interior of the medical kiosk. If a video image option is available for display on the Provider Screen and/or Patient Screen that is located in the interior of the medical kiosk, the medical provider via the Provider Application can turn off such video image on the Provider Screen and/or Patient Screen; however, this is not required. The medical provider via the Provider Application can capture images and/or data generated by the thermometer and/or video camera in the medial kiosk for placement in the patient's file; however, this is not required. The medical provider via the Provider Application can optionally annotate an image once captured (e.g., use the paint brush and font icons to modify image, etc.) and/or show the captured image to the patient; however, this is not required. The medical provider and/or patient can request assistance by the medical assistant, when available, if so required. In one non-limiting arrangement, the temperature must register between 95.0 degrees and 105.9 degrees; however, this is not required. Temperatures under or above 95.0 degrees or some other temperature and 105.9 degrees or some other temperature can be designed to create an error and request the patient to manually enter their temperature, request the patient to try again to take a proper temperature, and/or request the medical assistant to assist the patient related to the thermometer; however, this is not required. The dermascope, when used, is used to visualize body surface, skin, hair, scalp, eyes, and/or throat with magnification and/or illumination. The image generated by the dermascope in the medical kiosk can be displayed on the Patient's Screen and/or Provider's Screen in the medical kiosk and/or on the screen being used by the medical provider; however, this is not required. The transmission of the signal can be by various means (e.g., hardwire, USB, Bluetooth, Wi-Fi, etc.). On the interior of the medical kiosk, the dermascope can be contained in the first medical device cabinet on the right in the medical kiosk, when a cabinet is used; however, this is not required. The dermascope can be activated by the medical provider via the Provider Application by the medical provider selecting the dermascope icon on the provider's screen; however, the dermascope can be activated by other or additional arrangements or always be on. In one non-limiting arrangement, when the medical provider via the Provider Application selects the dermascope icon on the provider's screen, the cabinet door in the medical kiosk for the dermascope opens (e.g., drops down, etc.) to enable the patient in the medical kiosk to access the dermascope; however, this is not required. The medical provider can provide instructions to the patient on how to use the dermascope (e.g., Once the patient has pulled out the device, request for patient to place the dermascope device at the appropriate place, then request for the patient to adjust the focus dial. The focus dial is on the top handle of the dermascope. The patient can push it to the top and then dial it down to determine appropriate focus, etc.) and/or instructions for use of the dermascope can be provided on the Patient Screen and/or Provider Screen in the medical kiosk; however, this is not required. When the dermascope is selected, a video image can be designed to appear on the Provider Application (e.g., medical provider's screen) and/or the Provider Screen and/or Patient Screen that is located in the interior of the medical kiosk. If a video image option is available for display on the Provider Screen and/or Patient Screen that is located in the interior of the medical kiosk, the medical provider via the Provider Application can turn off such video image on the Provider Screen and/or Patient Screen; however, this is not required. The medical provider via the Provider Application can capture images and/or data generated by the dermascope and/or video camera in the medial kiosk for placement in the patient's file; however, this is not required. The medical provider via the Provider Application can optionally annotate an image once captured (e.g., use the paint brush and font icons to modify image, etc.) and/or show the captured image to the patient; however, this is not required. The medical provider via the Provider Application can adjust the focus of the dermascope; however, this is not required. As can be appreciated, the patient can adjust the focus of the dermascope; however, this is not required. The medical provider and/or patient can request assistance by the medical assistant, when available, related to the dermascope if so required. The pulse oximeter, when used, is used for measuring the amount of saturated hemoglobin the tissue capillaries by transmitting a beam of light through the tissue to a receiver. The image generated by the pulse oximeter in the medical kiosk can be displayed on the Patient's Screen and/or Provider's Screen in the medical kiosk and/or on the screen being used by the medical provider; however, this is not required. The transmission of the signal can be by various means (e.g., hardwire, USB, Bluetooth, Wi-Fi, etc.). On the interior of the medical kiosk, the pulse oximeter can be contained in the second medical device cabinet on the right in the medical kiosk, when a medical cabinet is used; however, this is not required. The pulse oximeter can be activated by the medical provider via the Provider Application by the medical provider selecting the pulse oximeter icon on the provider's screen; however, the pulse oximeter can be activated by other or additional arrangements or always be on. In one non-limiting arrangement, when the medical provider via the Provider Application selects the pulse oximeter icon on the provider's screen, the cabinet door in the medical kiosk for the pulse oximeter opens (e.g., drops down, etc.) to enable the patient in the medical kiosk to access the pulse oximeter; however, this is not required. The medical provider can provide instructions to the patient on how to use the pulse oximeter (e.g., Provider: Once the patient has pulled out the device, request for patient to place the device on their index finger as illustrated by the diagram on the top on the device. The picture is a fingernail print which should align to the patient's fingernail, etc.) and/or instructions for use of the pulse oximeter can be provided on the Patient Screen and/or Provider Screen in the medical kiosk; however, this is not required. When the pulse oximeter is selected, a video image can be designed to appear on the Provider Application (e.g., medical provider's screen) and/or the Provider Screen and/or Patient Screen that is located in the interior of the medical kiosk. If a video image option is available for display on the Provider Screen and/or Patient Screen that is located in the interior of the medical kiosk, the medical provider via the Provider Application can turn off such video image on the Provider Screen and/or Patient Screen; however, this is not required. The medical provider via the Provider Application can capture images and/or data generated by the pulse oximeter and/or video camera in the medial kiosk for placement in the patient's file; however, this is not required. The medical provider via the Provider Application can optionally annotate an image once captured (e.g., use the paint brush and font icons to modify image, etc.) and/or show the captured image to the patient; however, this is not required. The medical provider via the Provider Application can adjust the focus of the pulse oximeter; however, this is not required. As can be appreciated, the patient can adjust the focus of the pulse oximeter; however, this is not required. The medical provider and/or patient can request assistance by the medical assistant, when available, related to the pulse oximeter if so required. The scale, when used, is used to obtain the weight of the patient. The weight that is transmitted by the scale can be displayed on the Patient's Screen and/or Provider's Screen in the medical kiosk and/or on the screen being used by the medical provider; however, this is not required. The transmission of the signal from the scale can be by various means (e.g., hardwire, USB, Bluetooth, Wi-Fi, etc.). As can be appreciated, the weight can be manually entered. On the interior of the medical kiosk, when the kiosk has an interior enclosure, the scale can be located on the floor of the medical kiosk; however, this is not required. The patient's weight can be collected during the vitals capture process which is prior to the consultation with the medical provider; however, this is not required. If the medical provider wants the patient's weight to be retaken or if the patient's weight has not already been taken, the scale can be activated or reactivated by the medical provider via the Provider Application by the medical provider selecting the thermometer icon on the provider's screen; however, the scale can be activated by other or additional arrangements. In one non-limiting arrangement, when the medical provider via the Provider Application selects the scale icon on the provider's screen, information from the scale or information that is manually entered is conveyed to the medical provider; however, this is not required. The medical provider can provide instructions to the patient on how to use the scale (e.g., Request for the patient to step on the scale and wait for the recording to be completed, etc.) and/or instructions for use of the scale can be provided on the Patient Screen and/or Provider Screen in the medical kiosk; however, this is not required. When the scale is selected, a video image can be designed to appear on the Provider Application (e.g., medical provider's screen) and/or the Provider Screen and/or Patient Screen that is located in the interior of the medical kiosk. If a video image option is available for display on the Provider Screen and/or Patient Screen that is located in the interior of the medical kiosk, the medical provider via the Provider Application can turn off such video image on the Provider Screen and/or Patient Screen; however, this is not required. The medical provider via the Provider Application can capture images and/or data generated by the scale and/or video camera in the medial kiosk for placement in the patient's file; however, this is not required. The medical provider via the Provider Application can optionally annotate an image once captured (e.g., use the paint brush and font icons to modify image, etc.) and/or show the captured image to the patient; however, this is not required. The medical provider and/or patient can request assistance by the medical assistant, when available if so required. As can be appreciated, a patient can weigh himself/herself on the scale and then manually enter the data for use by the medical provider; however, this is not required. In one non-limiting arrangement, the weight must register no greater than 501 pound or some other upper limit; however, this is not required. Weights that are above the upper weight limit can be designed to create an error and request the patient to manually enter their weight, request the patient to try again to take a proper weight, and/or request the medical assistant to assist the patient related to the scale; however, this is not required. The scale can be positioned in any location in the medical kiosk (e.g., floor, bench, chair, etc.). A lower weight limit can also be used to create an error which requires the user to try again and obtain a proper weight. In one non-limiting embodiment, the scale can be integrated into the medical kiosk in such a way that it at least partially incorporated in on the floor of the medical kiosk. In one non-limiting design, the scale is positioned flush with the floor so that it poses no safety challenges for the user/patient in the medical kiosk; however, this is not required. As can be appreciated, the size, shape, and type of scale are non-limiting. The scale in the floor of the kiosk can also optionally be used as designated standing location of a patient so that a height of the patient can be obtained by use of one or more cameras and/or other electronic devices in the medical kiosk; however, this is not required. The blood pressure cuff, when used, is used to determine the arterial pressure of the systemic circulation. The blood pressure cuff can also be used to obtain the pulse rate of the patient. The information that is transmitted by the blood pressure cuff can be displayed on the Patient's Screen and/or Provider's Screen in the medical kiosk and/or on the screen being used by the medical provider; however, this is not required. The transmission of the signal from the scale can be by various means (e.g., hardwire, USB, Bluetooth, Wi-Fi, etc.). On the interior of the medical kiosk, the blood pressure cuff can be contained in the third medical device cabinet on the right in the medical kiosk, when a medical cabinet is used; however, this is not required. Alternatively, the blood pressure cuff can be kept with the medical attendant, when available, and the medical attendant then assists the patient in placing the blood pressure cuff on the patient prior to the patient entering the medical kiosk or while the patient is located in the medical kiosk; however, this is not required. The patient's blood pressure can be collected during the vitals capture process which is prior to the consultation with the medical provider; however, this is not required. If the medical provider wants the patient's blood pressure to be retaken or if the patient's blood pressure has not already been taken, the blood pressure cuff can be activated or reactivated by the medical provider via the Provider Application by the medical provider selecting the blood pressure cuff icon on the provider's screen; however, the blood pressure cuff can be activated by other or additional arrangements. In one non-limiting arrangement, when the medical provider via the Provider Application selects the blood pressure cuff icon on the provider's screen, the cabinet door in the medical kiosk for the blood pressure cuff opens (e.g., drops down, etc.) to enable the patient in the medical kiosk to access the blood pressure cuff if the blood process cuff is located in a medical device cabinet; however, this is not required. Alternatively, the selection of the blood pressure cuff results in information from the blood pressure cuff being sent to the medical provider. As can be appreciated, if the blood pressure cuff is not located in a medical cabinet, that patient can obtain the blood pressure cuff from the location in which blood pressure cuff currently exists (e.g., in the medical kiosk, next to the medical kiosk, etc.) or the medical assistant, when available, can provide the blood pressure cuff to the patient if the medical attendant is retaining the blood pressure cuff. In another non-limiting arrangement, the medical kiosk can optionally include an ambidextrous blood pressure cuff. The cuff arm can optionally be mounted to the desk top to enable the blood pressure cuff to be moved from side to side of the desk top; however, this is not required. Such movement of the blood pressure cuff enables the blood pressure cuff to be positioned so that the left arm or right arm of a user can be inserted into the blood pressure cuff when the user is facing the desk top. As can be appreciated, when a blood pressure cuff is included in the medical kiosk, it can be mounted and/or positioned in the medical kiosk in a variety of ways. Information to and from the blood pressure cuff, when used, can be transmitted by wire and/or wirelessly to one or more computers, processors, storage devices, etc. in the medical kiosk and/or to a location remote from the medical kiosk. The ambidextrous blood pressure cuff, when used, may be made of high-strength plastic or any other sufficiently rigid and/or strong material (e.g., metal, composite material, etc.). The cuff arm can be designed to be manually moved by the patient/user, be remotely moved by the medical provider and/or medical kiosk assistant, and/or be electronically moved by the patient/user. The blood pressure cuff can be designed to be activated/deactivated by the patient/user and/or remotely by the medical provider and/or medical kiosk assistant, or always be on. The medical provider can provide instructions to the patient on how to use the blood pressure cuff (e.g., The blood pressure cuff is most effective when the patient fully places their bicep in the cuff, with the palm of their hand facing the ceiling, etc.) and/or instructions for use of the blood pressure cuff can be provided on the Patient Screen and/or Provider Screen in the medical kiosk; however, this is not required. When the blood pressure cuff is selected, a video image can be designed to appear on the Provider Application (e.g., medical provider's screen) and/or the Provider Screen and/or Patient Screen that is located in the interior of the medical kiosk. If a video image option is available for display on the Provider Screen and/or Patient Screen that is located in the interior of the medical kiosk, the medical provider via the Provider Application can turn off such video image on the Provider Screen and/or Patient Screen; however, this is not required. The medical provider via the Provider Application can capture images and/or data generated by the blood pressure cuff and/or video camera in the medial kiosk for placement in the patient's file; however, this is not required. The medical provider via the Provider Application can optionally annotate an image once captured (e.g., use the paint brush and font icons to modify image, etc.) and/or show the captured image to the patient; however, this is not required. The medical provider via the Provider Application can start and/or stop the blood pressure operation; however, this is not required. As can be appreciated, the patient can start and/or stop the blood pressure operation; however, this is not required. The medical provider and/or patient can request assistance by the medical assistant related to the blood pressure cuff if so required.

In another and/or alternative non-limiting aspect of the invention, the novel method and apparatus for providing medical services, diagnoses, health, and/or wellness advice to individuals can include a scheduling application that 1) enables a medical provider to input the day and time the medical provider is available to conduct a visit with a patient in a medical kiosk, 2) obtains or collects information on appointments that have been made by one or more patients at one or more medical kiosks, and 3) assigns a medical provider to a particular appointment that has been made at a particular medical kiosk; however, this is not required. The scheduling application can be designed to enable a patient to select a particular medical provider, gender of a medical provider, specialty of a medical provider, a medical provider that can speak a certain language, etc.; however, this is not required. The scheduling application can be designed to allow a medical provider to refuse an appointment with a particular patient; however, this is not required. If the medical provider refuses an appointment, the scheduling application can be designed to attempt to schedule a different appointment for the newly available time slot of the medical provider, or can block off such time and not reschedule a new appointment for such time period; however, this is not required. The scheduling application can be designed to select a medical provider for a particular appointment based on a set algorithm (e.g., available medical provider that has the largest time since last appointment, available medical provider that accepts patient's insurance, available medical provider that is located closest to home residence of patient, etc.) and/or by a random process. The scheduling application can be designed to attempt to select a new medical provider for a particular appointment if the originally selected medical provider is unable and/or unwilling to conduct a visit with a patient in the medical kiosk; however, this is not required.

In still another and/or alternative non-limiting aspect of the invention, the novel method and apparatus for providing medical services, diagnoses, health, and/or wellness advice to individuals can include a medication adherence application; however, this is not required. The medication adherence application can be designed to reduce medication errors. The medication adherence application can include a reminder for patients to take their medications and then optionally the system can automatically log when the patient takes their medication; however, this is not required. The medication adherence application can also include the ability to track a patient's compliance to taking their medication on time and/or provide automatic progress reports; however, this is not required. The medication adherence application can also include the ability to remind the patient to timely take medications (e.g., text reminder, email reminder, phone call reminder, twitter reminder, video conference reminder, etc.); however, this is not required. The medication adherence application can also include the ability to refer the patient to a pharmacist to answer any questions and/or for additional consultation; however, this is not required. Each user/patient utilizing the medical kiosk can be automatically enrolled in the e-script network, which network sends their prescriptions to the pharmacy of their choice; however, this is not required. The medication adherence application can be used to assist in improving patient outcomes and/or satisfaction. The medication adherence application of the present invention can be designed to be used on a screen in the medical kiosk, and/or on a computer screen and/or mobile device; however, this is not required. The medication adherence application of the present invention can be designed to generate a screen that includes one or more main buttons (e.g., 1) Speak to a ______ (e.g., Walgreens, CVS, Wal-Mart, etc.) Pharmacist now, 2) Change my medications alerts, 3) Learn about controlling my ______ (e.g., cholesterol, high blood pressure, diabetes, etc.), 4) Check orders status, 5) Refill Prescriptions, etc., 6) Transfer prescriptions to ______ (e.g., Walgreens, CVS, Wal-Mart, etc.), 7) Recommended dosage, 8) Period for taking medications, 9) Frequency for taking medications, 10) Information about medications, 11) Generic brands available for medications, 12) Request appointment to speak with a medical provider, 13) Entry of compliance information regarding medication usage by patient, etc.); however, this is not required. The screen can include additional buttons (e.g., help button, Finish button, etc.); however, this is not required. The screen can include advertising information; however, this is not required. The selection of one or more of the button can result in additional screens appearing based on the selected button; however, this is not required. If a user/patient needs further guidance from the pharmacist regarding the prescribed medication, the user/patient can select the first category button to request such information; however, this is not required. The patient/user can be provided a number to call and/or allow the user to send an email, make a phone call, etc. regarding the question. The user/patient can be provided the option to set a new appointment to meet with a medical provider regarding the medication; however, this is not required. The user/patient can be provided the option to change the medication alerts; however, this is not required. Such change can be requested by telephone, email, text, etc. The frequency and/or type of alert and/or the manner in which the alert is sent to the user/patient (e.g., email, twitter, phone message, text, etc.) may also be modified by the user/patient; however, this is not required. The user/patient may be provided the option to obtain information about certain medical conditions (e.g., cholesterol, blood pressure, migraines, back pain, arthritis, allergies, flu, etc.); however, this is not required. The user/patient may be provided the option to obtain the order status, shipment status, etc. for a particular medication; however, this is not required. If allowable by current medication guidelines and/or medical plans, the patient may request to fill their prescriptions directly through the application; however, this is not required. The user/patient may be provided the option to change their preferred prescription location; however, this is not required. The user/patient may be provided the option to enter information as to whether the prescribed medications are being timely taken and in the proper amounts; however, this is not required. Periodic reports regarding medication compliance can be generated and provided to the user/patient via phone, email, text, twitter, etc.; however, this is not required. The medication adherence application of the present invention can be used to allow the patient to more easily obtain and understand the correct usage of their prescriptions. In summary, the medication adherence software application allows a user/patient to 1) speak to a pharmacist, 2) change the patient's medications alerts, 3) learn about certain types of medical conditions, 4) check medication orders status, 5) check medication delivery status, 6) refill a prescriptions, 7) transfer prescriptions to a another location, 8) obtain information about recommended medication dosages, 9) obtain information about recommended times to take medications, 10) obtain information about recommended frequency for taking medications, 11) obtain information about medications, 12) obtain information about generic brands available for medications, 13) request an appointment to speak with a medical provider, 14) enter information regarding compliance information regarding medication usage by patient, and/or 15) receive compliance reports for patients regarding medication usage.

In yet another and/or alternative non-limiting aspect of the invention, the novel method and apparatus for providing medical services, diagnoses, health, and/or wellness advice to individuals can include an expandable mounting system for medical devices; however, this is not required. Such an arrangement is generally for kiosks other than personal medical kiosks; however, this is not required. In one non-limiting embodiment of the invention, the medical kiosk includes one or more equipment chambers that can be positioned on or near the front interior wall of the medical kiosk; however, it can be appreciated that one or more equipment chambers can be positioned in other or additional regions of the medical kiosk. The equipment chambers are used to store one or more medical devices (e.g., stethoscope, otoscope, thermometer, dermascope, spirometer, pulse oximeter, heating pad, magnifying glass, tongue depressor, tweezers, blood glucometer etc.). The one or more equipment chambers can also or alternatively be used to include other types of materials (e.g., tissue, Band-Aid, gauze, cotton ball, disinfecting wipe, cortisone cream, antibiotic cream/ointment, alcohol wipe, cotton swab, fabric wrap, etc.). The one or more equipment chambers generally include a door to limit access to the one or more equipment chambers; however, this is not required. The door, when used on one or more of the equipment chambers, can be manually openable/closeable, and/or the doors can be controllably opened/closed remotely by the medical provider and/or medical attendant. Generally, one or more of the doors are controllably opened and/or unlocked by the medical provider during the examination of the user in the medical kiosk; however, this is not required. After the user/patient has left the medical kiosk, the medical attendant, when available or used, can enter the medical kiosk, clean the medical equipment that was handled or used by the prior user/patient, and/or dispose of and/or replace items that were used and/or handled by the prior user/patient; however, this is not required. Thereafter, the medical attendant can restock, replace, and/or reposition the medical equipment and/or non-medical equipment in the equipment chambers and close the equipment chamber doors prior to the next user/patient entering the medical kiosk; however, this is not required. One or more types of medical equipment can be designed to transmit information by wire or wirelessly to electronic components in the medical kiosk and/or to the remotely located medical provider; however, this is not required. In one non-limiting arrangement, the medical kiosk can include six (6) equipment chambers (e.g., medical device cabinets, etc.) having doors, three on each side of the desk top of the medical kiosk. As can be appreciated, a larger or smaller number of equipment chambers can be used and/or the one or more equipment chambers can be positioned in other or additional locations in the medical kiosk. As also can be appreciated, some or all of the equipment chambers can be absent doors. The door on one or more of the equipment chambers can be designed to be unlocked and/or opened remotely by the medical provider and/or medical attendant; however, this is not required. The doors can be designed to automatically lock closed when the doors are closed by the medical attendant and/or medical provider after the user/patient has left the medical kiosk; however, this is not required. Each of the equipment chambers can be designed to include a different piece of medical equipment (e.g., stethoscope, otoscope, thermometer, dermascope, spirometer, and pulse oximeter); however, other or additional equipment and/or materials can be included in one or more of the equipment chambers (e.g., wipes, blood sugar analyzer, tissue, band aide, gauze, urine sample container, medication, defibrillator, blood pressure cuff, an audiometer to test hearing, a 3-lead EKG, a glucometer to measure blood glucose levels, etc.). Alternatively, the equipment chambers can be used to store one or more integrated medical devices that can include, but are not limited to, the following integrated devices: a stethoscope to evaluate heart, lung and/or bowel sounds; a thermometer to record temperature; an otoscope to examine the ear drum, external ear canal, nasal passages, mouth and/or throat; pulse oximeter to measure blood oxygen saturation and/or heart rate; a spirometer and transducer to measure the volume of air inspired and/or expired by the lungs; an audiometer to test hearing; a 3-lead EKG to provide a snapshot of the heart rhythm and/or data regarding stress and/or injury to the heart muscle; and/or a glucometer to measure blood glucose levels. As can be appreciated, other or additional medical devices can be included in the medical kiosk which are inside or outside of the expandable mounting system such as, but not limited to, a scale, lab-on-a-chip finger stick technology, blood analyzer (e.g., blood sugar analyzer, blood toxicity analyzer, blood oxygen content analyzer, etc.), height tape and others; however, this is not required. As can also be appreciated, a larger or smaller number of medical equipment can be used with and/or included in the medical kiosk and/or different types of medical equipment can be used with and/or included in the medical kiosk. The one or more equipment chambers can also or alternatively be used to include other types of material (e.g., tissue, gauze, disinfecting wipe, cotton ball, tongue depressor, tweezers, cortisone cream, urine sample container, etc.); however, this is not required. The one or more equipment chambers generally include a door to limit access to the one or more equipment chambers; however, this is not required. The door, when used, can be manually openable/closeable, and/or the doors can be controllably open/closed remotely by the medical provider or medical attendant; however, this is not required. The expandable mounting system for medical devices can be made of high-strength plastic or any other sufficiently rigid and strong material such as metal, wood, and the like. The configuration and/or size of each the equipment chambers can be the same or different. The one or more doors on the expandable mounting system can be manually opened and/or closed and/or remotely opened and/or closed by a medical provider and/or medical attendant. The one or more medical devices in the one or more chambers or bays can be activated/deactivated remotely by software and/or manually activated/deactivated. The one or more medical devices in the one or more chambers or bays can be connected to a connector in the chambers or bays so as to supply power to the medical device and/or to electronically transmit information between the medical device and a computer, network, storage device, etc. The expandable mounting system can include one or more visual indicators (e.g., light, etc.) to indicate to a user/patient which chamber is to be used and/or is active. A medical attendant and/or medical provider can be used to assist the patient in the operation of one or more medical devices; however, this is not required.

In still yet another and/or alternative non-limiting aspect of the invention, the novel method and apparatus for providing medical services, diagnoses, health, and/or wellness advice to individuals can include digital signage on the exterior of the medical kiosk; however, this is not required. Generally, the personal medical kiosk does not include digital signage; however, this is not required. The medical kiosk can include advertisements that are displayed on the display; however, this is not required. The digital signage, when used, can be displayed through one or more monitors or display screens on the exterior and/or interior of the medical kiosk to able a user/patient/potential user to see one or more of the monitors or display screens. One non-limiting aspect of this invention is that the one or more monitors or display screens can be used to display advertisements that may be arranged by the originating site; however, this is not required. Another non-limiting aspect of this invention is that one or more of the monitors or display screens can be used to display the current wait time for the medical kiosk and/or the list of scheduled appointments; however, this is not required. The digital signage application can allow various companies to advertise via the medical kiosk, including the business where it is located; however, this is not required. The digital signage application can be tailored to display information for specific patients based upon their conditions; however, this is not required. The digital signage application can include the ability to display a scrolling message at the bottom of the screen, based upon the business's preference; however, this is not required. The one or more display screens and/or monitors can be used to provide various types of information (e.g., registration information, information input by the user, advertising information, information about the medical kiosk, information about wait time for a medical kiosk, information as to the order of users waiting to use the medical kiosk, information about whether a medical kiosk is available or in use, cable TV, satellite TV, local broadcast TV, infomercial, medical programs, DVD materials, Blu-ray materials, etc.). Patients waiting to enter the medical kiosk can be allowed to view the various advertisements and/or other types of information displayed on the one or more screens; however, this is not required. The patients can see the wait time for their appointment and/or their position in the appointment queue when such information is displayed on the one or more video screens; however, this is not required. Any message necessary for the patient to see can also be displayed upon the screen in the interior of the medical kiosk; however, this is not required (e.g., “Welcome (Patient Name)”, “Your Medical Provider (Provider Name) Will Begin Your Visit Once You Have First Captured Some Basic Vitals”, etc.). The one or more interior screens are primarily utilized for video conferencing between the patient and the provider, patient data input, and patient instructions, but can also be used to display advertisements and/or other information during periods where the medical kiosk has no appointment queued; however, this is not required. The digital signage application can allow businesses to display advertisements and/or current promotions in a way that will attract the consumer's attention; however, this is not required. The ability to tailor the signage to individual patients and/or population groups makes it very adaptable; however, this is not required. The information on the display can be in any language. The information on the display can include subtitles, etc.; however, this is not required.

In another and/or alternative non-limiting aspect of the invention, the novel method and apparatus for providing medical services, diagnoses, health, and/or wellness advice to individuals can include an exterior check-in registration station via an integrated touch screen monitor; however, this is not required. The personal medical kiosk generally does not include the use of an exterior check-in registration station. The novel method of the present invention can include the use of a medical kiosk to enable the patient to conveniently communicate with the medical provider. In one non-limiting embodiment, the medical kiosk has an exterior check-in registration station. The check-in registration station can include a key pad and/or key board for identification and/or data entry, a touch screen for identification and/or data entry, microphone and/or voice recognition software for identification and/or data entry, fingerprint scanner for identification and/or data entry, and/or retina scanner for identification and/or entry; however, this is not required. As can be appreciated, other or additional devices can be included on the medical kiosk for identification and/or data entry. The medical kiosk having an exterior check-in registration station can be used by the patient to enter/convey basic information about the patient. Such information includes, but is not limited to, a) patient name, b) patient address, c) patient contact information, d) patient age, e) patient sex, f) patient height, g) patient weight, h) patient medical history, i) current medicines used by patient, j) reason(s) for visit by patient, k) patient current symptoms, l) patient insurance information, m) patient payment information, n) consent forms, and/or o) patient's current doctor. As can be appreciated, other or additional information can be input/conveyed by the patient. The medial kiosk can be designed to provide information to the patient prior to and/or during the inputting/conveying of information by the patient to the medical kiosk. In one non-limiting embodiment of the invention, the medical kiosk can include audio and/or visual instructions and/or displays used to provide a) information about the medical kiosk, b) how to use the medical kiosk, c) how to properly input/convey information to the medical kiosk, d) provide instructions and/or interactions with the patient during the inputting/conveying of information by the patient to the medical kiosk, e) the wait time for the patient's use of the medical kiosk, f) a list of patient's waiting to use the medical kiosk, g) the types of conditions that can be treated by the medical kiosk, h) the type of medical devices that can be used with the medical kiosk, i) how to use medical devices with the medical kiosk, j) information about who is qualified to use the medical kiosk, and/or k) information regarding whether the medical kiosk is in use or is available. In another and/or alternative non-limiting embodiment of the invention, the medical kiosk can include light and/or sound indicators to provide information regarding 1) whether the medical kiosk is in use or is available, 2) whether information is required to be entered and/or is improperly entered and/or correctly entered in the medical kiosk, and/or 3) whether a medical device is properly or improperly connected to the medical kiosk; however, this is not required. In still another and/or alternative non-limiting embodiment of the invention, the medical kiosk can include a notification system to a patient that the medical kiosk is available or will soon be available; however, this is not required. Such notification can be sent via email, phone, pager, internet, etc. Such notification system can be useful when the medical kiosk is not currently available to the patient. The patient can input the information into the medical kiosk and then go home, run other errands, etc., and then be later notified when the medical kiosk is available or will soon be available. The medical kiosk and/or notification system can also be used to inform the patient when and/or where other medical kiosks are available; however, this is not required. This service, when available, can be used to inform the patient that a nearby medical kiosk has a shorter wait period or is currently available, thus providing the patient with the option of traveling to another available medical kiosk instead of waiting for the current medical kiosk to become available; however, this is not required. This service, when available, can also be used to inform the patient when a prescription is ready for pickup and/or for conveying prescription information to the patient; however, this is not required. This service, when available, can also be used to inform the patient when a follow-up visit is due and/or scheduled; however, this is not required. As can be appreciated, the notification system can be used to provide information to patients about medication, medication compliance, when medication should be taken, or for other or additional services. The medical kiosk can include an exterior registration station for check-in via a touch-screen monitor (e.g., monitor, etc.). The exterior registration station is typically not used with a personal medical kiosk. The monitor or touch-screen monitor of the exterior registration station can be mounted at a height and angle that meets ADA compliance and is easily accessible to individuals in wheel chairs; however, this is not required. The touch screen monitor can include a built-in privacy filter for HIPAA compliance; however, this is not required. In one non-limiting embodiment, the front panel of the medical kiosk includes a registration station. The registration station can include a touch screen, a display screen, and an optional frame that such components can be mounted thereto; however, this is not required. The shape of the frame, when used, is non-limiting. The frame, when used, can be designed to be easily removed from the front panel to enable servicing, repair, replacement, etc. of one or more components of the registration station; however, this is not required. As can be appreciated, the registration station can also or alternatively include other or optional features (e.g., additional display screen, additional touch screen, lights, buttons, switches, camera, speakers, microphone, keyboard, scanner, receiver, transmitter, credit card/debit card or other some other card reader, smart phone or other smart device reader/scanner, finger and/or eye scanner, shelf, printer, storage cavity, service access door, motion sensor, sound sensor, temperature sensor, logos, etc.); however, this is not required. The touch screen, when used, is generally used to allow a user to enter in information about the user (e.g., age, sex, contact information, payment information, medical history, medical issue, etc.); however, this is not required. The touch screen can be substituted for a keyboard; however, this not required. The frame can be designed to mount the touch screen at some angle (e.g., 10-80) relative to the front plane of the front panel; however, this is not required. The frame optionally includes one or more side sections that can include one or more other or optional features of the registration station; however, this is not required. As can be appreciated, one or more other or optional features of the registration station can also or alternatively be located on other regions of the registration station. The touch screen can display various types of information (e.g., electronic keyboard, instructions on how to register, questions that are displayed during registration, instructions during registration, information displayed to user during registration, various templates, various menus, various lists of information, etc.); however, this is not required. As can be appreciated, the medical kiosk can be designed to accept voice commands during the registration process; however, this is not required. The display screen can be used to provide various types of information (e.g., registration information, information input by the user, advertising information, information about the medical kiosk, information about wait time for a medical kiosk, information as to the order of users waiting to use the medical kiosk, information about whether a medical kiosk is available or in use, cable TV, satellite TV, local broadcast TV, infomercial, medical programs, DVD materials, Blu-ray materials, etc.). Another display screen can be positioned above the screen used at the registration station and/or at some other location on the exterior of the medical kiosk; however, this is not required. This other display screen can be used to display various types of information (e.g., advertising information, information about the medical kiosk, information about wait time for a medical kiosk, information as to the order of users waiting to use the medical kiosk, information about whether a medical kiosk is available or in use, cable TV, satellite TV, local broadcast TV, infomercial, medical programs, DVD materials, Blu-ray materials, etc.); however, this is not required. In one non-limiting arrangement, the screen used at the registration station and the one or more other displays can be designed to display different types of information (e.g., screen used at the registration station displays information related to the registration process and the one or more other displays are used to display one or more types of information that are different from the information displayed on the screen used at the registration station, etc.); however, this is not required. The size of the screen used at the registration station and the one or more other displays can be the same or different. In one non-limiting arrangement, a user/patient can be allowed to enter payment information at the registration station (e.g., swipes a credit or debit card, etc.); however, it can be appreciated that payment information can also or alternatively be entered inside the medical kiosk, at the optional attendant station, wirelessly or over a network via a smart phone or other device or by a computer connected to a network, etc. If a medical attendant is available, the medical attendant can assist a user during the registration process; however, this is not required. Generally, the medical kiosk includes a single registration station; however, this is not required. As can be appreciated, the registration station can alternatively be located inside the medical kiosk, at the attendant station, on other panels or sidewalls of the medical kiosk, or located remotely from the medical kiosk (e.g., central registration center for use with multiple medical kiosks, etc.). In one non-limiting arrangement, the exterior check-in registration station can be designed to be easily removed from the front panel of the medical kiosk and/or be easily accessed from the interior of the medical kiosk to enable servicing, repair, replacement, etc. of one or more components of the registration station; however, this is not required. The check-in system in accordance with the present invention offers a new way for organizations to meet rising consumer expectations for convenience and, at the same time, improve accuracy and usability of information systems; however, this is not required. The check-in system in accordance with the present invention can result in shorter waiting times for check-in, as well as efficiency gains from increased throughput and fewer errors in keeping patient demographic data up to date; however, this is not required. The check-in system in accordance with the present invention can result in the reduction of risk of patient misidentification and clerical errors at data entry; however, this is not required. The check-in system in accordance with the present invention can improve accuracy in language access for those not fluent in English by offering multiple language options during the check-in process; however, this is not required.

In still another and/or alternative non-limiting aspect of the invention, the novel method and apparatus for providing medical services, diagnoses, health, and/or wellness advice to individuals can include a sanitizing system (e.g., UV system, mist system, etc.) that can be automatically activated and/or activated by the medical attendant, when used, and/or medical provider prior to and/or after a patient has used the medical kiosk; however, this is not required. The personal medical kiosk generally does not include a sanitation system; however, this is not required. The medical kiosk can include a cleaning system designed to clean the interior of the medical kiosk and/or kill/neutralize some of or all germs and/or other microorganisms in the medical kiosk (e.g., air in the medical kiosk, one or more surfaces of the medical kiosk, one or more medical devices, etc.); however, this is not required. One non-limiting cleaning system that can be used is an ultraviolet sanitizing system. As can also be appreciated, a mist sanitizer can also or alternatively be used to fully or partially clean/sanitize one or more portions of the medical kiosk. As can be appreciated, other or additional cleaning systems can be used.

In another and/or alternative non-limiting aspect of the invention, the medical kiosk can be made of one or more materials that resist growth of bacteria, viruses and/or other microorganisms; however, this is not required. In one non-limiting embodiment, the floor, walls and ceiling of the medical kiosk include or are fully made of materials that resist or prevent the growth of bacteria, viruses and/or other microorganisms; however, this is not required. In one non-limiting arrangement, the medical kiosk includes an ultraviolet light sanitization system which can be used in the medical kiosk and which can be activated through software from a remote location and/or by a kiosk attendant; however, this is not required. The ultraviolet light sanitization system can be activated twice daily to ensure the sanitary environment is maintained; however, it can be appreciated, that the ultraviolet light sanitization system can be activated a greater or lesser number of times per day. The location of the ultraviolet light sanitization system, when used, is non-limiting. In one non-limiting arrangement, the ultraviolet light sanitization system can be located in the roof or ceiling of the medical kiosk; however, this is not required. For example, the ultraviolet light sanitization system can be connected or positioned adjacent to the ceiling panel and rear panels; however, this is not required. The ultraviolet light sanitization system can be located in the rear of the medical kiosk where it will be out of the way of the patients utilizing the medical kiosk; however, this is not required. The ultraviolet light sanitizing system generally includes one or more ultraviolet lights that are designed to kill some or all of the germs and/or other microorganisms in the medical kiosk (e.g., air in the medical kiosk, one or more surfaces of the medical kiosk, one or more medical devices, etc.). The ultraviolet sanitizing system can optionally include one or more vents that allow air to flow into and/or out of the ultraviolet sanitizing system to facilitate in the cooling of the ultraviolet sanitizing system; however, this is not required. The sanitization system can be built with high-strength plastics and/or any other sufficiently rigid and strong material such as metal and constructed in a way preventing it from being tampered with by patients or anyone other than the attendant; however, this is not required. Such an arrangement, when used, can facilitate in ensuring that it is not activated incorrectly or at an inopportune time; however, this is not required. The shape, size and/or configuration of the built in sanitization system are non-limiting. The one or more medical attendants, when used, can also or alternatively clean and/or sanitize various regions of the medical kiosk prior to and/or after being used by a patient and/or set up the medical kiosk for a new user; however, this is not required. For example, prior to and/or after one or more patients have entered the medical kiosk, the one or more attendants can clean/sanitize one or more exterior surfaces and/or regions of the medical kiosk (e.g., medical kiosk door, medical kiosk check-in terminal, medical kiosk desk top, medical kiosk exterior walls, medical kiosk touch screen, medical kiosk monitors, seating/tables in waiting area near medical kiosk, etc.); however, this is not required. In an another and/or additional example, prior to and/or after one or more patients have entered the medical kiosk, the one or more medical attendants, when used, can clean/sanitize one or more interior surfaces of the medical kiosk (e.g., medical kiosk door, medical kiosk floor, medical kiosk bench, medical kiosk chair, medical kiosk user terminal, medical kiosk interior desk top, medical kiosk interior walls, medical kiosk touch screen, medical kiosk monitors, medical kiosk instrument doors, medical devices/instruments used by and/or touched by user when in the medical kiosk, any other surface in the interior of the medical kiosk, etc.); however, this is not required. For a personal medical kiosk, the patient can generally clean/sanitize the personal medical kiosk and/or medical devices prior to, during and/or after use of the personal medical kiosk. In still another and/or additional example, prior to and/or after one or more patients have entered the medical kiosk, the one or more medical attendants, when used, can set up the medical kiosk for a user (e.g., clean/sanitize interior surfaces of medical kiosk; clean/sanitize medical devices/instruments used and/or touched by a prior user; reposition medical devices/instruments into device storage areas; replace disposable components on medical devices/instruments; fix, repair and/or replace fans, UV bulbs, UV devices, etc. in the interior and/or exterior of the medical kiosk; refill cleaning and/or sanitizing fluid; etc.); however, this is not required.

In still yet another and/or alternative non-limiting aspect of the invention, the medical kiosk can include a sanitizing system (e.g., UV system, mist system, etc.) that can be automatically activated and/or activated by the medical attendant, when used, prior to and/or after a patient has used the medical kiosk; however, this is not required. As can also be appreciated, a mist sanitizer can also or alternatively be used to fully or partially clean/sanitize one or more portions of the medical kiosk and/or medical devices. Generally, the germs and/or other microorganisms in the medical kiosk are treated when the interior of the medical kiosk does not include a user. The sanitizing system can optionally include one or more standard lights that can be used to provide illumination in the medical kiosk; however, this is not required. The sanitizing system can optionally include a cooling fan for the one or more components in the sanitation system; however, this is not required. The sanitizing system can optionally include both a UV and a mist sanitizing system; however, this is not required. The sanitizing system can house one or more cameras, speakers, sensors (e.g., temperature sensor, motion sensor, sound sensor, etc.), etc. for use in the medical kiosk; however, this is not required. The sanitizing system includes a shroud that includes vent/light/mist openings to house the components of the sanitizing system; however, this is not required. The shape, size and configuration of the shroud, when used, are non-limiting. When a mist sanitizing system is additionally or alternatively used, one or more mist nozzles can be located in one or more regions of the medical kiosk so as to direct the sanitizing mist to desired locations in the medical kiosk (e.g., onto certain surfaces of the medical kiosk, onto certain medical devices, etc.); however, this is not required. The doors to the medical kiosk can be closed and/or locked to prevent a user/patent from entering the medical kiosk during a sanitizing process; however, this is not required.

In yet another and/or alternative non-limiting aspect of the invention, the novel method and apparatus for providing medical services, diagnoses, health, and/or wellness advice to individuals can include a medical kiosk that enables a service operator to easily access the various components of the medical kiosk for purposes of service, repair, maintenance, upgrade, replacement, etc.; however, this is not required In order to offer the comprehensive services the medical kiosk is capable of, there are multiple components that must be functioning at all times; however, this is not required. The various devices that may need service include, but are not limited to, the display screens, the network connection, various other electronics and wiring, fans, lights, electronic switches, backup power supplies, computers, electronic storage devices, doors on the medical equipment compartments, etc. The ability to easily and conveniently access and/or perform maintenance on these components is advantageous to providing the medical kiosk services via the medical kiosk; however, this is not required. To more easily facilitate the service of these components, the medical kiosk can be designed with a moveable front panel; however, this is not required. The front panel can be built on casters, wheel, rail system, etc. to allow the front panel to be easily moved; however this is not required. Such an arrangement is generally not used with a personal medical kiosk; however, this is not required. The front panel can be equipped with a tamper-proof lock to prevent unauthorized personnel from accessing the components located behind the front panel; however, this is not required. A portion of or the entire front panel can be designed to move in order to ensure that when there is a problem with one or more components used in the medical kiosk, the repair of such components does not require the disassembly of the entire or a substantial portion of the medial kiosk to repair the components; however, this is not required. The components of the medical kiosk that are generally difficult or inaccessible when the medical kiosk is fully assemble are 1) the components that form the exterior check-in station, 2) the interior displays or monitors, 3) the interior speakers, the interior cameras, 4) the interior sound jack, 5) the doors on the medical compartments, 6) the interior microphone, 7) the electronics located behind the exterior check-in station, 8) the electronics located behind the interior AV system, and/or 9) the computer and other electronics used for network communication, control and/or storage; however, this is not required. As can be appreciated, there may be other or additional components of the medical kiosk that are generally difficult or inaccessible when the medical kiosk is fully assembled. Access to one or more of these components can be more easily accessed by the movement of the interior front panel of the medical kiosk; however, this is not required.

In still yet another and/or alternative non-limiting aspect of the invention, the novel method and apparatus for providing medical services, diagnoses, health, and/or wellness advice to individuals can include a method and a medical kiosk that enables the medical provider to generate electronic prescriptions and/or for the patient to conveniently select and/or order prescription drugs; however, this is not required. In one non-limiting embodiment, this novel method and apparatus will allow patients to 1) choose between name brand and generic drugs, 2) choose the supply quantity for the prescription (i.e., 30 days supply, 60 days supply, 90 days supply, etc.), 3) choose between picking up the prescription at the pharmacy of their choice or mail delivery of the prescription, 4) enter medical insurance for partial or full payment of the prescription, 5) enter a credit or debit card information to pay for the prescription, 6) enter information for mail delivery of the prescription, 8) enter information to provide automatic reminders to patient regarding refills and/or follow-up medical visits, 9) enter information to enable a patient to be notified when a prescription has been mailed and/or is ready to be picked up at the pharmacy; however, this is not required, 10) obtain a print out and/or electronic version of the prescription written by the medical provider, and/or 11) receive information about the issued prescription (e.g., prescribed use, side effects, etc.) in printout and/or electronic form; however, this is not required. Another non-limiting aspect of this method and apparatus will allow the patient to select the pharmacy where he/she would like to pick up the prescription; however, this is not required. In one non-limiting embodiment of the invention, the medical provider can generate an electronic prescription for a patient. The prescription can include both the generic and name brand drugs along with the patient copay amount for each; however, this is not required. The electronic prescription can provide additional information (e.g., potential savings for selecting certain medications, the dosage amount, the medication dosage, quantity or strength, name of medical provider, help information, coupons, advertising information, etc.); however, this is not required. Once the particular medication brand is selected by the patient, another screen can optionally appear; however, this is not required. For example, the second screen can illustrate the brand of medication selected by the patient/user and/or also provide quantity supply options for the medication, etc.; however, this is not required. The second screen can optionally illustrate additional information such as, but not limited to, the copay amount for the selected medication brand and quantity, the monthly, yearly, etc. savings for selecting a particular medication brand and/or quantity, help information, go back option, advertising information, coupons, generic options for medication, etc.; however, this is not required. The second or a third screen can optionally appear that provides an order confirmation along with the associated savings; however, this is not required. The second or third screen can optionally illustrate additional information such as, but not limited to, the monthly, yearly, etc. savings for selecting a particular medication brand and/or quantity, help information, advertising information, etc. As can be appreciated, other or additional screens can be displayed to the user/patient. This aspect of the invention has the advantage of allowing the user/patient to view and/or select brand or generic drugs, thereby allowing the user/patient to decide which drug option is best for them and their budget; however, this is not required. The medical kiosk and method for using the medical kiosk can thus include a point of purchase prescription workflow application allowing a medical provider to generate prescriptions and optionally allowing patients to select and/or order prescription drugs; however, this is not required. The medical kiosk and method for using the medical kiosk can include a point of purchase prescription workflow application that allows a user/patient to choose his/her drug brand and/or quantity; however, this is not required. The medical kiosk and method for using the medical kiosk can include a point of purchase prescription workflow application that allows a user/patient to select the pharmacy from which to pick up his/her prescription; however, this is not required.

In another and/or alternative non-limiting aspect of the invention, the novel method and apparatus for providing medical services, diagnoses, health, and/or wellness advice to individuals can include headphone jacks; however, this is not required. The one or more headphone jacks allow for hearing-impaired patients to utilize headphones in order to better understand the provider's instructions; however, this is not required. The one or more headphone jacks can also be used to enable additional persons in the medical kiosk (e.g., child's parent, etc,) to listen in on the conversation between the medical provider and patient; however, this is not required. The one or more headphone jacks can also be used to provide additional privacy between the medical provider and patient; however, this is not required. The headphones may be available from the kiosk and/or the medical attendant; however, this is not required. The headphone jack can be placed at a height allowing any person to reach it if necessary; however, this is not required. The headphones generally will be sanitary either through disposable covers or through another form of sanitization; however, this is not required. The headphone jack can be a standard jack to enable a patient to use his/her own headphone; however, this is not required. The medical kiosk can include a wireless system (e.g., Bluetooth technology, IR technology, RF technology, etc.) to transmit sound wirelessly to a patient and/or other person located in the medical kiosk; however, this is not required. The kiosk can also be equipped with a wheelchair accessible ramp; however, this is not required. The medical kiosk can be designed to be fully ADA compliant as a result of these novel innovations; however, this is not required. A medical kiosk that includes one or more headphone jacks and/or a wheelchair accessible ramp can permit the medical kiosk to better serve the public at large; however, this is not required.

In still another and/or alternative non-limiting aspect of the invention, the novel method and apparatus for providing medical services, diagnoses, health, and/or wellness advice to individuals can include a medical kiosk that utilizes high definition video and/or high quality sound to create a conference link between a patient located in the medical kiosk and a medical provider that is located remotely to the medical kiosk; however, this is not required. In one non-limiting aspect of the invention, the medical kiosk includes the use of one or more high definition cameras, one or more high definition monitor(s) and/or high quality speaker(s) built into the medical kiosk and/or used with the medical kiosk; however, this is not required. The one or more cameras located on, near, or within the medical kiosk can be on an adjustable sliding bar allowing camera positioning to create ideal eye contact; however, this is not required. The one or more cameras in the medical kiosk can be used to enable a medical provider to obtain information about a user/patient in the medical kiosk (e.g., height, build, sex, skin color, alertness, body cuts, body infections, body rashes, pupil dilation, hygiene, race, physical condition, emotional condition, metal condition, etc.); however, this is not required. In one non-limiting arrangement, one or more monitors and/or display screens can be positioned on the front interior wall of the medical kiosk; however, this is not required. The monitor is generally used to view the one or more medical providers when the user/patient is located in the medical kiosk; however, this is not required. The shape, size, and thickness of the one or more monitors are non-limiting. One or more cameras can be positioned on the front interior wall, be embedded in the one or more monitor/display screens, and/or be located on a sliding bar allowing it to be positioned relative to the user/patient to create better eye contact with the user/patient in the medical kiosk; however, it can be appreciated that the one or more cameras can be located in other locations. The one or more cameras enable pictures of the user/patient in the medical kiosk to be transmitted to a remotely located medical provider; however, this is not required. The remotely located provider typically has a camera at his/her location so that pictures of the medical provider can be transmitted to the one or more medical screens in the medical kiosk; however, this is not required. As can be appreciated, a projector can be used as a substitute of one or more monitors in the medical kiosk; however, this is not required. One or more speakers can be positioned on the front interior wall of the medical kiosk; however, this is not required. As can be appreciated, one or more speakers can be positioned on other or additional locations in the medical kiosk; however, this is not required. The speakers can be used to enable a user/patient in the medical kiosk to listen to what the medical provider is saying to the user/patient; however, this is not required. One or more microphones are generally included in the medical kiosk to allow the user/patient to communicate with the medical provider; however, this is not required.

In yet another and/or alternative non-limiting aspect of the invention, the novel method and apparatus for providing medical services, diagnoses, health, and/or wellness advice to individuals can include a medical kiosk that is a modular kiosk that is able to be partially or fully broken down; however, this is not required. In one non-limiting embodiment of the invention, the modular medical kiosk can be partially or fully broken down so as to fit through a doorway (e.g., standard 36″ by 80″ doorway, etc.); however, this is not required. The modular design of the medical kiosk, when the medical kiosk has a modular design, can enable the medical kiosk to be set up in various configurations to enable the medical kiosk to be used in various types of spaces; however, this is not required. The modular design of the medical kiosk can be designed to not only accommodate multiple configurations of the medical kiosk, but can also be designed to facilitate in enabling the medical kiosk to be moved into an existing facility and then allowing the assembly of the medical kiosk in such facility without having to modifying the entry ways into or out of the facility; however, this is not required. The medical kiosk can be formed of any number of materials (e.g., plastic, foam, metal, wood, etc.). The modular configuration of the medical kiosk can be such that it can be easily assembled and disassembled so that the medical kiosk can be easily brought into a location and easily set up; however, this is not required. The medical kiosk can be designed to include a floor panel; however, this is not required. The floor panel, when used, is generally a one-piece unit; however, the floor panel can be formed of multiple pieces. The floor panel, when used, can be formed of a durable material (e.g., plastic, metal, wood, composite material, man-made materials, etc.). The floor panel, when used, can be formed of a slightly compressible material to facilitate in the comfort of walking on the floor panel; however, this is not required. The floor panel can have an oval shape; however, other shapes can be used (e.g., circular, square, rectangular, polygonal, etc.). In one non-limiting design, the maximum length of the floor panel is generally 3-15 feet, typically 4-12 feet, more typically about 6-10 feet, and even more typically about 8-9 feet; however, other lengths can be used. In one non-limiting design, the maximum width of the floor panel is generally 3-10 feet, typically 4-8 feet, and more typically about 4-6 feet; however; other widths can be used. In one non-limiting design, the top surface area of the floor panel is generally 10-150 sq. ft., typically 15-80 sq. ft., and more typically about 50-60 sq. ft.; however, other surface areas of the floor panel can be used. The floor panel can be sized to enable a user in a wheelchair to enter the medical kiosk and turn and/or fully maneuver in the medical kiosk while sitting in the wheelchair; however, this is not required. In one non-limiting design, the thickness of the floor panel is generally about 0.1-5 inches, and typically about 0.25-3 inches; however, other thicknesses of the floor panel can be used. A ramp can be optionally used to facilitate the entry and exiting of the medical kiosk; however, this is not required. The shape and size of the ramp are non-limiting. The ramp can be made of a similar material as the floor panel; however, this is not required. The ramp generally includes a sloped surface to facilitate in transitioning from a floor surface to the top surface of the floor panel; however, this is not required. The medical kiosk can optionally include one or more benches, stools and/or chairs. When a bench is included in the medical kiosk, the bench is generally positioned on the back interior wall of the medical kiosk; however, this is not required. The bench can be used to allow a parent, guardian, spouse, relative, friend, etc. to sit in the medical kiosk while the user is obtaining medical services in the medical kiosk. The bench can be designed to enable one or more persons to sit on the bench. The bench can optionally include a storage space under the seat of the bench that can be used to store supplies, equipment, etc. for the medical kiosk. A liftable seat section can be used to access the storage space; however, this is not required. When the bench includes a storage space, the bench can include a lock to limit access to the storage space; however, this is not required. As can be appreciated, the medical kiosk can include one or more chairs (not shown) to enable one or more users to sit in the medical kiosk while receiving medical services in the medical kiosk; however, this is not required. The bench is generally about 10-25 inches high, and typically about 16-20 inches high; however, other heights can be used. In one non-limiting arrangement, the medical kiosk can include two front panels, two rear panels, one side wall, and one door system; however, this is not required. The front panels, rear panels, side wall, and door system are generally formed of a durable material (e.g., plastic, metal, wood, composite material, man-made materials, etc.). As can be appreciated, the medical kiosk can be designed to only include a single front panel and/or a single rear panel. As can also be appreciated, the medical kiosk can be designed to include more than two front panels and/or more than two rear panels. As can be appreciated, the medical kiosk can be designed to include more than one side wall and/or more than one door system. As can also be appreciated, a side wall can be substituted for another door system; however, this is not required. The general shape and size of the front and rear panels are the same; however, this is not required. The shape of the front and rear panels can be arcuate; however, this is not required. The radius of curvature the front and rear panels can be about 10-100 inches, typically 15-50 inches, and more typically about 20-35 inches; however, other radius of curvatures can be used. The front and rear panels can have an angle of curvature of about 90° or a quarter of a circle; however, it can be appreciated that one or both rear and/or front panels can have different angles of curvature. The general shape and size of the side wall and the door system are generally the same; however, this is not required. The side wall and door system can lie in a generally flat plane; however, this is not required. The front and rear panels and the side wall and door system can form a generally oval shape for the medical kiosk; however, this is not required. The two front panels and two rear panels can have the same or similar footprint; however, this is not required. The side wall and the door system can have the same or similar footprint; however, this is not required. The similarity in the shape and footprint of the wall components of the medical kiosk, when used, enables the medical kiosk to be assembled in a manner that is convenient for the facility that will include the medical kiosk. For example, if the door system needs to be positioned on the left side of the medical kiosk, instead of the right side, the similarly shaped side wall and door system enables the medical kiosk to be assembled in such a manner. Also, if the optional registration station of the medical system needs to be placed on the left side or right side or on the rear end of the medical kiosk instead of the front end, the similarly shaped front and rear panels can be easily exchanged to create such configuration for the medical kiosk. The modular medical kiosk not only accommodates multiple configurations of the medical kiosk, it also can facilitate in enabling the medical kiosk to be moved into an existing facility and then assembling the medical kiosk in such facility without having to modify the entryways into or out of the facility; however, this is not required. The thickness and height of the front panels, rear panels, side wall and door system are non-limiting. In one non-limiting embodiment, the maximum height of the front panels, rear panels, side wall and door system is about 5-12 ft., typically about 6-9 ft., and more typically about 7-8 ft.; however, other heights can be used. In one non-limiting embodiment, the thickness of the front panels, rear panels, side wall and door system is generally about 0.5-10 inches, typically about 1-5 inches, and more typically about 1-2 inches; however, other thicknesses can be used. The front panels, rear panels, side wall and door system can optionally include insulation, sound dampening material, etc.; however, this is not required. The front panels, rear panels, side wall and door system can be designed to be connected together in a variety of ways (e.g., bolted/screwed together, latched together, snap fitted together, press fitted together, etc.). Generally, the arrangement is used to connect together the front panels, rear panels, side wall and door system to enable easy connecting and disconnecting of the front panels, rear panels, side wall and door system from one another. One or more of the front panels, rear panels, side wall and door system can include openings, windows, transparent/semi-transparent regions that allow for ventilation, illumination, and/or viewing; however, this is not required. Generally, front panels, rear panels, side wall and door system are mostly or fully formed of opaque or non-transparent materials so as to ensure the privacy of the user in the medical kiosk; however, this is not required. The configuration of the door system is non-limiting. The door system can include a frame and two doors; however, it can be appreciated that the door system only includes a single door. Each door can include a handle or grasp cavity on one or both sides of the one or both doors. The one or more doors can be designed to open and close in a variety of ways (e.g., swing open and closed, slide open and closed on a top/bottom rail system, etc.). As can be appreciated, the one or more doors for the medical kiosk can also or alternatively be positioned on one or more of the front or rear panels; however, this is not required. In one non-limiting embodiment, the maximum height of the doors is generally about 5-9 ft., and typically about 6-7 ft.; however, other heights can be used. In one non-limiting embodiment, the maximum width of the entry provided by the one or more doors when fully open is generally about 15-60 inches, typically about 25-55 inches, and more typically about 30-50 inches; however, other widths can be used. The door opening is generally selected to enable a standard wheelchair to pass through the opening; however, this is not required. The medical kiosk can optionally include an exterior attendant station that is connected to and/or positioned near the medical kiosk. The exterior attendant station can be used by one or more medical attendants, medical providers, etc. A desk can be connected to and/or positioned next to an exterior wall of the medical kiosk; however, this is not required. The desk, when used, can be formed of one or more pieces. When the desk is designed to be connected to an exterior wall of the medical kiosk, such connection arrangement is not limited (e.g., screw, bolt, clamp, press fit, snap arrangement, etc.). The desk can include a desk top, one or more legs, one or more shelf regions, one or more drawers, etc.; however, this is not required. One or more chairs (not shown) can be used to allow one or more medical attendants, medical providers, etc. to sit at the desk; however, this is not required. The desk, when used, can be positioned at or adjacent to one or both front panels; however, this is not required. The desk can be designed to include one or more drawers, shelves, doors, etc. One or more locks can be included on the desk; however, this is not required. The desk can be used to support a monitor, computer, power supply, power strip, printer, router, network switcher, key board, mouse, printer paper, medical supplies, sanitation supplies, refrigerator, freezer, scanner, credit card/debit card reader, data port, lights, desk supplies, etc. The freezer and/or refrigerator, when used, can contain immunizations, medications, urine samples, blood samples, etc.

In still yet another and/or alternative non-limiting aspect of the invention, the novel method and apparatus for providing medical services, diagnoses, health, and/or wellness advice to individuals can include a medical kiosk that includes a ceiling panel; however, this is not required. The ceiling panel can be formed of one or more pieces. The ceiling panel can be formed of a transparent or semi-transparent material to allow light to enter and illuminate the interior of the medical kiosk; however, this is not required.

In still yet another and/or alternative non-limiting aspect of the invention, the novel method and apparatus for providing medical services, diagnoses, health, and/or wellness advice to individuals can include a medical kiosk that is made of one or more materials that resist or prevent the growth of bacteria, viruses and/or other microorganisms; however, this is not required. In one non-limiting embodiment, the floor, walls and ceiling of the medical kiosk include or are fully made of materials that resist or prevent the growth of bacteria, viruses and/or other microorganisms; however, this is not required.

In still another and/or alternative non-limiting aspect of the invention, the novel method and apparatus for providing medical services, diagnoses, health, and/or wellness advice to individuals can include a medical kiosk that includes a payment center that enables a user to pay for medical services, prescriptions, medical equipment, medical accessories, etc. prior to and/or after the user uses the medical kiosk; however, this is not required. The payment center can be in any form (e.g., credit card reader, mobile phone scanner, transmitter/receiver device, electronic scanner, cash receiver, etc.). The payment center may include a touch pad, keyboard, scanner, receiver, transmitter, credit card/debit card or some other card reader, smart phone or other smart device reader/scanner, finger and/or eye scanner, monitor, chair, table, shelf, printer, instructions on how to use the payment center, etc. The payment center can be located on the exterior and/or interior of the medical kiosk. Generally, the user is required to register and pay for the medical services prior to obtaining medical services from the medical provider; however, this is not required. In one non-limiting arrangement, the medical kiosk includes a registration station on the exterior of the medical kiosk (e.g., exterior wall of the medical kiosk, on a table exterior to the medical kiosk, etc.); however, this is not required. As can be appreciated, a user can be allowed to wirelessly connect to the medical kiosk or to some other computer network so as to wirelessly register and/or enter payment information for use of the medical kiosk; however, this is not required. In such an arrangement, a user could register to use a medical kiosk, enter in payment for use of the medical kiosk, set an appointment time for use of the medical kiosk, select a particular medical kiosk to use at some particular location, etc. at some location near or remote from the medical kiosk via a smart phone or other smart device, a computer, etc.

In yet another and/or alternative non-limiting aspect of the invention, the novel method and apparatus for providing medical services, diagnoses, health, and/or wellness advice to individuals can include a medical kiosk that includes an interior cavity or room that provides privacy to the patient when inputting and/or conveying data to the medical kiosk, and/or communicating with a medical provider via an audio and/or video link; however, this is not required. The size, configuration and/or arrangement of the interior cavity or room are non-limiting. The interior cavity or room can include a) one or more speakers, b) one or more microphones, c) one or more video displays, d) one or more data input device, e) one or more chairs and/or other types of seating areas, f) one or more tables, g) one or more doors, h) one or more shelves, i) one or more compartments used to contain medical supplies, medical instruments, etc., j) one or more light switches, k) one or more power outlets, l) sterilization system, m) one or more headphone jacks, n) one or more lights, o) one or more table tops, p) one or more chair and/or benches, q) one or more doors, r) one or more windows, s) walls, floor and/or ceiling, t) one or more vents, u) one or more power outlets, v) one or more light and/or power switches, w) one or more USB and/or data connection outlets, and/or x) one or more fans. As can be appreciated, the interior cavity or room can include other or additional items. The size and configuration of the interior cavity or room can be designed to enable wheelchair access and maneuvering inside the interior cavity or room; however, this is not required. For example, the interior cavity or room can be designed to enable a standard wheelchair to move 90°, 180° and/or 360° while in the medical kiosk. The size and configuration of the interior cavity or room can be designed to provide sufficient room for the patient so that the patient can easily move within the interior cavity or room and/or the patient does not feel cramped or claustrophobic when in the interior cavity or room; however, this is not required.

In still yet another and/or alternative non-limiting aspect of the invention, the novel method and apparatus for providing medical services, diagnoses, health, and/or wellness advice to individuals can be designed to provide a convenient and low-cost structure (e.g., medical kiosk) that can be placed in many different locations, and which enables patients to conveniently access and obtain medical advice and/or care; however, this is not required. Medical providers that are located locally and/or throughout the world can be used to communicate with the patient accessing the medical kiosk. As such, the medical services can be offered year around and at all times so long as there is a qualified medical provider somewhere in the world that is available and is qualified to provide medical assistance via the medical kiosk. Such an arrangement can be more convenient to the medical provider since the medical provider can work from home or from some other convenient location. The arrangement is also convenient to the patient since the patient can access medical assistance via the medical kiosk at the time and place of choosing. Indeed, in rural areas or smaller communities that do not have a local hospital or local doctor's offices nearby, the installation of a medical kiosk in the local drug store, department store, grocery store, etc., results in more accessible and timely medical care for patients in such communities. The costs associated with providing medical care via the medical kiosk may be less than if the patient seeks medical assistance from a hospital, clinic or doctor's office, thus resulting in the patient potentially saving money. As can be appreciated, other or additional advantages may exist by the method of the present invention.

In another and/or alternative non-limiting aspect of the invention, the novel method and apparatus for providing medical services, diagnoses, health, and/or wellness advice to individuals can include the use of a mobile device (e.g., smart phone, tablet, Ipad, Ipod, PDA, etc.) and/or computer (e.g., desktop computer, laptop computer, ultralight computer, notepads, etc.), home PC, smart TVs, and/or entertainment console [e.g., Xbox systems, PS3, PS4, Apple TV, Amazon Fire TV, etc.], etc., or any other device that can access a phone, satellite and/or the internet to enable a patient to 1) conveniently locate an available medical kiosk, 2) schedule an appointment (e.g., date and/or time), 3) pre-register symptoms and/or reasons for visit, 4) set and/or cancel an appointment, 5) received reminders and/or updates regarding appointments, 6) obtain information about medical kiosk availability, 7) obtain information about certain medical provider availability, 8) obtain information about the available medical provider (e.g., name, specialty, etc.), 9) enable the selection of a certain medical provider and/or medical provider in a certain field of medicine, 10) obtain map information, address information and/or hours of operation information regarding selected medical kiosk, 11) locate closest kiosk and/or kiosk availability for a certain medical provider and/or medical provider in a certain field of medicine, 12) presubmit and/or preclear medical insurance, 13) submit payment information, 14) receive information on payment status, 15) receive information on insurance coverage, 16) receive appointment reminders and/or updates, 17) receive prescription information, 18) submit payment information for medical visit and/or prescription, 19) answer surveys regarding the use of the medical kiosk, 20) receive medication reminders, 21) obtain payment from patient, and/or other types of information as previously mentioned above; however, this is not required. The size, shape, configuration and look of the medical kiosk are non-limiting. In one non-limiting embodiment, this invention will allow patients to schedule appointments using a patient portal through a computer and/or mobile device; however, this is not required. Another non-limiting aspect of the application will allow the patient to select the kiosk location for their appointment. While selecting their appointment time and/or location, the patient can also fill out the symptom survey, select the time of their appointment, and/or select a physician if they choose; however, this is not required. The application can also allow the patient to receive appointment reminders via mobile device, smart device, text, phone, cell-phone, web-page, Facebook, twitter, and/or email; however, this is not required. The application may also allow for patients to cancel and/or change their appointment; however, this is not required.

In still another and/or alternative embodiment of the invention, the novel method and apparatus for providing medical services, diagnoses, health, and/or wellness advice to individuals can include a mobile application for a mobile device (e.g., smart phone, tablet, Ipad, Ipod, PDA, blackberry device, mobile note device, Kindle device, Nook device, etc.) and/or computer (e.g., desktop computer, laptop computer, ultralight computer, notepads, etc.), home PC, smart TVs, and/or entertainment console [e.g., Xbox systems, PS3, PS4, Apple TV, Amazon Fire TV, etc.], etc. or any other device that can access a phone, satellite and/or the internet to a portable device that, when launched, gives the user a series of options that can include, but are not limited to, finding a medical kiosk (e.g., medical kiosk, etc.), set medication reminders, visit the home page of the medical kiosk operator, etc.; however, this is not required. As can be appreciated other and/or additional information can be included on the welcome screen of the mobile application and/or computer-based application. The mobile application and/or computer based application can be designed to enable a user to 1) search or and/or locate medical kiosks in one or more communities, towns, cities, states, countries, etc., 2) determine the distance a patient is from a medical kiosk, 3) obtain the directions to a medical kiosk, 4) view the waiting status and/or availability of a particular medical kiosk, 5) obtain a map to the selected kiosk, 6) obtain information about the hours available for the medical kiosk, 7) obtain information about the actual location picture of the medical kiosk, 8) make an appointment with the medical kiosk, 9) obtain information on the hospitals and/or medical providers associated with the medical kiosk, 10) obtain information on which insurance carriers provide coverage for use of the medical kiosk, 11) obtain information on whether a particular medical provider is part of the medical kiosk network of medical providers, 12) obtain public comments about the medical kiosk, 13) contact a helpdesk that provides information about the medical kiosk, 14) schedule an appointment (e.g., date and/or time), 15) pre-register symptoms and/or reasons for visit, 16) enter information required to create an appointment (e.g., personal information, date and time of appointment, particular medical kiosk, particular medical provider, insurance information, payments, ID verification, insurance card, etc.), 17) set and/or cancel an appointment, 18) receive reminders and/or updates regarding appointments, 19) obtain information about the available medical provider (e.g., name, specialty, etc.), 20) enable the selection of a certain medical provider and/or medical provider in a certain field of medicine, 21) locate closest kiosk and/or kiosk availability for a certain medical provider and/or medical provider in a certain field of medicine, 22) presubmit and/or preclear medical insurance, 23) submit payment information, 24) receive information on payment status, 25) receive information on insurance coverage, 26) receive appointment reminders and/or updates, 27) receive medication reminders, 28) receive visit summaries, 29) obtain/review physician/provider ratings from other users or sources, 30) rate/review physician/provider, and/or any other information as mentioned above; however, this is not required. In one particular non-limiting arrangement, the mobile application and/or computer-based application enables a user to schedule an appointment at a medical kiosk. The creation of the appointment can be designed to enable the user to a) select the day and/or the time of the appointment, b) select a particular medical provider or type of medical provider (e.g., pulmonary doctor, gynecologist, etc.), c) provide a reason for visit and/or provide the user's symptoms, and/or d) set appointment and medication reminders; however, this is not required. As can be appreciated, other and/or additional information can be inputted by the user when making an appointment (e.g., sex, age, weight, height, medical history, use of current medications, symptom, allergies, etc.). The mobile application and/or computer-based application can be designed to create an appointment confirmation screen along with text and/or email reminder options; however, this is not required. The confirmation screen, when provided, can provide information about the medical provider; however, this is not required. The confirmation screen, when provided, can be designed to enable the patient to change/cancel the appointment; however, this is not required. Several non-limiting advantages of the present invention include allowing a patient to decide which kiosk location is most convenient for the patient. The mobile application and/or computer-based application may include an override for “first available” in regards to physician selection; however, this is not required. The patient also may be able to check availability of a preferred medical provider and/or the kiosk location; however, this is not required.

In yet another and/or alternative embodiment of the invention, the novel method and apparatus for providing medical services, diagnoses, health, and/or wellness advice to individuals can include a Provider Application wherein the medical provider can contact another medical provider during a patient visit if a special need or question arises during the patient visit wherein the medical provider believes and/or the patient requests a second opinion and/or an opinion from a specialist; however, this is not required. The Provider Application can be designed to enable two or more medical providers to be simultaneously viewed and/or heard by the patient in the medical kiosk; however, this is not required. The Provider Application can be designed to allow only one medical provider at a time to be viewed and/or heard by the patient in the medical kiosk; however, this is not required. The Provider Application can be designed to allow an interpreter to appear to the medical provider and/or patient (e.g., language translator, sign language translator, etc.); however, this is not required. The Provider Application can be designed to allow a medical provider to contact another party (e.g., patient, guardian, relative, etc.) to obtain information, authorization, etc. about/for a patient using the medical kiosk; however, this is not required.

In still yet another and/or alternative embodiment of the invention, the novel method and apparatus for providing medical services, diagnoses, health, and/or wellness advice to individuals can include an Administrator Application allowing a medical administrator to remotely access one or more components of the medical kiosk; however, this is not required. The Administrator Application can optionally include one or more functions selected from the group consisting of 1) allowing remote access to one or more medical devices in the medical kiosk, 2) allowing remote access to one or more computers in the medical kiosk, 3) allowing remote access to one or more routers in the medical kiosk, 4) allowing remote access to one or more displays on the medical kiosk, 5) allowing remote access to one or more power supplies in the medical kiosk, 6) allowing remote access to one or more servers in the medical kiosk, 7) allowing remote access to one or more hard drives in the medical kiosk, 8) allowing diagnostics to be executed from a remote location on one or more electronic components in the medical kiosk (e.g., computer, router, server, battery backup, hard drive, medical devices, electronic locks, fans, displays, speakers, camera, headphone jack, electronic scale, Bluetooth devices, lights, pumps, scanners, touch pad, ID verification devices, printer, etc.), 9) allowing the rebooting and/or reinitializing from a remote location of one or more electronic components in the medical kiosk, 10) allowing for review of the current and/or past status from a remote location of one or more electronic components in the medical kiosk, 11) allowing hardware and/or software updates to be remotely sent and/or loaded onto one or more electronic components in the medical kiosk, and/or 12) allowing software to be loaded onto and/or removed from one or more electronic components in the medical kiosk.

In another and/or alternative embodiment of the invention, the novel method and apparatus for providing medical services, diagnoses, health, and/or wellness advice to individuals can include a pharmaceutical dispensing system that dispenses medications to the patient; however, this is not required. The pharmaceutical dispensing system can be located on the medical kiosk or be a unit separate from the medical device. The pharmaceutical dispensing system can be designed to allow the patient to obtain the medication with or without the assistance of the medical attendant. The pharmaceutical dispensing system can be designed to only dispense medication that was authorized by the medical provider. In one non-limiting arrangement, the medical provider can use the Provider Application to send instructions to the pharmaceutical dispensing system to dispense certain types and/or amounts of medication to the patient; however, this is not required. Such information can be stored by the medical provider in the patient's record; however, this is not required. The medical provider can send notification to the medical attendant that medication is being dispensed to the patient; however, this is not required. The sent information can include type and/or quantity of medication; however, this is not required. Non-limiting medication and/or other materials that can be dispensed by the pharmaceutical dispensing system include antibiotics, aspirin, hydrocodone, simvastatin, Lisinopril, levothyroxine sodium, amlodipine besylate, omeprazole, Azithromycin, Amoxicillin, metformin, Hydrochlorothiazide, Lipitor, Nexium, Plavix, Advair Diskus, Abilify, Seroquel, Singulair, Crestor, Actos, Epogen, Band-Aid, tissue, alcohol wipe, bandage, cold pack, heating pad, etc. The payment for medication and/or materials from the pharmaceutical dispensing system can be by mobile device, payment at the medical kiosk, payment at attendant's station, and/or payment at the pharmaceutical dispensing system and/or by some other method and/or at some other location.

In still another and/or alternative embodiment of the invention, the novel method and apparatus for providing medical services, diagnoses, health, and/or wellness advice to individuals can include a data port and/or wireless receiver that can be used by a patient to download and/or upload information to a medical device on a patient (e.g., heart monitor, heart pacemaker, implantable cardioverter defibrillators, pulse monitor, activity monitor, temperature monitor, etc.); however, this is not required. Such information can be designed to be viewed by the medical provider, and/or data can be sent to the medical device by the medical provider; however, this is not required.

In yet another and/or alternative embodiment of the invention, the novel method and apparatus for providing medical services, diagnoses, health, and/or wellness advice to individuals can be designed to be transported to various locations to be used in an emergency medical station; however, this is not required. For example, after a natural disaster (e.g., flood, hurricane, tidal wave, earthquake, fire, tornado, etc.), the medical kiosk of the present invention can be transported to the area of the incident and then be used as an emergency medical station. As can be appreciated, the medical kiosk can be continuous transported to various locations (e.g., remote rural locations, camp sites, etc.) to enable individuals in such locations to obtain medical assistance.

In still yet another and/or alternative embodiment of the invention, the novel method and apparatus for providing medical services, diagnoses, health, and/or wellness advice to individuals can be designed to allow patients to rate the medical provider and/or to view the ratings of medical providers that have offered services to patients in the medical kiosk; however, this is not required. Such ratings can be used by patients to select a certain medical provider for a visit, if such option is available.

In another and/or alternative embodiment of the invention, the novel method and apparatus for providing medical services, diagnoses, health, and/or wellness advice to individuals can be designed to allow patients to rate the experience received in the medical kiosk and/or to view the ratings of other patients that have used the medical kiosk; however, this is not required.

In still another and/or alternative embodiment of the invention, the novel method and apparatus for providing medical services, diagnoses, health, and/or wellness advice to individuals can be designed to automatically order supplies for the medical kiosk based on the usage of the medical devices in the medical kiosk; however, this is not required. For example, the number of patients using the medical kiosk can be monitored. If a thermometer was used for every visit, a software program can be used to automatically order the disposable component of the thermometer after a certain number of patients have visited the medical kiosk; however, this is not required. In another and/or additional example, software can be used to monitor the number of times a medical door for a medical device has been opened. Based on such number of door openings, a software program can be used to automatically order the disposable component of the medical device in a certain medical compartment after a certain number of door openings has been detected; however, this is not required.

In still yet another and/or alternative embodiment of the invention, the novel method and apparatus for providing medical services, diagnoses, health, and/or wellness advice to individuals can be designed to generate a fresh scent in the medical kiosk and/or select the background music/sound in the medical kiosk and/or select the lighting level in the medical kiosk and/or select the lighting color in the medical kiosk; however, this is not required. The type of scent and/or method of scent delivery are non-limiting. The type of lighting and/or light colors available are non-limiting. The type of music and/or sound available are non-limiting.

In another and/or alternative embodiment of the invention, the novel method and apparatus for providing medical services, diagnoses, health, and/or wellness advice to individuals can be designed to collect information about patients, patient retention, and/or patient referrals for use in the marketing of the medical kiosk and/or to improve/alter/enhance the services provided by the medical kiosk; however, this is not required.

In still another and/or alternative embodiment of the invention, the novel method and apparatus for providing medical services, diagnoses, health, and/or wellness advice to individuals can be designed to print and/or send coupons, advertisements, marketing literature, and/or medical literature to a patient that has used the medical kiosk and/or has registered with the medical kiosk; however, this is not required. Such coupons, advertisements, marketing literature, and/or medical literature can be general in nature and/or targeted to the particular patient and/or type of diagnoses received by the patient. For example, if a prescription has been written by the medical provider, one or more pharmacies may have a coupon sent to the patient to provide the patient with a discount, etc. if the patient fills the prescription at the pharmacy; however, this is not required.

In yet another and/or alternative embodiment of the invention, the novel method and apparatus for providing medical services, diagnoses, health, and/or wellness advice to individuals can be designed to facilitate in scheduling a patient for a tele-medicine visit into a remotely located healthcare kiosk; however, this is not required. Such process can become quite complicated. There can be multiple kiosks, multiple healthcare providers, various regulatory requirements which may limit services in various locations, and/or patient-specific information or requests which may limit available providers. In one non-limiting embodiment of the invention, there is provided a system, method and apparatus that can streamline, speed up, and/or reduce wasted time for scheduling tele-medicine patient-provider visits in multitude of remotely located (or home/mobile based) medical kiosks by automating aspects that assist medical providers in managing waiting queues of patients. The system, method and apparatus of the present invention can also and/or alternatively assist patients with information about expected wait times. The system, method and apparatus of the present invention can also and/or alternatively use wait queuing and/or reservation scheduling for a medical kiosk. The system, method and apparatus of the present invention can also and/or alternatively sort patients by insurance provider and/or type of insurance. This information can be used to identify one or more medical providers that accept the insurance of the user. The information can also or alternatively be used to inform the patient that the user's insurance is not acceptable. The information can also or alternatively be used to inform the patient of the amount of payment of the visit based on various factors (e.g., type of insurance, insurance copay amounts, whether insurance is acceptable, type of medical provider requested, a request for a particular medical provider, time of appointment, day of appointment, type of medical condition, etc.). In accordance with one non-limiting aspect of the present invention, when a patient checks in to a medical kiosk, the patient is automatically placed into a wait queue. Once in the wait queue, one or more medical providers can see the patient in a queuing application based upon configurable patient filtering parameters. A medical provider can then optionally start a telehealth visit directly from the queuing application with a “ready” patient. If wait times exceed a configurable limit, an alarm can be designed to optionally be initiated to alert medical providers of available patients. Patients and providers may optionally use a time slot reservation system in tandem with the wait queue using an auto insert algorithm which inserts a patient into the queue at an appropriate time to meet the reserved time slot. This system has several non-limiting advantages. For example, patients who check in to a remotely located healthcare kiosk are automatically placed into a wait queue. Also, medical providers can optionally see patients who are checked into each healthcare kiosk based on the provider's access credentials. Furthermore, patients can optionally be filtered from provider view based upon their location, insurance, medical condition, age, and/or service desired. Also, medical providers can optionally have a profile of patient types they are eligible to see which assists in the filtering of patients in the wait queue. Furthermore, multiple medical providers can optionally see patient status as checked in, and/or in a ready state along with a patient wait timer. Also, patients are optionally sorted by wait time in ready state to make it easy for the provider to find the next available patient with the longest wait time. Still further, medical providers can optionally view patient medical history prior to a visit starting if the patient has elected to share their patient medical history with the unknown provider. Also, a configurable alarm when a patient is ready to be seen can optionally sound, text, and/or email a medical provider regarding the patient status. Also, a configurable alarm for a system administrator can optionally sound if the service level has passed a configurable limit. Furthermore, patients can optionally receive an estimated wait time based on an algorithm of past average wait time data in relation to the number of patients ahead in the queue for the given healthcare kiosk. Also, patients can optionally reserve a queue “slot” for a set time in the future at a given medical kiosk online, on a mobile device, and/or at the healthcare kiosk. Furthermore, when the wait queue at the medical kiosk reaches the time where the patient with a queue slot should be inserted into the queue (according to an algorithm which can estimate the appropriate insertion time), the patient can optionally be automatically inserted into the queue in order to be ready at the appointed reservation slot time. Also, the wait queue for a given medical kiosk, or network of kiosks can optionally be modified by an administrator to accommodate no-shows, or late patients/medical providers. Furthermore, medical providers can optionally schedule a queue slot for a given patient at a given medical kiosk. The reservation system of the present invention can be used to automatically insert the patient at the appointed time to meet the reservation. Using the system, method and apparatus of the present invention, the specific scheduling provider can be the only provider who is able to see the given patient in the queue; however, this is not required. In prior art health care queuing systems, the patient selects a specific provider in advance of the visit. In accordance with the present invention, the medical provider may not be known in advance to the patient, thus the scheduling system allow for a more efficient scheduling of appointments. Prior art systems do not deal with the complexity of scheduling a multitude of medical kiosks with specific locations with various network parameters, across a multitude of providers. The queuing method of the present invention enables the maximum throughput of patients at a medical kiosk. The prior art systems do not instruct on the auto insertion of a patient in a queue based on anticipated visit time. A system, method and apparatus of the present invention can streamline, speed up, and/or reduce wasted time for scheduling tele-medicine patient-provider visits in multitude of remotely located (or home/mobile based) medical kiosks by automating aspects that assist medical providers in managing waiting queues of patients.

In another and/or alternative non-limiting aspect of the invention, the novel method and apparatus for providing medical services, diagnoses, health, and/or wellness advice to individuals can include software that can be loaded into existing software (e.g., EPIC, etc.) used by a medical provider, hospital, insurance company, medical group, pharmacy, etc. so as to create a portal, link, etc. to the software used to perform the tele-med services of the present invention. The loaded software could be designed to create a widget on the screen of the medical provider, hospital, insurance company, medical group, pharmacy, etc. and the clicking of the widget activates the tele-med software on the computer and/or accesses another location to enable the user to perform the tele-med services, access records, view appointments, etc.

In yet another and/or alternative embodiment of the invention, the novel method and apparatus for providing medical services, diagnoses, health, and/or wellness advice to individuals can include a medical kiosk that includes a medical device panel and associated software to aggregate a plurality of medical devices into a single medical device panel and software system; however, this is not required. In the past, getting multiple manufacturers' APIs for controllable medical devices approved to work within an MDDS has proved challenging. Medical devices require FDA clearance to be used. Integrating multiple devices from various manufacturers can create significant challenges for a system integrator or solution provider that is trying to provide a good user experience and remain FDA compliant. In most cases, the manufacturer's API does not conform to the integrators needs. In accordance with one non-limiting embodiment of the invention, there is provided a system, method and apparatus that can create a standalone FDA- or other type of agency-approvable medical device control panel with a configurable user interface to adapt and harmonize with related software solutions, to interface directly with multiple third-party manufacturer's device integration APIs, and/or to reside as a completely separate module within a larger telehealth software solution. In one non-limiting aspect of the present invention, the medical device control panel software is a separate and standalone solution that can be seamlessly incorporated into a larger software solution. A system integrator or solution provider can use the control panel to aggregate multiple medical device APIs into a single medical device control software (with modular aspects for various devices) with configurable user interface, to keep all aspects of device control separate from any other software, limiting the FDA or other agency's approval process to this single piece of software. Another and/or alternative non-limiting aspect of the present invention is to create a development kit for device manufacturers to create medical device APIs within the architecture of this solution so that configurable medical device controls can easily be shared with various system integrators and the FDA or other agency's approval remains with the original device manufacturer. In another non-limiting aspect of the present invention is the ability of a medical provider to optionally remotely control a device through the medical device control panel using an FDA cleared method. In addition and/or alternatively, it is possible for the patient to also or alternatively optionally controls the medical device through the medical device control panel. Still another and/or alternative non-limiting aspect of the present invention is to optionally send the device data from the control panel directly to an EMR and/or PHR. Currently, medical devices that are integrated within a software, wherein the software user interface for device controls has been modified from those originally provided by the device manufacturer, require that the entire software is regulated by the FDA or other government agency. The present invention separates the medical device(s) from the overall software solution and allows for a configurable user interface across multiple medical devices, thus eliminating the need to change code in the main non-device software applications, and streamlining FDA or other agency regulatory concerns. A development toolkit for device manufacturers can optionally be provided that can be used to create medical device control “widgets” that are reusable and configurable for various system integrators so that the original device manufacturer retains FDA approval or ownership of the device. The segmentation of devices requiring various levels of FDA or other agency regulation can coexist in the same control panel. A software solution provider can be able to have configuration control of the user interface of a medical device manufacturer's software. The medical device control panel solution can be designed to reside in the provider application and the patient application which can be managed locally or in the cloud, separate from the main telehealth application. The present invention makes it much easier to gain FDA or other agency clearance for FDA or other agency medical devices where the system integrator or solution provider is not the manufacturer of the device. The FDA or other agency controlled aspects of the software can be kept within a single or modular separate software sharing a common medical device control architecture. The user interface can be controllable by the medical device control panel software component within the total solution without impacting the FDA or other agency clearance of the devices. If the device manufacturer uses the development kit, the medical device API and user interface can be easily reused by multiple solution providers with this invention. FDA or other agency clearance and registration can optionally become streamlined for medical device integration of companies using this solution. The user interface and usability of devices can be improved by use of the present invention. The medical device control panel can optionally be battery and/or USB powered for maximum portability or minimum complexity. The present invention is directed to a system, method and apparatus that can create a standalone FDA or other type of agency approvable medical device control panel with a configurable user interface to adapt and harmonize with related software solutions, to interface directly with multiple third-party manufacturer's device integration API's, and to reside as a completely separate module within a larger telehealth software solution.

In still another and/or alternative embodiment of the invention, the novel method and apparatus for providing medical services, diagnoses, health, and/or wellness advice to individuals can include a personal medical kiosk that can be used with one or more medical devices. In one non-limiting aspect of the invention, there is provided a personal medical kiosk that can be used with a plurality of medical devices that can be contained in a common housing. The multipurpose home and/or mobile medical device is designed to be simple to use. Most medical devices that are available are single purpose devices that are expensive, and are not necessarily intuitive for a casual or lay user. Prior art tele-medicine technologies have attempted to bring a healthcare visit into the home, or mobile device. The problem with this type of visit is that the medical provider may not be able to gather enough information about the patient's condition with just a videoconference. As a result, many medical device manufacturers have begun developing and selling medical devices that can be connected to a mobile device or to a home PC. These devices are commonly developed as “point solutions”, meaning they attempt to solve the problem from the device manufacturer's point of view—using a single device and the device software to connect to an often proprietary physician group—rather than having a mass market approach where the device can become part of another software solution. One major reason that the market has developed this way is because of the difficulties in FDA regulation requiring telehealth technology companies to solve complicated regulatory challenges. In one non-limiting aspect of the invention, there is provided a multipurpose home and/or mobile medical device that combines a plurality of medical devices into a common housing. The multipurpose home and/or mobile medical device is designed to be simple to use. The multipurpose home and/or mobile medical device is designed to leverage electronics, communications/connectivity. The multipurpose home and/or mobile medical device can also be designed to leverage FDA regulatory clearance of the medical devices. The multipurpose home and/or mobile medical device can connect via one or more known interfaces such as, but not limited to, USB, HDMI, Firewire, Bluetooth, IR, wireless technologies, etc. In one non-limiting arrangement, the multipurpose home and/or mobile medical device includes some combination or subset of at least two of the following devices: stethoscope, otoscope, dermascope, thermometer, blood pressure, pulse oximeter, ECG, spirometer, and glucometer. The multipurpose home and/or mobile medical device can be designed to collect vital information and/or gather images so that a remotely located healthcare provider can be provided with such collected information and make a sound and informed diagnoses of various conditions. The multipurpose home and/or mobile medical device can be designed to be simple enough for a patient to use on themselves in a consultation with a provider. The data from the multipurpose home and/or mobile medical device can optionally be shared real time, near real time, and/or asynchronously. The multipurpose home and/or mobile medical device can optionally be used by the patient to chronicle their vitals and/or capture images of the progress of their condition. The multipurpose home and/or mobile medical device can optionally include one or more methods for collecting data in a single housing. The patient (user) typically pairs their multipurpose home and/or mobile medical device with a mobile device (e.g., smart phones, tablets, laptop computers, notepads, etc.), home PC, and/or entertainment console (e.g., Xbox systems, PS3, PS4, etc.), or any other device that can connect to a phone, satellite and/or internet and which allows the multipurpose home and/or mobile medical device to provide data to a medical device software residing on one of these technologies and which controls the display and routing of the data. Some aspects of the multipurpose home and/or mobile medical device can optionally be remotely controlled by the healthcare provider. The medical device of the present invention combine multiple devices into a single unit for providing the most commonly needed data points for a telehealth medical visit. The medical devices can streamline electronics, connectivity and mechanical properties, as well as FDA or other agency registration, by sharing services and capabilities as much as possible. The device will minimize the space needed to store all the devices. The medical device of the present invention can serve more than one purpose while the prior art is single purpose built. The device of the present invention enables one time set-up for connectivity while the prior art requires connectivity for multiple devices. The device of the present invention can require a single FDA or other agency registration while the prior art requires FDA or other agency registration for multiple devices. The device of the present invention can leverage a medical device control panel software to maximize flexibility, minimize device costs and maximize user experience, while simplifying FDA or other agency regulatory matters. This invention can potentially dramatically reduce the cost of having a home telehealth visit and can also or alternatively potentially dramatically improve the quality of the visit.

In yet another and/or alternative embodiment of the invention, the novel method and apparatus for providing medical services, diagnoses, health, and/or wellness advice to individuals can enable a patient to create, own, access and/or enable third parties to access and/or modify the patient's own Patient Health Record (PHR). Sharing patient data to healthcare provider networks has created some interesting challenges. The traditional model is for a patient to have a HIPAA-based privacy practices relationship with a Covered Entity. This allows the Covered Entity (provider) to use patient data with patient permission. It also may allow a Covered Entity to share information with a business associate. The patient data in a HIPAA relationship is owned by the provider. It may be difficult for a patient to gain access to their data and to share it with other providers outside the Covered Entity network. A relatively new model for storing patient data has been developed called a Personal Health Record (PHR). A PHR puts the patient in charge of their electronic health data, allowing them to retain ownership and share their health data with individuals and providers of their choosing. These PHR systems have typically been website repositories where patients input their PHR information. In one non-limiting embodiment, there is provided a system, method and apparatus that allows a patient to create, modify, own, access and/or enable third parties to access and/or modify the patient's own PHR record at a medical kiosk and/or at some other location or device (e.g., home PC, mobile device, entertainment center, or any other device that can access the phone, satellite and/or internet, etc.). The patient can optionally choose to share the PHR with a medical provider and/or commence a tele-medicine visit all in one convenient location. Medical devices with the medical kiosk and/or other location can send data directly to the PHR, storing it securely and optionally allowing immediate retrieval by an authorized provider during a visit. The remotely located medical provider can optionally have real time or near real time access to the PHR data, including medical device data from the medical kiosk and/or at some other location. The medical provider can optionally be allowed to update the patient's PHR data directly during a visit. The patient can have access to the updated PHR data from any phone, satellite, internet and/or intranet enabled device. The medical provider may also optionally be allowed by the patient to preserve a copy of the healthcare visit through integration with the medical provider's own EMR system. In accordance with one aspect of the present invention, the patient is able to keep their medical history information in a convenient, secure repository. The patient can share their data with various individuals, including medical providers. The patient inputs their healthcare information on a website, or directly into the medical kiosk or at some other location (e.g., home PC, mobile device, entertainment center, etc.). The creation of a PHR account and data at a remotely located, private, medical kiosk and/or some other location (e.g., home PC, mobile device, entertainment center, etc.) with medical devices where a tele-medicine visit can occur can be advantageous and convenient to a patient and/or medical provider. Another non-limiting advantage of the invention is the ability to create PHR data on a website, and later update and review that data, or simply access and share the PHR created on the website with a provider seen in the medical kiosk and/or some other location (e.g., home PC, mobile device, entertainment center, etc.). Another non-limiting advantage of the invention is the ability of a medical device at a remote medical station to send data immediately to the PHR datastore, and to be retrieved immediately by a remotely located provider. Another non-limiting advantage of the invention is the ability of a medical provider to immediately receive PHR data from a patient at a remotely located medical kiosk and/or some other location (e.g., home PC, mobile device, entertainment center, etc.) to conduct a real time or near real time healthcare consultation. Another, non-limiting advantage of the invention is the ability of a medical provider to gather PHR data from the medical kiosk and/or some other location (e.g., home PC, mobile device, entertainment center, etc.) consultation directly into the medical provider's own EMR system given a method to gain the express permission of the patient. Another non-limiting advantage of the invention is the ability of historical medical data, and/or medical data being created from a medical provider's EMR for a patient being seen in a medical kiosk and/or some other location (e.g., home PC, mobile device, entertainment center, etc.) then can be automatically transported to that patient's PHR during or after the visit. Another non-limiting advantage of the invention is the ability to enable patient access at a later time to healthcare data generated at the medical kiosk and/or some other location (e.g., home PC, mobile device, entertainment center, etc.) via a secure website. Another non-limiting advantage of the invention is the ability of a different medical provider, if given permission by the patient, to access the patient history created by one or more other medical providers without needing to contact those other medical providers. Another non-limiting advantage of the invention is the ability to create a PHR account at a remote medical kiosk location and/or some other location (e.g., home PC, mobile device, entertainment center, etc.) and use it immediately to consult with a medical provider. Another non-limiting advantage of the invention is the ability to create PHR data on a website and later use it at a remote medical kiosk and/or some other location (e.g., home PC, mobile device, entertainment center, etc.). Another non-limiting advantage of the invention is the ability for the patient to maintain ownership of their health information and share their healthcare data created at the medical kiosk and/or some other location (e.g., home PC, mobile device, entertainment center, etc.) with any medical provider or individual of their choosing. Another non-limiting advantage of the invention is the ability to have better continuity of care and speed of information sharing.

In yet another and/or alternative embodiment of the invention, the novel method and apparatus for providing medical services, diagnoses, health, and/or wellness advice to individuals there is provided a medical kit that includes one or more medical devices that can be used with a personal medical kiosk. In one non-limiting configuration of the medical kit, there is provided a container that has a base that includes a plurality of compartments for holding one or more medical devices. The one or more compartments can also contain other materials and/or devices (e.g., band-aid, bandage, gauze, tweezers, scissors, etc.). The container can be made of various types of materials (e.g., plastic, metal, etc.) The shape, size and configuration of the container are non-limiting. The container can be shaped and size to be portable so that it can be conveniently conveyed and/or used in a variety of locations. The lid of the container can include a compartment that is designed to removably hold a tablet computer, smart device, smart phone, IPad, or the like. As can be appreciated, the lid of the container can incorporate a permanent display. If a tablet computer, IPad, or the like is used with the container, the tablet computer, IPad, or the like is used as the display for the personal medical kiosk. The camera on the tablet computer, IPad, or the like is used to convey images to the remotely located medical provider. Software can be loaded into the tablet computer, IPad, or the like to interface with the remotely located medical provider, and/or the container can include hardware and/or software that is used with the tablet computer, IPad, or the like. The one or more medical devices can be wirelessly connected or directly to the hardware and/or software in the container, and/or be wirelessly connected or directly to the tablet computer, IPad, or the like. The container can include a lock or other security arrangement to inhibit or prevent unauthorized use of the container. The container can include one or more connectors (e.g., USB, Bluetooth, Wi-Fi, etc.).

It is one non-limiting object of the invention to provide tele-medicine services that are convenient to a user.

It is another and/or alternative one non-limiting object of the invention to provide tele-medicine services that are cost effective to a user.

It is still another and/or alternative one non-limiting object of the invention to provide tele-medicine services that can be provided to a user via a medical kiosk.

It is yet another and/or alternative one non-limiting object of the invention to provide tele-medicine services that can be provided to a user via a modular medical kiosk.

It is still yet another and/or alternative one non-limiting object of the invention to provide a medical kiosk and method for using a medical kiosk that is easy to assemble and disassemble.

It is another and/or alternative one non-limiting object of the invention to provide a medical kiosk and method for using a medical kiosk that includes an easy and convenient registration system and payment system.

It is still another and/or alternative one non-limiting object of the invention to provide a medical kiosk and method for using a medical kiosk that provides privacy to a user when obtaining medical services.

It is yet another and/or alternative one non-limiting object of the invention to provide a medical kiosk and method for using a medical kiosk that is sized and shaped to accommodate disabled or handicapped users.

It is still yet another and/or alternative one non-limiting object of the invention is to provide a medical kiosk and method for using a medical kiosk that includes medical instruments that can be used by a user when obtaining medical services.

It is another and/or alternative one non-limiting object of the invention to provide a medical kiosk and method for using a medical kiosk that includes video conferencing capabilities between a user and a medical provider.

It is still another and/or alternative one non-limiting object of the invention to provide a medical kiosk and method for using a medical kiosk that provides the option to play back one or more portions of the video conference session to the user after the video conference between the user and medical provider has been completed.

It is yet another and/or alternative one non-limiting object of the invention to provide a medical kiosk and method for using a medical kiosk that includes an attendant station, digital signage, and/or a registration station on the exterior of the medical kiosk.

It is still yet another and/or alternative one non-limiting object of the invention to provide a medical kiosk and method for using a medical kiosk that includes a Provider Screen and/or Patient Screen in the interior of the medical kiosk.

It is another and/or alternative one non-limiting object of the invention to provide a medical kiosk and method for using a medical kiosk that includes one or more medical device cabinets on the interior of the medical kiosk.

It is still another and/or alternative one non-limiting object of the invention to provide a medical kiosk and method for using a medical kiosk that includes the use of a Patient Appointment or Registration Application, Attendant Application, Patient Appointment or Registration Application, Provider Application, Administrator Application, Patient Portal and/or Provider Portal.

It is yet another and/or alternative one non-limiting object of the invention to provide a medical kiosk and method for using a medical kiosk that includes a Patient Appointment or Registration Application that enables patients to schedule an appointment at a medical kiosk.

It is still yet another and/or alternative one non-limiting object of the invention to provide a medical kiosk and method for using a medical kiosk that includes Patient Appointment or Registration Application that enables a patient to enter insurance information and/or to make a payment and/or copay for the medical visit in the kiosk.

It is another and/or alternative one non-limiting object of the invention to provide a medical kiosk and method for using a medical kiosk that includes Patient Registration System that enables a patient to check-in for a preexisting appointment.

It is still another and/or alternative one non-limiting object of the invention to provide a medical kiosk and method for using a medical kiosk that includes a Patient Registration System that enables a patient to identify the patient's symptoms, medications, allergies, and/or medical conditions.

It is yet another and/or alternative one non-limiting object of the invention to provide a medical kiosk and method for using a medical kiosk that includes registration and/or check-in system that enables a patient to request assistance from a medical attendant.

It is still yet another and/or alternative one non-limiting object of the invention to provide a medical kiosk and method for using a medical kiosk that includes an Attendant Application that enables a medical attendant to monitor past, present and/or future appointments for the medical kiosk.

It is another and/or alternative one non-limiting object of the invention to provide a medical kiosk and method for using a medical kiosk that includes an Attendant Application that enables a medical attendant to monitor the status of a current visit in the medical kiosk.

It is still another and/or alternative one non-limiting object of the invention to provide a medical kiosk and method for using a medical kiosk that includes an Attendant Application that enables a medical attendant to cancel and/or reschedule an appointment to a medical kiosk.

It is yet another and/or alternative one non-limiting object of the invention to provide a medical kiosk and method for using a medical kiosk that includes an Attendant Application that informs that medical attendant that the patient requires assistance.

It is still yet another and/or alternative one non-limiting object of the invention to provide a medical kiosk and method for using a medical kiosk that includes an Attendant Application that enables the medical attendant to keep track of the clean-up of the medical kiosk.

It is another and/or alternative one non-limiting object of the invention to provide a medical kiosk and method for using a medical kiosk that enables the medical assistant to activate an automated sanitation and/or cleaning system of the medical kiosk.

It is still another and/or alternative one non-limiting object of the invention to provide a medical kiosk and method for using a medical kiosk that includes an Attendant Application that enables a medical assistant to validate the insurance and/or ID of a patient.

It is yet another and/or alternative one non-limiting object of the invention to provide a medical kiosk and method for using a medical kiosk that includes a Patient Appointment or

Registration Application that helps the patient capture vitals and/or enter the vitals information during the visit.

It is still yet another and/or alternative one non-limiting object of the invention to provide a medical kiosk and method for using a medical kiosk that includes a Patient Appointment or Registration Application that enables the patient to request assistance from the medical attendant.

It is another and/or alternative one non-limiting object of the invention to provide a medical kiosk and method for using a medical kiosk that includes a Patient Appointment or Registration Application that can assist the patient during the consultation with the medical provider.

It is still another and/or alternative one non-limiting object of the invention to provide a medical kiosk and method for using a medical kiosk that includes a Patient Appointment or Registration Application that enables a patient to take a survey regarding the consultation with the medical provider and/or the use of the medical kiosk.

It is yet another and/or alternative one non-limiting object of the invention to provide a medical kiosk and method for using a medical kiosk that includes a Provider Application that assists the medical provider in providing medical services to the patient in the medical kiosk.

It is still yet another and/or alternative one non-limiting object of the invention to provide a medical kiosk and method for using a medical kiosk that includes a Provider Application that provides information to the medical provider regarding past, current and future appointment that have been scheduled with the medical provider.

It is another and/or alternative one non-limiting object of the invention to provide a medical kiosk and method for using a medical kiosk that includes a Provider Application that enables the medical provider to view personal information that the patient has entered regarding the medical visit.

It is still another and/or alternative one non-limiting object of the invention to provide a medical kiosk and method for using a medical kiosk that includes a Provider Application that enables the medical provider to view vitals that have been collected on the patient.

It is yet another and/or alternative one non-limiting object of the invention to provide a medical kiosk and method for using a medical kiosk that includes a Provider Application that enables the medical provider to request that the medical attendant assist the patient in the medical kiosk.

It is still yet another and/or alternative one non-limiting object of the invention to provide a medical kiosk and method for using a medical kiosk that includes a Provider Application that enables the medical provider to control the use and/or access of one or more medical devices in the medical kiosk.

It is another and/or alternative one non-limiting object of the invention to provide a medical kiosk and method for using a medical kiosk that includes a Provider Application that enables a medical provider to create a visit summary of the patient in the medical kiosk.

It is still another and/or alternative one non-limiting object of the invention to provide a medical kiosk and method for using a medical kiosk that includes a Provider Application that enables the medical provider to update personal information, medical history information and/or symptom information about the patient.

It is yet another and/or alternative one non-limiting object of the invention to provide a medical kiosk and method for using a medical kiosk that includes a Provider Application that enables a medical provider to select and/or enter a diagnosis for a patient.

It is still yet another and/or alternative one non-limiting object of the invention to provide a medical kiosk and method for using a medical kiosk that includes a Provider Application that enables the medical provider to enter notes about a patient and/or create a treatment plan for a patient.

It is another and/or alternative one non-limiting object of the invention to provide a medical kiosk and method for using a medical kiosk that includes a Provider Application that enable a medical provider to view information about past visits by a patient.

It is still another and/or alternative one non-limiting object of the invention to provide a medical kiosk and method for using a medical kiosk that includes a mobile application and/or an on-line application that enables a patient to 1) conveniently locate an available medical kiosk, 2) schedule an appointment (e.g., date and/or time), 3) pre-register symptoms and/or reasons for visit, 4) set and/or cancel an appointment, 5) receive reminders and/or updates regarding appoints, 6) obtain information about medical kiosk availability, 7) obtain information about certain medical provider availability, 8) obtain information about the available medical provider (e.g., name, specialty, etc.), 9) enable the selection of a certain medical provider and/or medical provider in a certain field of medicine, 10) obtain map information, address information and/or hours of operation information regarding selected medical kiosk, 11) locate closest kiosk and/or kiosk availability for a certain medical provider and/or medical provider in a certain field of medicine, 12) presubmit and/or preclear medical insurance, 13) submit payment information, 14) receive information on payment status, 15) receive information in insurance coverage, 16) receive appointment reminders and/or updates, and/or 17) receive medication reminders, and the like.

It is yet another and/or alternative one non-limiting object of the invention to provide a medical kiosk and method for using a medical kiosk that includes a scale in the floor of the medical kiosk.

It is still yet another and/or alternative one non-limiting object of the invention to provide a medical kiosk and method for using a medical kiosk that includes a medication adherence application that allows a user/patient to 1) speak to a pharmacist, 2) change the patient's medications alerts, 3) learn about certain types of medical conditions, 4) check medication orders status, 5) check medication delivery status, 6) refill a prescriptions, 7) transfer prescriptions to a another location, 8) obtain information about recommended medication dosages, 9) obtain information about recommended times to take medications, 10) obtain information about recommended frequency for taking medications, 11) obtain information about medications, 12) obtain information about generic brands available for medications, 13) request an appointment to speak with a medical provider, 14) enter information regarding compliance information regarding medication usage by patient, and/or 15) receive compliance reports for patients regarding medication usage.

It is another and/or alternative one non-limiting object of the invention to provide a medical kiosk and method for using a medical kiosk that includes a medication adherence application that 1) sends reminders for user/patients to take their medications, 2) monitors a user's/patient's adherence to taking their medications and/or distributes progress reports to the patient, and/or 3) automatically enrolls a patient in an electronic prescriptions network which can optionally send a patient's prescriptions to their choice of pharmacy.

It is still another and/or alternative one non-limiting object of the invention to provide a medical kiosk and method for using a medical kiosk that includes one or more headphone jacks in the medical kiosk.

It is yet another and/or alternative one non-limiting object of the invention to provide a medical kiosk and method for using a medical kiosk that includes a medical kiosk having HD components and an industrial strength design.

It is still yet another and/or alternative one non-limiting object of the invention to provide a medical kiosk and method for using a medical kiosk that includes a medical kiosk having a modular configuration and/or a configuration that enables the medical kiosk to be brought in to or removed from locations having a standard sized doorway.

It is another and/or alternative one non-limiting object of the invention to provide a medical kiosk and method for using a medical kiosk that includes a medical kiosk having a check-in registration system positioned on the exterior of the medical kiosk.

It is still another and/or alternative one non-limiting object of the invention to provide a medical kiosk and method for using a medical kiosk that includes a medical kiosk having one or more medical device cabinets located in the interior of the medical kiosk.

It is yet another and/or alternative one non-limiting object of the invention to provide a medical kiosk and method for using a medical kiosk that includes a medical kiosk having digital signage on the interior and/or exterior of the medical kiosk.

It is still yet another and/or alternative one non-limiting object of the invention to provide a medical kiosk and method for using a medical kiosk that includes a medical kiosk having a built-in sanitation system.

It is another and/or alternative one non-limiting object of the invention to provide a medical kiosk and method for using a medical kiosk that allows patients to 1) choose between name brand and generic drugs, 2) choose the supply quantity for the prescription (i.e., 30-day supply, 60-day supply, 90-day supply, etc.), 3) choose between picking the prescription up at the pharmacy of their choice or mail delivery of the prescription, 4) enter medical insurance for partial or full payment of the prescription, 5) enter a credit or debit card information to pay for the prescription, 6) enter information for mail delivery of the prescription, 7) enter information to provide automatic reminders to patient regarding refilled and/or follow-up medical visits, 8) enter information to enable patient to be notified when prescription has been mailed and/or is ready to be picked-up at the pharmacy; however, this is not required, 9) obtain a printout and/or electronic version of the prescription written by the medical provider, and/or 10) receive information about the issued prescription (e.g., prescribed use, side effects, etc.) in printout and/or electronic form.

It is still another and/or alternative one non-limiting object of the invention to provide a medical kiosk and method for using a medical kiosk that includes a medical kiosk having a movable front interior wall panel to enable access to components that are positioned behind the front interior wall of the medical kiosk.

It is yet another and/or alternative one non-limiting object of the invention to provide a medical kiosk and method for using a medical kiosk that includes a scheduling system for scheduling a patient for a tele-medicine visit into a remotely located healthcare kiosk.

It is still yet another and/or alternative one non-limiting object of the invention to provide a medical kiosk and method for using a medical kiosk that includes a scheduling system that can streamline, speed up, and/or reduce wasted time for scheduling tele-medicine patient-provider visits in multitude of remotely located (or home/mobile based) medical kiosks by automating aspects that assist medical providers in managing waiting queues of patients.

It is another and/or alternative one non-limiting object of the invention to provide a medical kiosk and method for using a medical kiosk that includes a scheduling system that can assist patients with information about expected wait times.

It is still another and/or alternative one non-limiting object of the invention to provide a medical kiosk and method for using a medical kiosk that includes a scheduling system that can use wait queuing and/or reservation scheduling for a medical kiosk.

It is yet another and/or alternative one non-limiting object of the invention to provide a medical kiosk and method for using a medical kiosk that includes a medical device panel and associated software to aggregate a plurality of medical devices into a single medical device panel and software system.

It is still yet another and/or alternative one non-limiting object of the invention to provide a medical kiosk and method for using a medical kiosk that uses a standalone FDA- or other type of agency-approvable medical device control panel with a configurable user interface to adapt and harmonize with related software solutions, to interface directly with multiple third-party manufacturer's device integration APIs, and/or to reside as a completely separate module within a larger telehealth software solution.

It is another and/or alternative one non-limiting object of the invention to provide a medical kiosk and method for using a medical kiosk that includes medical device control panel software that is a separate and standalone solution that can be seamlessly incorporated into a larger software solution.

It is still another and/or alternative one non-limiting object of the invention to provide a medical kiosk and method for using a medical kiosk that includes a medical device control panel software to aggregate multiple medical device APIs into a single medical device control software (with modular aspects for various devices) with configurable user interface, to keep all aspects of device control separate from any other software, limiting the FDA or other agency's approval process to this single piece of software.

It is yet another and/or alternative one non-limiting object of the invention to provide a medical kiosk and method for using a medical kiosk that includes a development kit for device manufacturers to create medical device APIs within the architecture of this solution so that configurable medical device controls can easily be shared with various system integrators and the FDA or other agency's approval remains with the original device manufacturer.

It is still yet another and/or alternative one non-limiting object of the invention to provide a medical kiosk and method for using a medical kiosk that includes the ability of a medical provider to optionally remotely control a device through the medical device control panel using an FDA cleared method.

It is another and/or alternative one non-limiting object of the invention to provide a medical kiosk and method for using a medical kiosk that allows the device data from the control panel to be sent directly to an EMR and/or PHR.

It is still another and/or alternative one non-limiting object of the invention to provide a medical kiosk and method for using a medical kiosk that includes a personal medical kiosk that can be used with a plurality of medical devices that can be contained in a common housing.

It is yet another and/or alternative one non-limiting object of the invention to provide a medical kiosk and method for using a medical kiosk that includes a multipurpose home and/or mobile medical device that includes some combination or subset of at least two of the following devices: stethoscope, otoscope, dermascope, thermometer, blood pressure, pulse oximeter, ECG, spirometer, and glucometer.

It is still yet another and/or alternative one non-limiting object of the invention to provide a medical kiosk and method for using a medical kiosk that enables a patient to create, modify, own, access and/or enable third parties to access and/or modify the patient's own PHR record at a medical kiosk and/or at some other location or device (e.g., home PC, mobile device, entertainment center, or any other device that can access the phone, satellite and/or internet, etc.).

It is yet another and/or alternative one non-limiting object of the invention to provide a medical kiosk and method for using a medical kiosk that enables a medical provider to be allowed to update the patient's PHR data directly during a visit.

It is still yet another and/or alternative one non-limiting object of the invention to provide a medical kiosk and method for a personal medical kiosk that includes the use of a medical kit that includes one or more medical devices that can be used with a personal medical kiosk.

It is another and/or alternative one non-limiting object of the invention to provide a medical kiosk and method for using a personal medical kiosk that includes the use of a medical kit that includes a plurality of compartments for holding one or more medical devices using a personal medical kiosk that includes the use of a medical kit and optionally includes one or more compartments that can contain other materials and/or devices (e.g., band-aid, bandage, gauze, tweezers, scissors, etc.).

These and other objects and advantages will become apparent to those skilled in the art upon reading and following the description taken together with the accompanying drawings.

### BRIEF DESCRIPTION OF THE DRAWINGS

Reference may now be made to the drawings which illustrate various preferred embodiments that the invention may take in physical form and in certain parts and arrangement of parts wherein:

FIG. 1 is an exploded view of a medical kiosk in accordance with the present invention;

FIG. 2 is a front elevation view of the medical kiosk of FIG. 1;

FIG. 3 is a top view of the medical kiosk of FIG. 1;

FIG. 4 is a bottom view of the medical kiosk of FIG. 1;

FIG. 5 is a front side view of the medical kiosk of FIG. 1;

FIG. 6 is a back side view of the medical kiosk of FIG. 1;

FIG. 7 is a back view of the medical kiosk of FIG. 1;

FIG. 8 is a top interior view of the medical kiosk of FIG. 1;

FIG. 9 is a top interior view of the medical kiosk of FIG. 1 that illustrates the front interior panel in the open position;

FIG. 10 is a cross-section view along line 10-10 of FIG. 8;

FIG. 11 is an enlarged view of the medical cabinets of FIG. 10;

FIG. 12 is a cross-section view along line 10-10 of FIG. 8 viewed from the opposite direction;

FIG. 13 is a front view of another embodiment of the medical kiosk;

FIG. 14 is a side view of a non-limiting embodiment of a multipurpose home and/or mobile medical device that includes a plurality of medical devices;

FIG. 15-18 are various non-limiting examples of displays for a medical device control panel; and,

FIG. 19 is a front elevation view of a non-limiting personal medical kiosk that is integrated with a medical device kit.

### DETAILED DESCRIPTION OF ONE NON-LIMITING EMBODIMENTS

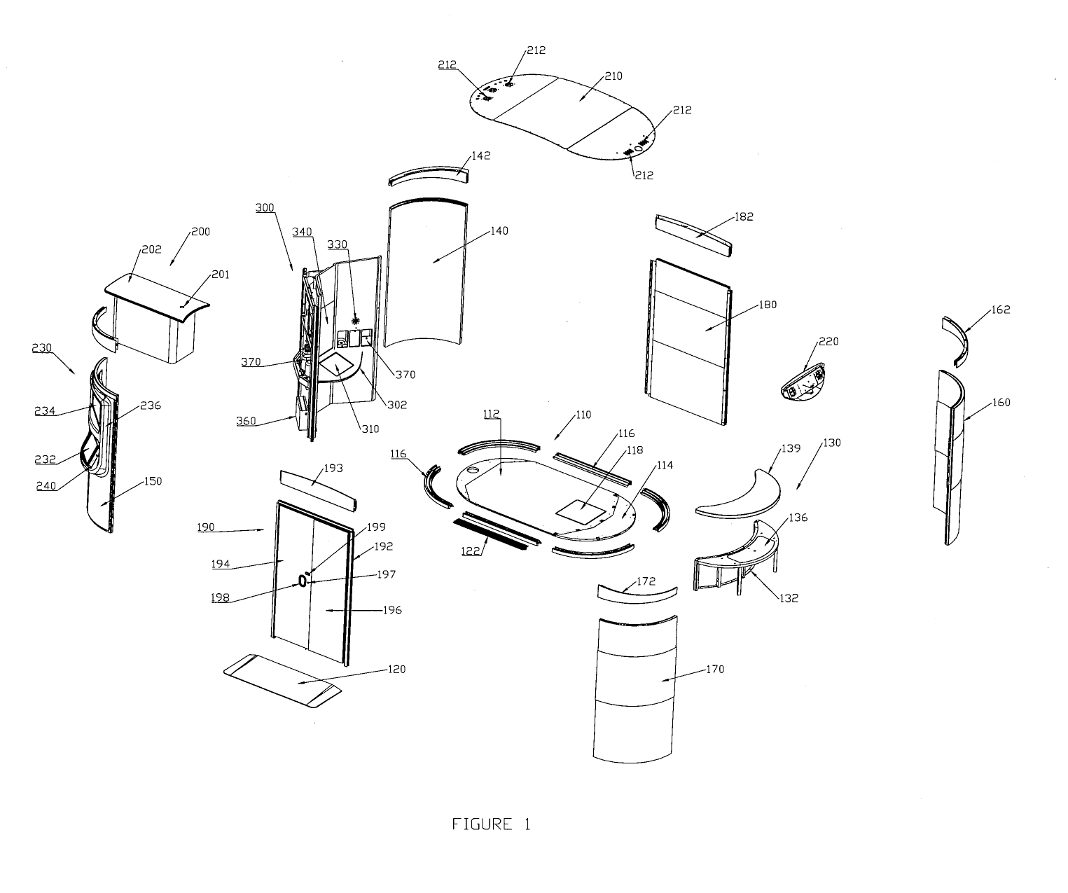

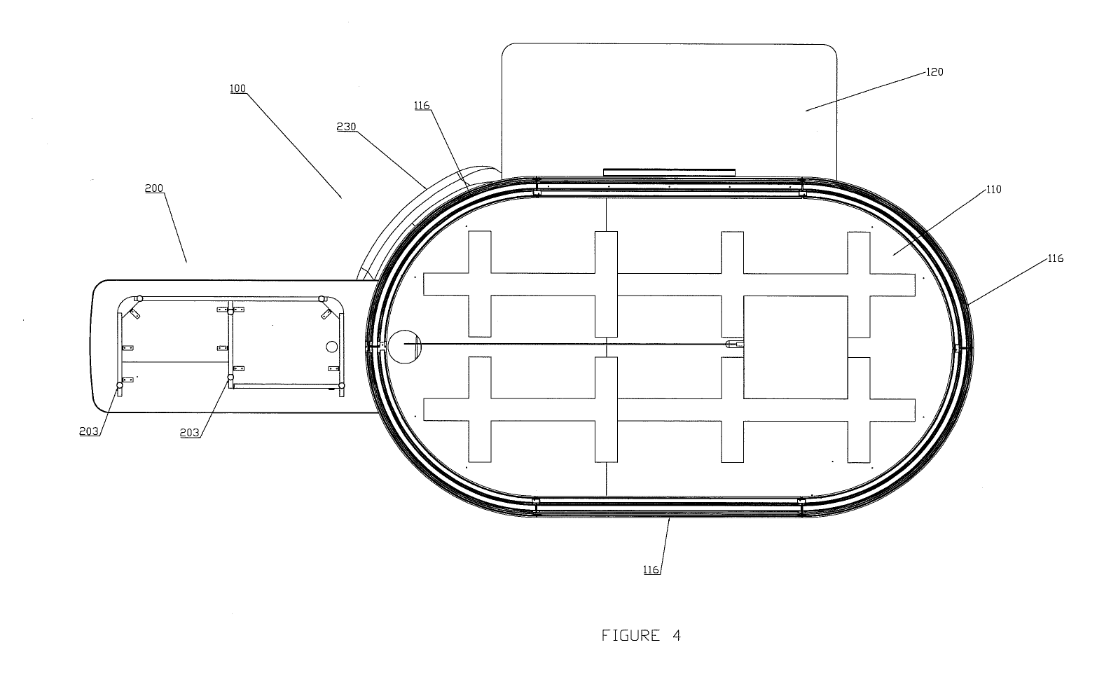

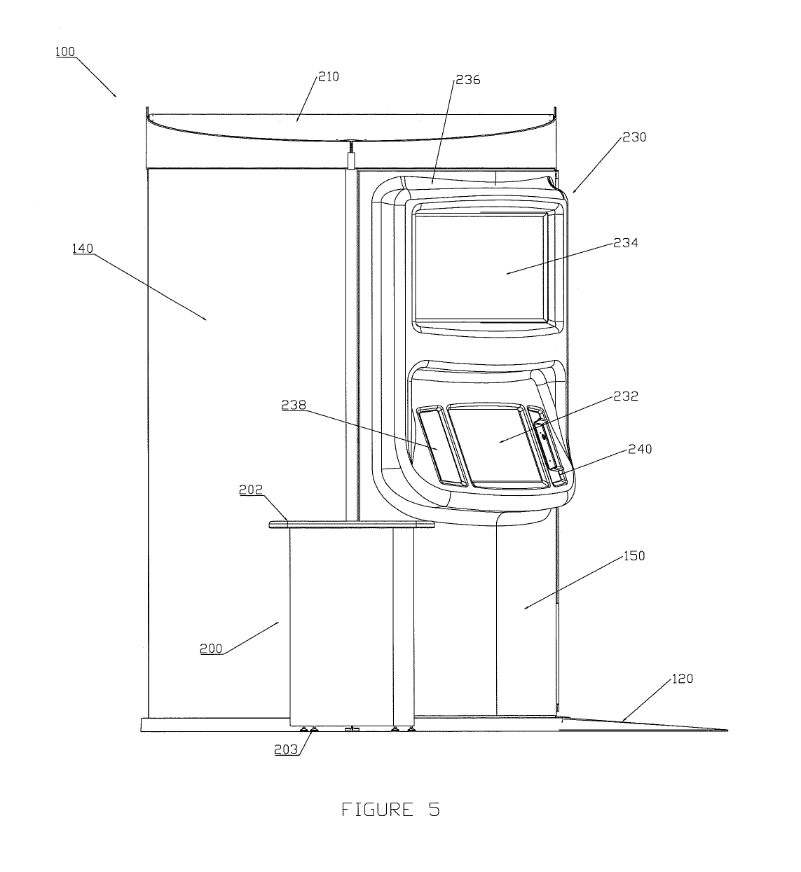

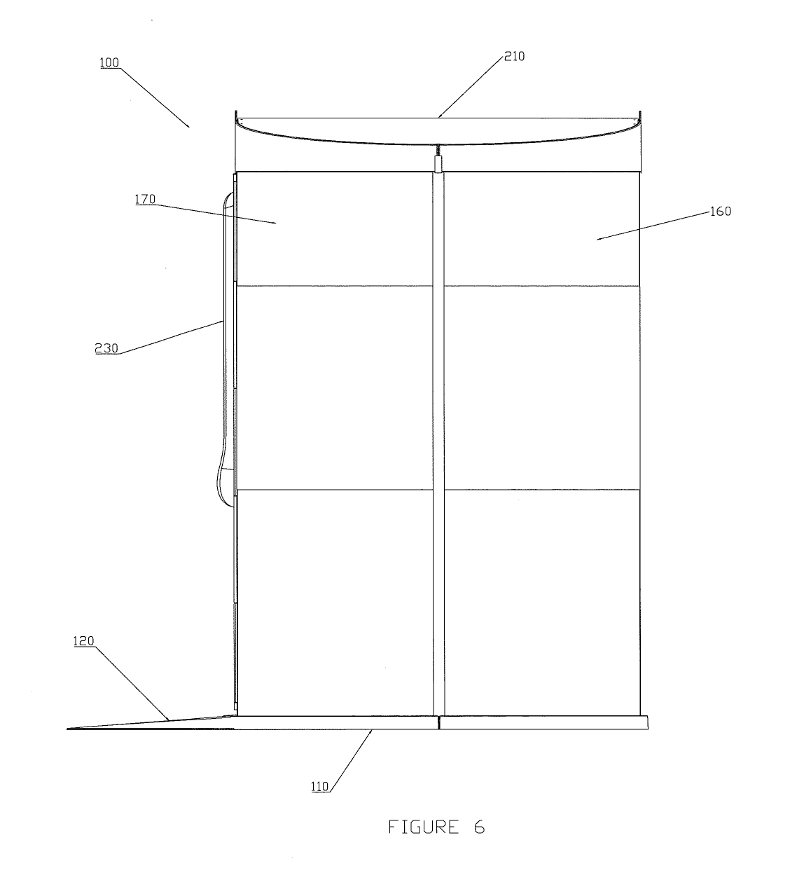

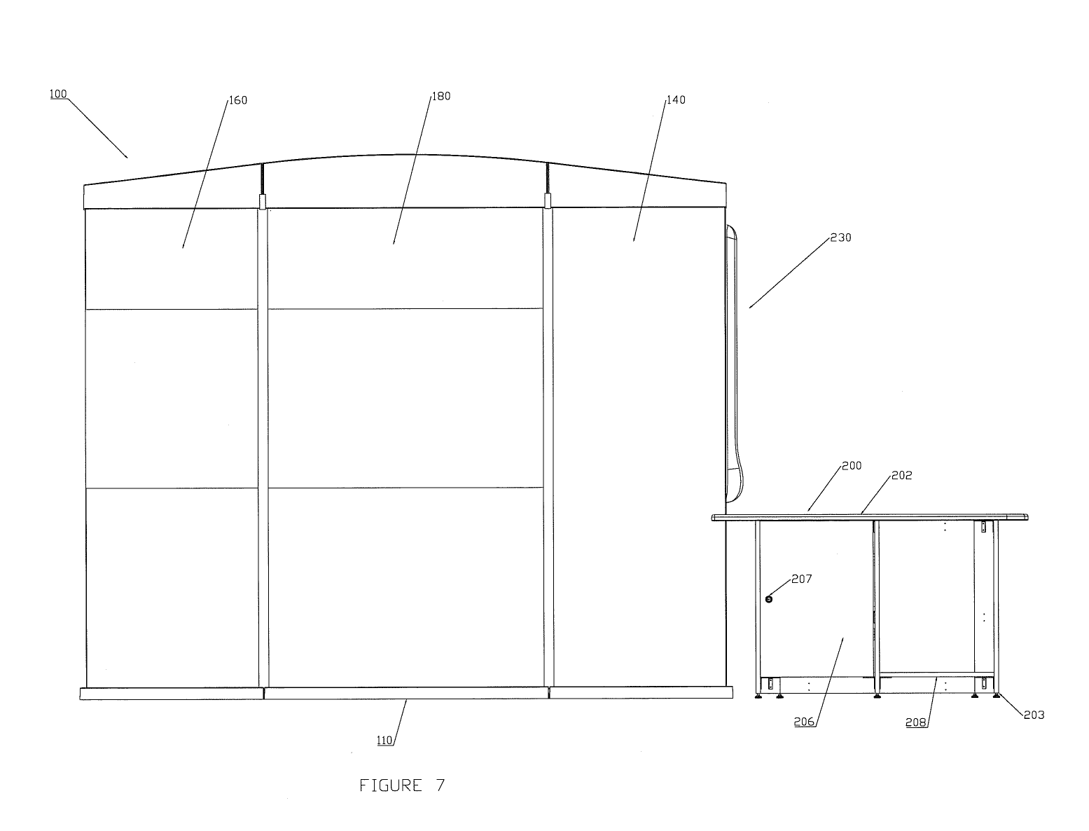

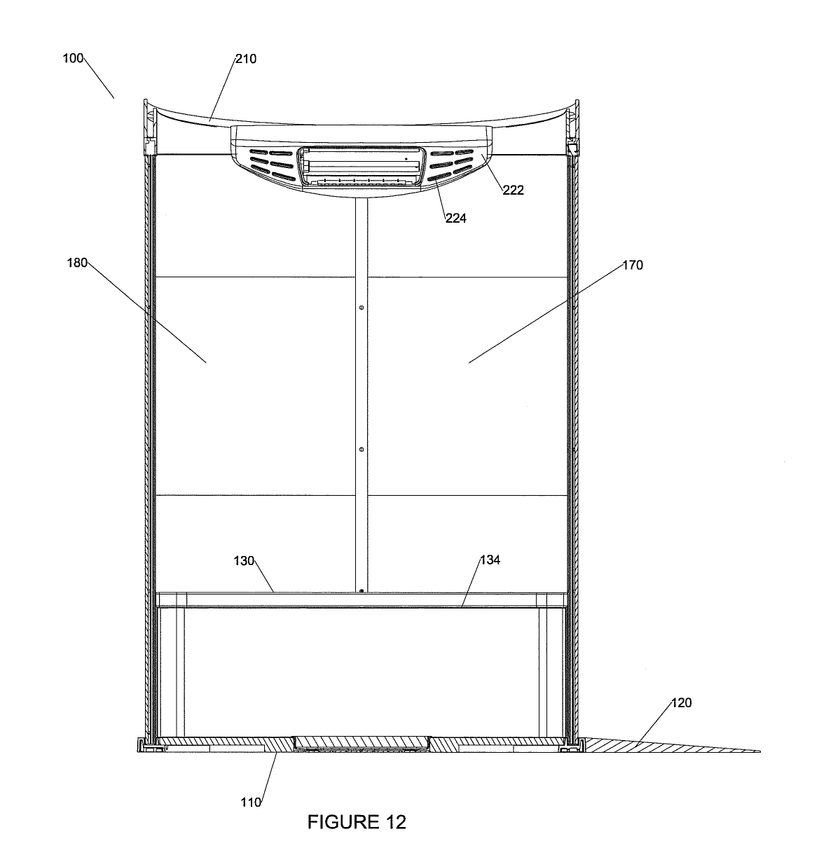

Referring now to the drawings wherein the showings are for the purpose of illustrating one non-limiting embodiments of the invention only and not for the purpose of limiting same, FIGS. 1-12 illustrate one non-limiting embodiment of the medical kiosk in accordance with the present invention. The medical kiosk 100 is designed to be used by a user to obtain medical services. Such medical services are generally tele-medicine services wherein one or more medical providers located at a remote location provide medical services to one or more users that are using the medical kiosk. As can be appreciated, non-tele-medicine services can also be provided to a user that is using the medical kiosk.

The shape, size and configuration of the medical kiosk are non-limiting. The materials and colors of the medical kiosk are also non-limiting. Generally, the materials used to form the medical kiosk include materials that resist or prevent microbial growth; however, this is not required. The medical kiosk illustrated in FIGS. 1-12 is designed to accommodate about 1-4 adults; however, it can be appreciated that the medical kiosk can be designed to accommodate additional users.

The medical kiosk is generally designed to be modular so that it can be easily assembled and disassembled; however, this is not required. FIG. 1 illustrates one non-limiting set of modular components of the medical kiosk. The medical kiosk generally includes a floor panel 110; however, this is not required. The floor panel, when used, can be a one or two piece unit; however, the floor panel can be formed of more than two pieces. As illustrated in FIG. 1, the floor panel is formed of two pieces 112, 114 that can be positioned and/or connected together in a variety of ways. A rail system 116 can be positioned about the floor panel to facilitate in connecting other components of the medical kiosk to the floor panel; however, this is not required. The rail system, when used, can be connected to the floor panel and/or other components of the medical kiosk in a variety of ways. The floor panel is generally formed of a durable material (e.g., plastic, metal, wood, composite material, man-made materials, etc.). The floor panel can be formed of a slightly compressible material to facilitate in the comfort of walking on the floor panel; however, this is not required. The floor panel is illustrated as having an oval shape; however, other shapes can be used (e.g., circular, square, rectangular, polygonal, etc.). The maximum length of the floor panel is generally 3-15 feet, typically 4-12 feet, more typically about 6-10 feet, and even more typically about 8-9 feet; however, other lengths can be used. The maximum width of the floor panel is generally 3-10 feet, typically 4-8 feet, and more typically about 4-6 feet; however; other widths can be used. The top surface area of the floor panel is generally 10-150 sq. ft., typically 15-80 sq. ft., and more typically about 50-60 sq. ft.; however, other surface areas of the floor panel can be used. The floor panel can be sized to enable a user in a wheelchair to enter the medical kiosk and turn and/or fully maneuver in the medical kiosk while sitting in the wheelchair; however, this is not required. The thickness of the floor panel is generally about 0.1-5 inches, and typically about 0.25-3 inches; however, other thicknesses of the floor panel can be used.

A weight scale 118 can optionally be partially or fully embedded in the floor panel. As can be appreciated, a weight scale can be placed on the top surface of the floor panel and/or positioned on other regions of the medical kiosk (e.g., chair, bench, etc.). The weight scale, when used, provides information about the weight of a user. The information from the scale can be electronically (e.g., wired, wirelessly) transferred to a medical provider and/or displayed to the user and/or medical provider.

A ramp 120 can be optionally used to facilitate entry and exiting of the medical kiosk; however, this is not required. The shape and size of the ramp are non-limiting. The ramp can be made of a similar material as the floor panel; however, this is not required. The ramp generally includes a sloped surface to facilitate in transitioning from a floor surface to the top surface of the floor panel; however, this is not required. A ramp connector 122 can be used to connect the ramp to the floor panel; however, this is not required.

The medical kiosk can optionally include one or more benches 130, stools and/or chairs. When bench 130 is included in the medical kiosk, the bench is generally positioned on the back interior wall of the medical kiosk; however, this is not required. The bench can be used to allow a parent, guardian, spouse, relative, friend, etc. to sit in the medical kiosk while the user is obtaining medical services in the medical kiosk. The bench can be designed to enable one or more persons to sit on the bench. The bench can optionally include a storage space 132 under the seat of the bench that can be used to store supplies, equipment, etc. for the medical kiosk. A liftable seat section 134 can be used to access the storage space. When the bench includes a storage space, the bench can include a door 136 which may or may include a lock to limit access to the storage space; however, this is not required. As can be appreciated, the medical kiosk can include one or more chairs, not shown, to enable one or more users to sit in the medical kiosk while receiving medical services in the medical kiosk. The bench is generally about 10-25 inches high, and typically about 16-20 inches high; however, other heights can be used.

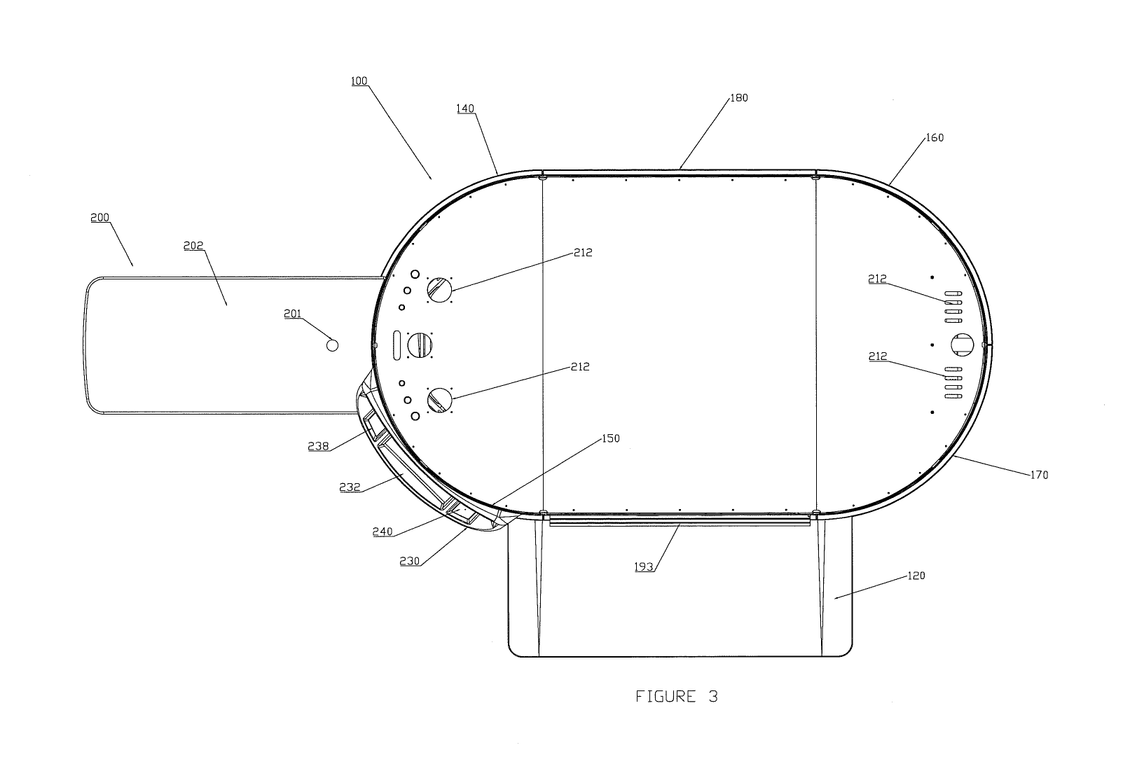

The medical kiosk is illustrated as having two front panels 140, 150, two rear panels 160, 170, one side wall 180, and one door system 190. The front panels, rear panels, side wall, and door system are generally formed of a durable material (e.g., plastic, metal, wood, composite material, man-made materials, etc.). The front panels, rear panels, side wall can have upper trim 142, 152, 162, 172, 182; however, this is not required. As can be appreciated, the medical kiosk can be designed to only include a single front panel and/or a single rear panel. As can also be appreciated, the medical kiosk can be designed to include more than two front panels and/or more than two rear panels. As can be appreciated, the medical kiosk can be designed to include more than one side wall and/or more than one door system. As can also be appreciated, a side wall can be substituted for another door system; however, this is not required. The general shape and size of the front and rear panels are the same; however, this is not required. As illustrated in FIG. 1, the shape of the front and rear panels is arcuate. The radius of curvature is about 10-100 inches, typically 15-50 inches, and more typically about 20-35 inches; however, other radius of curvatures can be used. As illustrated in FIGS. 1 and 3, the front and rear panels have an angle of curvature of about 90 or a quarter of a circle; however, it can be appreciated that one or both rear and/or front panels can have different angles of curvature. The general shape and size of the side wall and the door system are generally the same; however, this is not required. As illustrated in FIGS. 1 and 3, the side wall and door system lie in a generally flat plane; however, this is not required. As illustrated in FIG. 3, the assembly of the front and rear panels and the side wall and door system forms a generally oval shape for the medical kiosk. The two front panels and two rear panels are illustrated as having the same or similar footprint; however, this is not required. Likewise, the side wall and the door system have the same or similar footprint; however, this is not required. The similarity in the shape and footprint of the wall components of the medical kiosk enables the medical kiosk to be assembled in a manner that is convenient for the facility that will include the medical kiosk. For example, if the door system needs to be positioned on the left side of the medical kiosk, instead of the right side, the similarly shaped side wall and door system enables the medical kiosk to be assembled in such a manner. Also, if the registration station of the medical system needs to be placed on the left side or right side or on the rear end of the medical kiosk instead of the front end, the similarly shaped front and rear panels can be easily exchanged to create such configuration for the medical kiosk. The modular medical kiosk not only accommodates multiple configurations of the medical kiosk, it also facilitates in enabling the medical kiosk to be moved into an existing facility and then assembling the medical kiosk in such facility without having to modifying the entry ways into or out of the facility. The thickness and height of the front panels, rear panels, side wall and door system are non-limiting. Generally, the maximum height of the front panels, rear panels, side wall and door system is about 5-12 ft., typically about 6-9 ft., and more typically about 7-8 ft.; however, other heights can be used. The thickness of the front panels, rear panels, side wall and door system is generally about 0.5-10 inches, typically about 1-5 inches, and more typically about 1-2 inches; however, other thicknesses can be used. The front panels, rear panels, side wall and door system can optionally include insulation, sound dampening material, etc.; however, this is not required. The front panels, rear panels, side wall and door system can be designed to be connected together in a variety of ways (e.g., bolted/screwed together, latched together, snap fitted together, press fitted together, etc.). Generally, the arrangement is used to connect together the front panels, rear panels, side wall and door system is selected to enable easy connecting and disconnecting of the front panels, rear panels, side wall and door system from one another. One or more of the front panels, rear panels, side wall and door system can include openings, windows, transparent/semi-transparent regions that allow for ventilation, illumination, and/or viewing; however, this is not required. Generally, front panels, rear panels, side wall and door system are mostly or fully formed of opaque or non-transparent materials so as to ensure the privacy of the user in the medical kiosk; however, this is not required.

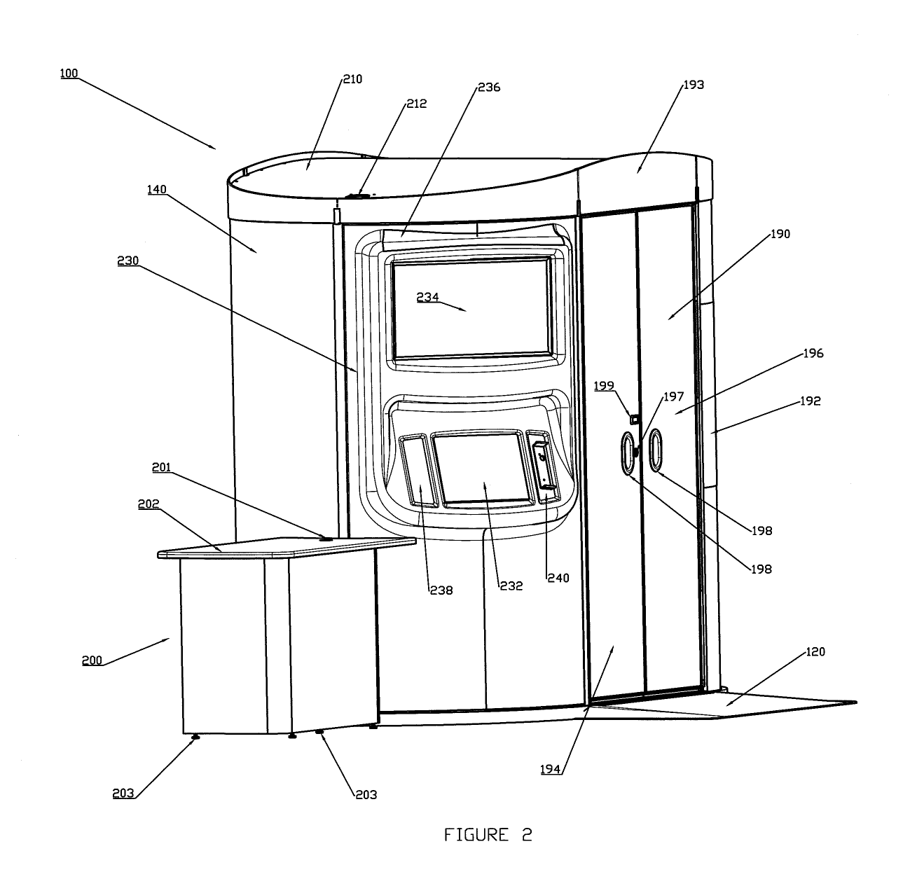

The configuration of the door system 190 is non-limiting. As illustrated in FIGS. 1 and 2, the door system includes a frame 192, 193 and two doors 194, 196; however, it can be appreciated that the door system only includes a single door. One or more both doors can include a handle or grasp cavity 198 on one or both sides of the one or both doors. The one or more doors can be designed to open and close in a variety of ways (e.g., swing open and closed, slide open and closed on a top/bottom rail system, etc.). As can be appreciated, the one or more doors for the medical kiosk can also or alternatively be positioned on one or more of the front or rear panels; however, this is not required. The maximum height of the doors is generally about 5-9 ft., and typically about 6-7 ft.; however, other heights can be used. The maximum width of the entry provided by the one or more doors when fully open is generally about 15-60 inches, typically about 25-55 inches, and more typically about 30-50 inches; however, other widths can be used. The door opening is generally selected to enable a standard wheelchair to pass through the opening; however, this is not required. The door system can optionally include an indicator 199 (e.g., light, message, etc.) that indicates when the medical kiosk is in use or is available. As can be appreciated, the use indicator can be located on other or additional locations on the medical kiosk. The door system can optionally include a lock arrangement 197. The configuration of the lock arrangement is non-limiting. The lock arrangement, when used, can be designed to enable the user to lock the doors to the kiosk medical to prevent access to the interior of the medical kiosk while the user is in the medical kiosk; however, this is not required. The lock arrangement can also or alternatively be used to lock and prevent access to the interior of the medical kiosk when the medical kiosk is not in use; however, this is not required.

The medical kiosk can optionally include an exterior attendant station that is connected to and/or positioned near the medical kiosk. The exterior attendant station can be used by one or more attendants, medical providers, etc. As illustrated in FIGS. 1-5 and 7-9, a desk 200 can be connected to and/or positioned next to an exterior wall of the medical kiosk. The desk can be formed of one or more pieces. When the desk is designed to be connected to an exterior wall of the medical kiosk, such connection arrangement is not limited (e.g., screw, bolt, clamp, press fit, snap arrangement, etc.). The desk can include a desk top 202, one or more legs 203, one or more shelf regions 204, one or more cabinet doors 206, one or more shelves 208, drawers, etc. The one or more doors, when used, can optionally include a lock 207. The desk top 202 can optionally include an opening 201 for cables, etc. One or more chairs, not shown, can be used to allow one or more attendants, medical providers, etc. to sit at the desk. The desk can be used to support a computer, printer, files, supplies, medical devices, refrigerator, medicine, patient blood and/or urine samples, phone, monitor, scanner, credit card reader, etc. The one or more one or more attendants, medical providers, etc. located at the desk can assist a user in using the medical kiosk, maintain and/or clean the medical kiosk, provide medical services to a user of the medical kiosk, etc. The desk is illustrated as positioned at or adjacent to one or both front panels; however, this is not required. Generally the attendant, when used, is not a medical provider; however, this is not required. The attendant, when used, is generally trained to assist a user to use the medical kiosk; however, this is not required. The attendant can 1) provide assistance to a user during the registration process and/or payment process, 2) provide assistance to a user about the medical kiosk and/or how to use the medical kiosk, 3) provide assistance to a user regarding technology in the medical kiosk and/or how to use such technology in the medical kiosk, 4) provide assistance to a user to enter and/or exit the medical kiosk, 5) clean and/or sanitize the medical kiosk, 6) answer and/or assist the user in other ways regarding the medical kiosk and/or services provided by the medical kiosk, 7) assist in the registration, appointment and/or check-in process for a user, etc.

The medical kiosk can optionally include a ceiling panel 210. The ceiling panel can be formed of one or more pieces. The ceiling panel can be formed of a transparent or semi-transparent material to allow light to enter and illuminate the interior of the medical kiosk for partial or fully ambient lighting of the medical kiosk; however, this is not required. One or more lights, not shown, can be positioned on the ceiling panel to illuminate the interior of the medical kiosk; however, this is not required. The ceiling panel is illustrated as including a plurality of vents 212 to enable air to circulate inside the medical kiosk; however, this is not required. One or more fans can be positioned on or adjacent to one or more of the vents; however, this is not required. As can be appreciated, the location of the one or more fans, when used, is non-limiting. As can also be appreciated, one or more fan systems, when used, can also or alternatively be positioned on other components of the medical kiosk (e.g., front panel, back panel, side wall, door system, floor panel, etc.). Generally, the rear vents 212 are designed to allow air into the medical kiosk and front vents are designed to allow air to exit the medical kiosk; however, this is not required. One or more fan systems, when used, and/or vents can optionally include a filter system, to partially or fully filter dust, mites, airborne particles, microorganisms, viruses, etc. from the air prior to the air entering into the medical kiosk and/or prior to the air exiting the medical kiosk; however, this is not required. The filter can include many different arrangements (e.g., HEPA filter, electronic filter, liquid filter, etc.).

The medical kiosk can optionally include a cleaning system that is designed to clean one or more portions of the interior of the medical kiosk and/or kill/neutralize some or all germs and/or other microorganisms in the medical kiosk. One non-limiting cleaning system that can be used is a UV sanitizing system 220. As can also be appreciated, a mist sanitizer can also or alternatively be used to fully or partially clean/sanitize one or more portions of the medical kiosk. As illustrated in FIGS. 1, 8, 9 and 12, the UV sanitizing system 220 can be connected to or positioned adjacent to the ceiling panel and rear panels; however, this is not required. The UV sanitizing system generally includes one or more UV lights that are designed to kill some or all of the germs and/or other microorganisms in the medical kiosk. Generally the germs and/or other microorganisms in the medical kiosk are treated when the interior of the medical kiosk does not include a user. The UV sanitizing system can optionally include one or more standard lights that can be used to provide illumination in the medical kiosk; however, this is not required. The UV sanitizing system can optionally include one or more vents that allow air drawn into the medical kiosk by a fan system to flow into the interior of the medical kiosk; however, this is not required. The UV sanitizing system can optionally include a cooling fan for the one or more UV lights and/or optional standard lights; however, this is not required. The UV sanitizing system can optionally include all or a portion of a mist sanitizing system; however, this is not required. The UV sanitizing system can house one or more cameras, speakers, sensors (e.g., temperature sensor, motion sensor, sound sensor, etc.), etc. for use in the medical kiosk; however, this is not required. The UV sanitizing system includes a shroud 222 that includes vent/light openings 224 to house the components of the UV sanitizing system; however, this is not required. The shape, size and configuration of the shroud are non-limiting. When a mist sanitizing system is additionally or alternatively used, one or more mist nozzles can be located in one or more regions of the medical kiosk so as to direct the sanitizing mist to desired locations in the medical kiosk. The sanitizing system, when used, can be activated by an attendant and/or a medical provider while the medical kiosk is not being used by a user. The doors to the medical kiosk can be closed and/or locked to prevent a user from entering the medical kiosk during a sanitizing process; however, this is not required. The one or more cleaning/sanitizing systems can be used to clean the air in the medical kiosk, clean one or more surfaces of the medical kiosk (e.g., floor, bench, display screen, cabinet surfaces, chair, etc.) and/or a portion of all of the surfaces of a medical device. When the mist system is used, the mist system can be designed to direct the cleaning mist onto one or more locations in the kiosk. When the UV sanitizing system is used, the UV light can be designed to be directed onto one or more regions in the kiosk.

Referring now to FIGS. 1-5 and 7-10, front panel 150 includes a registration station 230. The registration station is illustrated as including a touch screen 232, a display screen 234, and an optional frame 236 that such components can be mounted thereto. The shape of the frame, when used, is non-limiting. The frame, when used, can be designed to be easily removed from the front panel to enable servicing, repair, replacement, etc. of one or more components of the registration station; however, this is not required. As can be appreciated, the registration station can also or alternatively include other or optional features (e.g., additional display screen, additional touch screen, lights, buttons, switches, camera, speakers, microphone, keyboard, scanner, receiver, transmitter, credit card/debit card or other some other card reader, smart phone or other smart device reader/scanner, finger and/or eye scanner, shelf, printer, storage cavity, service access door, motion sensor, sound sensor, temperature sensor, logos, etc.). The touch screen is generally used to allow a user to enter in information about the user (e.g., age, sex, contact information, payment information, medical history, medical issue, etc.). The touch screen can be substituted for a keyboard; however, this not required. The frame is designed to mount the touch screen at some angle (e.g., 10-80°) relative to the front plane of the front panel 150 as illustrated in FIGS. 1, 2 and 5; however, this is not required. The frame optionally includes one or more side sections 238, 240 that can include one or more other or optional features of the registration station. As can be appreciated, one or more other or optional features of the registration station can also or alternatively be located on other regions of the registration station. The touch screen can display various types of information (e.g., electronic keyboard, instructions on how to register, questions that are displayed during registration, instructions during registration, information displayed to user during registration, various templates, various menus, various lists of information, etc.). As can be appreciated, the medical kiosk can be designed to accept voice commands during the registration process; however, this is not required. The display screen can be used to provide various types of information (e.g., registration information, information input by the user, advertising information, information about the medical kiosk, information about wait time for a medical kiosk, information as to the order of users waiting to use the medical kiosk, information about whether a medical kiosk is available or in use, cable TV, satellite TV, local broadcast TV, infomercial, medical programs, DVD materials, Blu-ray materials, video streaming, etc.). Generally, a user enters payment information at the registration station (e.g., swipes a credit or debit card, etc.); however, it can be appreciated that payment information can also or alternatively be entered inside the medical kiosk, at the optional attendant station, wirelessly or over a network via a smart phone or other device or by a computer connected to a network, etc. If an attendant is available, the attendant can assist a user during the registration process; however, this is not required. Generally, the medical kiosk includes a single registration station; however, this is not required. As can be appreciated, the registration station can alternatively be located inside the medical kiosk, at the attendant station, on other panels or sidewalls of the medical kiosk, or located remotely from the medical kiosk (e.g., central registration center for use with multiple medical kiosks, etc.).

Referring now to FIGS. 1 and 8-12, a non-limiting interior of the medical kiosk is illustrated. As previously discussed, the interior room or cavity of the medical kiosk can optionally include a scale 112, a bench 130 and/or a UV sanitizing system 220.

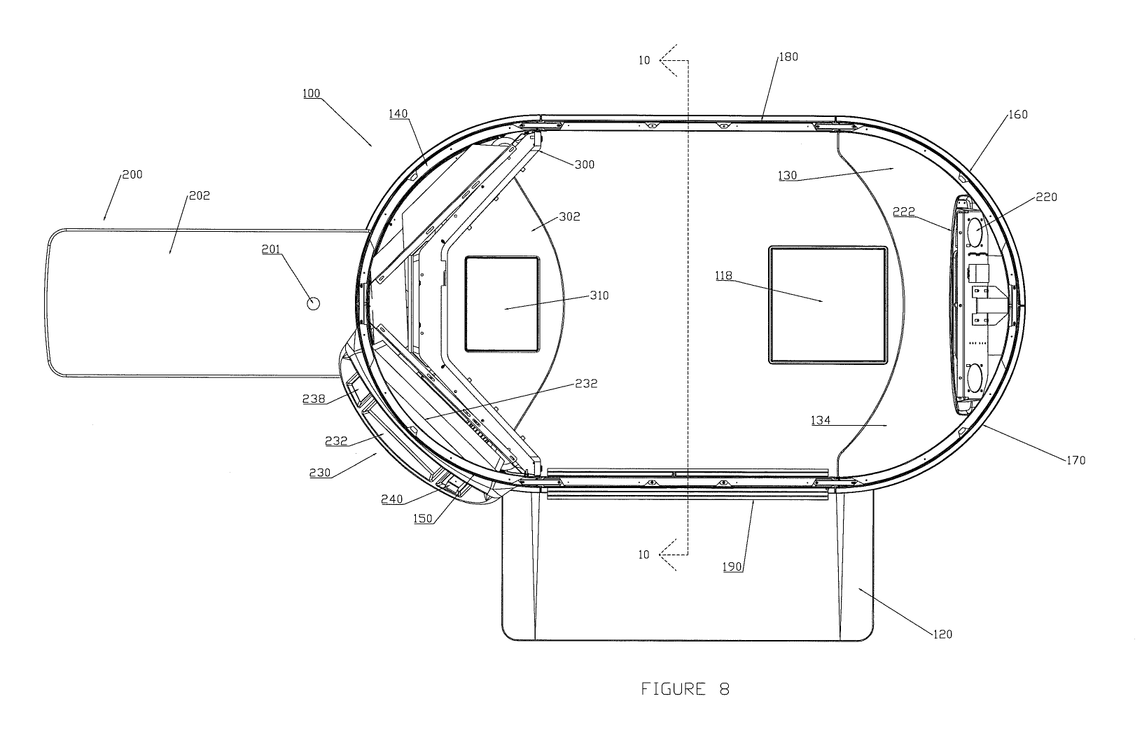

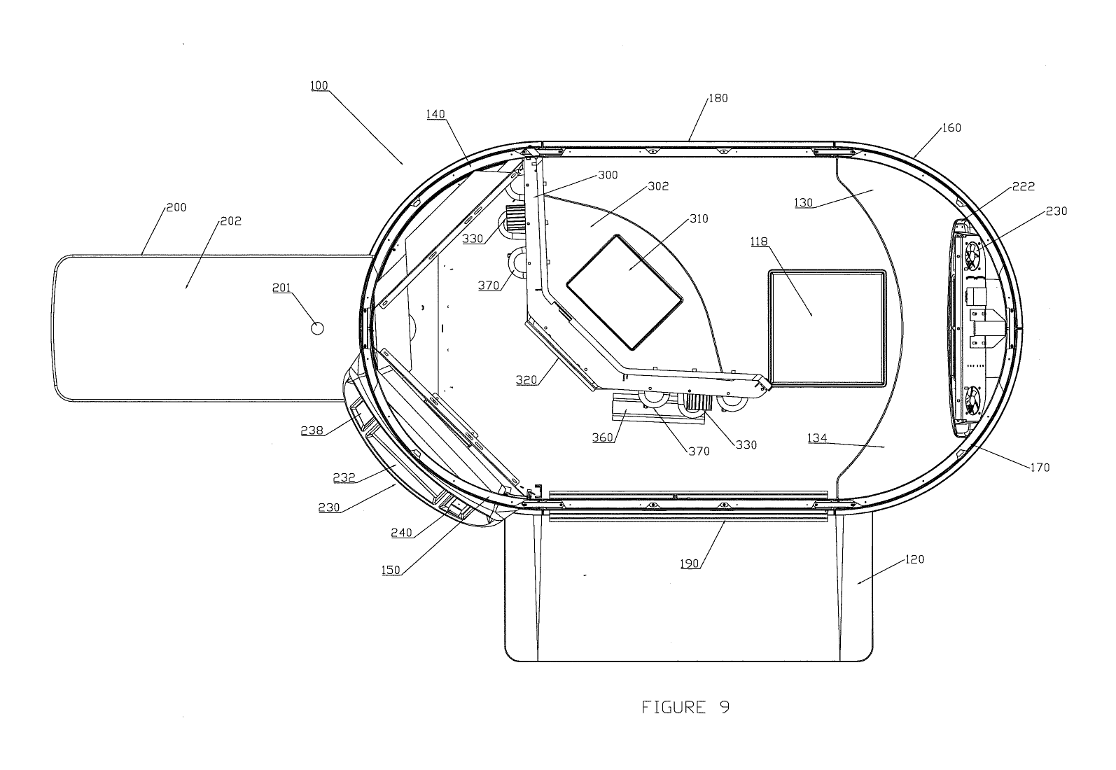

Referring now to FIGS. 8-11, a non-limiting interior front region of the medical kiosk is illustrated. The interior chamber of the medical kiosk includes a front interior panel 300. The front interior panel 300 can be connected to front panels 140 and/or 150. As illustrated in FIG. 9, the front interior panel can be pivotally connected to front panel 140 to enable the front interior panel to be moved away from front panel 150; however, this is not required. The movement of the front interior panel can be used to allow access to the components (e.g. computer, router, server, battery backup, hard drive, medical devices, electronic locks, fans, displays, speakers, camera, headphone jack, electronic scale, Bluetooth devices, lights, pumps, scanners, touch pad, ID verification devices, printer, etc.) that are located between front panels 140 and 150 and front interior panel 300; however, this is not required. Such access can be used to facilitate in the service, maintenance, upgrading, replacement, etc. of such components; however, this is not required. The front interior panel can include a lock; however, this is not required.

The front of the front interior panel can include a desk top 302 that is used to support one or more touch screens 310, touch pads or keyboards positioned on the desk top. The shape, thickness and size of the desk top are non-limiting. The desk top is illustrated as secured to or formed on the front interior panel 300; however, this is not required. The desk top can have one or more support legs, not shown; however, this is not required. The one or more touch screens, touch pads, keyboards, etc. on the desk top can be secured to the desk top; however, this is not required. The desk top is illustrated as sloping downward toward the front edge; however, this is not required. The size, shape and thickness of the one or more touch screens, touch pads, keyboards, etc. are non-limiting. The one or more touch screens, touch pads, keyboards, etc. are designed to be used by a user to enter various types of information (e.g., quality survey, patient history, patient medical information, payment information, questions to medical provider, vitals, etc.) before, during and/or after receiving medical services. As can be appreciated, the one or more touch screens, when used, can additionally or alternatively be used to provide information and/or instructions to the user and/or can provide other uses for the user (e.g., use policy, instructions on how to use the medical kiosk and/or medical devices, providing information on next step of medical visit, request assistance, increase/decrease speaker volume, increase/decrease headjack volume, focus medical devices, adjust volume of medical devices, adjust lighting in kiosk, etc.) during use of the medical kiosk; however, this is not required. The desk top can optionally include one or more other devices (lights, buttons, switches, camera, speakers, microphone, scanner, receiver, transmitter, credit card/debit card or other some other card reader, smart phone or other smart device reader/scanner, finger and/or eye scanner, shelf, printer, storage cavity, motion sensor, sound sensor, temperature sensor, logos, scanner, etc.); however, this is not required. A chair, not shown, can be provided to enable the user to sit when using the medical kiosk. The chair can be a free standing chair or be connected to the medical kiosk.

One or more monitors or display screens 320 can be positioned on the front of the interior front panel. As can be appreciated, one or more monitors or display screens can be positioned on other or additional locations in the medical kiosk. The shape, size and thickness of the one or more monitors are non-limiting. The monitor is generally used to view the one or more medical providers when the user is located in the medical kiosk. The one or more monitors can also or alternatively display other or additional information (e.g., instructions, questions, general medical information, time, output or results of examination of user, vitals information, results from the medical devices, advertisements, information about the medical kiosk, information being displayed and/or entered by the user on the touchscreen, etc.).

One or more cameras (e.g., video camera, etc.) can be positioned on the interior wall panel and/or be embedded in the one or more monitors or display screens 320. As can be appreciated, one or more cameras can be positioned on other or additional locations in the medical kiosk. The one or more cameras enable pictures of the user in the medical kiosk to be transmitted to a remotely located medical provider. The remotely located medical provider also typically includes a camera at his/her location so that pictures of the medical provider can be transmitted to the one or more monitors or display screens 320 in the medical kiosk. Such an arrangement can allow for real-time or near real-time video conferencing between the user and medical provider while the user is located in the medical kiosk. The one or more cameras can have other or additional functions (e.g., determine height of user when standing on scale or other locations in the medical kiosk, view one or more regions of user so as to provide a medical examination of the user in the medical kiosk, monitor occupancy of the medical kiosk, provide security monitoring for the medical kiosk, etc.). The one or more monitors or display screens 320 in the medical kiosk can also be used to replay one or more portions of the video conference between the user and medical provider after the secession with the medical provider has ended; however, this is not required. Such a feature, when available, enables the user to review or again listen to instruction, advice, etc. provided by the medical provider to the user. This video playback feature, when available, can be limited to being viewed by the user while the user remains in the medical kiosk, or can also or alternatively be accessed be the user after the user exits the medical kiosk; however, this is not required. If the user can optionally access the recorded video conference outside the medical kiosk, the video file can 1) be accessed from some central server, 2) electronically sent to a personal computer, mobile device, tablet, laptop, etc., and/or 3) mailed to the user (e.g., DVD, Blu-ray disk, video tape, USB jump drive, etc.).

One or more speakers 330 can be positioned on the front interior panel of the medical kiosk. As can be appreciated, one or more speakers can be positioned on other or additional locations in the medical kiosk. The speakers can be used to enable a user in the medical kiosk to listen to what the medical provider is saying to the user. One or more microphones are generally included in the medical kiosk to enable the user to verbally communicate with the medical provider. The medical kiosk can optionally include a braille keyboard and/or reader to enable the visually and/or hearing impaired to communicate with a medical provider while in the medical kiosk. The speakers can also or alternatively be used to play background music, sound an alarm, etc. A headphone jack 340 can be provided on the interior front panel and/or other locations in the medical kiosk to enable headphones to be connected to the medical kiosk; however, this is not required. The interior front panel and/or other locations in the medical kiosk can include one or more data ports (e.g., USB, Ethernet, Firewire, etc.) to enable medical devices, storage devices, smart phones, computers, tablets, and/or other devices to be connected to the medical kiosk; however, this is not required. For example, the data port could be used by a patient, to download and/or upload information to a medical device on a patient (e.g., heart monitor, heart pacemaker, implantable cardioverter defibrillators, etc.).

The front interior panel 300 can optionally include one or more air vents 350. The one or more air vents, when used, can enable air to enter or exit an interior location positioned between the front wall panels and the front interior panel to thereby provide circulation in the medical kiosk. Such interior location can include one or more fans to draw air through the one or more vent and into the interior location; however, this is not required. As illustrated in FIGS. 8 and 8, the interior location can include a computer 360, a router, a server, a battery backup, a hard drive, medical devices, electronic locks, displays, speakers, camera, headphone jack, Bluetooth devices, lights, pumps, devices, printer, etc. Such devices can generate heat during operation, thus the air flow can be used to provide cooling for such devices.

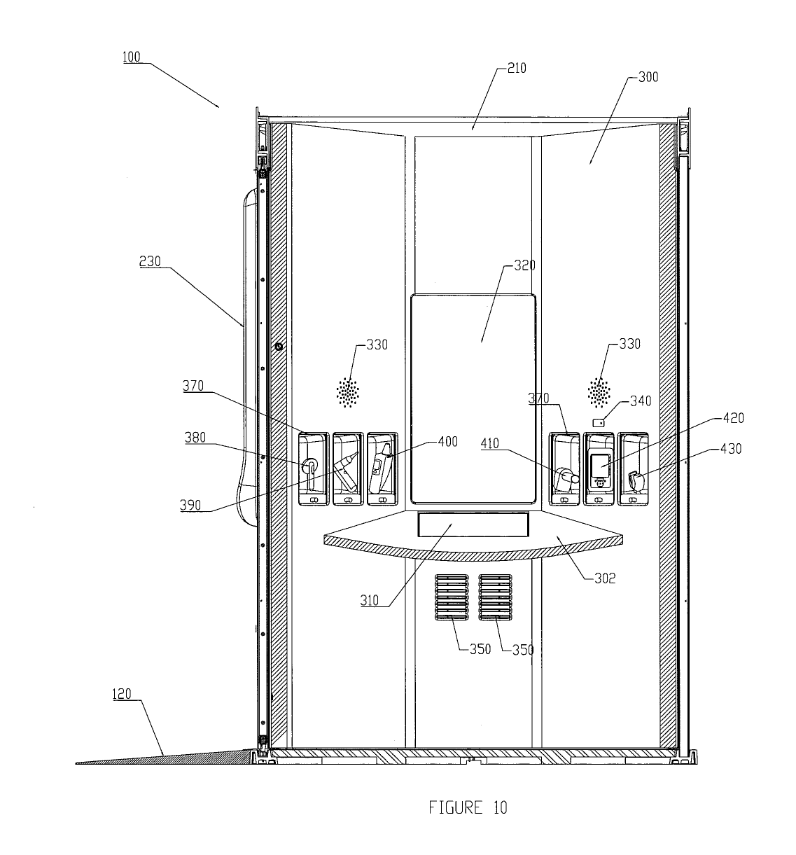

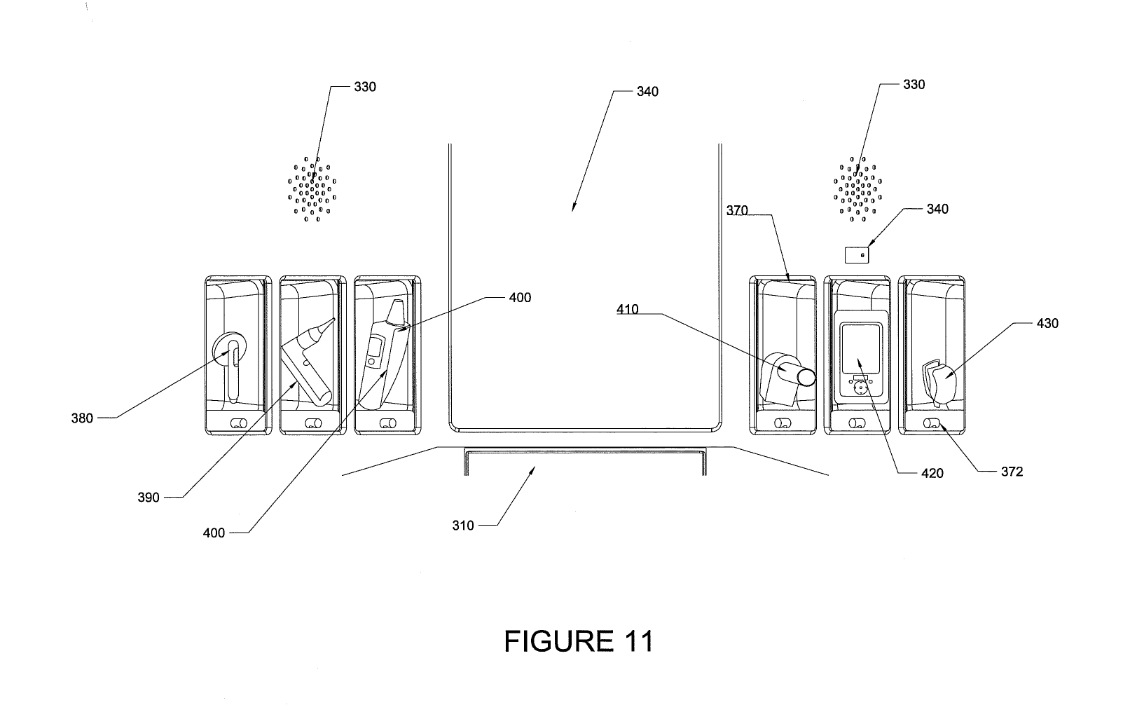

The front interior panel can include one or more equipment chambers 370 can be positioned on or near the front interior wall. The equipment chambers are used to store one or more medical devices (e.g., stethoscope, otoscope, thermometer, dermascope, spirometer, pulse oximeter, heating pad, magnifying glass, tongue depressor, tweezers, blood glucometer etc.). The one or more equipment chambers can also or alternatively be used to include other types of materials (e.g., tissue, Band-Aid, gauze, cotton ball, disinfecting wipe, cortisone cream, antibiotic cream/ointment, cotton swab, fabric wrap, etc.). The one or more equipment chambers generally include a door 372 to limit access to the one or more equipment chambers; however, this is not required. The door, when used, can be manually openable/closeable, and/or the doors can be controllably opened/closed remotely by the medical provider and/or attendant. Generally, the doors are controllably opened and/or unlocked by the medical provider during the examination of the user in the medical kiosk. After the user has left the medical kiosk, the attendant can enter the medical kiosk, and then clean the medical equipment that was handled or used by the prior user and/or dispose of and/or replace items that were used and/or handled by the prior user. Thereafter, the attendant can restock, replace, and/or reposition the medical equipment and/or non-medical equipment in the equipment chambers and close the equipment chamber doors prior to the next user entering the medical kiosk. One or more types of medical equipment can be designed to transmit information by wire or wirelessly to electronic components in the medical kiosk and/or to the remotely located medical provider. As illustrated in FIGS. 10 and 11, six equipment chambers having doors are included in the medical kiosk, three on each side of the desk top 300. As can be appreciated, a larger or smaller number of equipment chambers can be used. As also can be appreciated, some or all of the equipment chambers can be absent doors. The doors on the six equipment chambers are designed to be unlocked and/or opened remotely by the medical provider and/or during the taking of vitals by the patient. The doors are designed to automatically lock closed when the doors are closed by the attendant after the user has left the medical kiosk; however, this is not required. Each of the six equipment chambers is designed to include a different piece of medical equipment, namely a stethoscope 380, an otoscope 390, a thermometer 400, a dermascope 410, a spirometer 420, and a pulse oximeter 430. As can be appreciated, a larger or smaller number of medical equipment can be used in the medical kiosk and/or different types of medical equipment can be included in the medical kiosk.

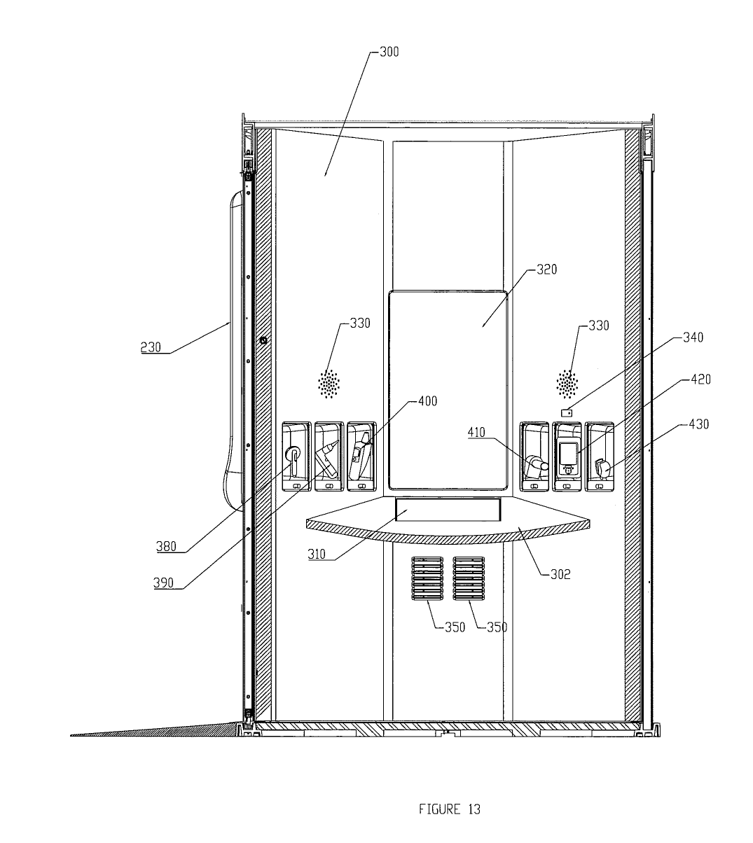

Referring now to FIG. 13, there is illustrated an alternative configuration for the medical kiosk 100 of the present invention. In this embodiment, floor 110, doors 190, panels 140, 150, 160 and 170, ceiling 210, ramp 120, bench 130 and UV sanitizer 220 are absent from the medical kiosk. The attendant desk 200 may also be absent. In this non-limiting arrangement, the medical kiosk 100 can be placed in a private room and/or some other location. The medical kiosk includes a registration station 230 that can be the same or similar as the registration station described above. The medical kiosk can include front wall panels 140 and 150 or a single wall front wall panel. The medical kiosk includes interior front panel 300 which can be the same or similar to interior front panel 300 as described above in FIGS. 1 and 8-11. The interior front panel can be pivotally connected to the one or more front wall panels; however, this is not required. The interior front panel may or may not include a Physician Screen 340. If interior front panel is the same or similar to interior front panel 300 as described above in FIGS. 1 and 8-11, then the user can have a teleconference with a medical provider. However, if interior front panel does include a Physician Screen 340 and one or more other components (e.g., speakers 330, headphone jack 340, etc.), then the user may only be able to collect one or more vitals and then proceed to another location to visit a medical provider. As can also be appreciated, the medical kiosk can also be absent the desk top, medical device compartments, Patient Screen and the like such that the medical kiosk primarily includes the registration station 230 to enable a user to check-in for a visit with a medical provider. As can be appreciated, one or more features of the medical kiosk of the present invention can be used in other ways to provide medical services to a user.

The medical kiosk and method for using the medical kiosk are a novel and advanced healthcare delivery system wherein patients and physicians can engage in real-time interactive consultations, providing convenient and affordable healthcare services. The medical kiosk includes the latest technologies in medical devices, video conferencing, and VOIP telephony so that the medical kiosk can extend traditional healthcare to convenient retail pharmacy locations or other locations in a user's neighborhood, therein enabling a user to see a medical provider and obtain a prescription, if required, in a fast and convenient manner.

Some advantageous aspect of the medical kiosk and medical method are:
Patient Portal (Cloud Based).
Provider Portal (Cloud Based).
Integrated Care Station.
Facilitates Efficient Delivery of Basic Healthcare Delivery.
Automates All Aspects of a Check Up.
Easy Check-In.
Vital Signs Capture.
Prescription Generation.
Post Care and Outcomes.
Convenient Locations Where Consumers Want To Be.
Video playback of the recorded session between the user and medical provider.

The medical kiosk and medical method can be used to provide primary and/or urgent care services in four (4) simple steps:

Step 1—Patient Begins/Completes Check-in Process Via Web Portal or at the Medical Kiosk.

The patient can optionally begin the check-in via web portal and then later complete check-in process at the medical kiosk; however, this is not required. A medical provider can send a reminder to patient regarding an appointment and/or begin the check-in process for a patient (e.g., follow-up appointment, etc.); however, this is not required.

Step 2—Medical provider receives eligible request and accepts and/or is assigned to patient.

Step 3—Patient visits the medical kiosk and has a private appointment with a doctor via the Patient Screen.

Step 4—Visit is completed. The medical kiosk and/or medical provider can then provide additional care/services that include: prescription, billing information, education, referrals, follow up and/or EMR/PHR entry. The medical provider can cause the medical kiosk to printout a prescription and/or directly send the prescription request to a pharmacy. The medical kiosk can print out a bill after the medical services are provided and/or accept payment prior to or after medical services are provided. The medical kiosk can be designed to accept and/or process medical insurance information provided by the user. The medical kiosk can print out and/or display education materials/information relevant to/requested by the user and/or provided by the medical provider. The medical kiosk and/or medical provider and/or attendant can schedule a follow-up visit for the user. Email, twitter, Facebook, test, and/or mail reminders can be sent to the user regarding scheduled and/or follow-up visits. The medical provider and/or attendant can schedule a visit with another medical provider and/or admit the user to the hospital, contact an ambulance, etc. during or after the visit to the medical kiosk. A visit summary can be printed out and/or sent to the patient. As can be appreciated, the medical kiosk and method for using the medical kiosk can have other or additional features.

Advantageous portal features of the medical kiosk and associated medical method are:
Practice Management Engine.
   Appointments Scheduling Engine.
   Online Eligibility, Claims, and Billing Engine.
   ePrescribing with Alerts and Reminder Engine.
   Medical Records Interface and Access.
      Personal Health Record (PHR).
      Electronic Medical record (EMR).
      Rules-Based Care Plans.
      Rules-Based Education.
Check In Pathway to Care Engine.
Secure Video Conferencing Engine.
Documentation Module.
   Appointment Storage and Analysis.
Education and Post Care.

Some non-limiting advantages to patients by use of the medical kiosk and medical method are:
Convenient.
   Closer to home.
   Saves time.
   Language and culture friendly.
Better Access.
   Personal doctor available while traveling.
   Larger selection of doctors.
   Not limited by doctor's visitation schedule.
More Accurate.
   Review record of appointment.
   Automatic data entry into PHR.
Less Exposure to Illness.

Some non-limiting advantages to medical providers by use of the medical kiosk and medical method are:
Higher Revenues.
   More appointments/day.
   Less traveling.
More Accurate.
   Review record of appointment.
   Automatic data entry into EMR/HER.
Integrated Care.
   Referral and transfer.
Load Balancing.

Appointment load can be shared with other doctors regardless of location.

Some non-limiting advantages to payers by use of the medical kiosk and medical method are:
Change in Status.
   Transition from Payer to Provider.
Market Leverage.
New Business Model.
Call Center based Nurse Practitioner.
Efficiency.
   Market Demand.
   Less Overhead.
   Scalability.
   Less Liability.

The medical kiosk of the present invention is an Integrated Care Terminal that is a highly equipped doctor's office that is built and designed to deliver urgent and minor medical care in the field utilizing a centralized team of doctors for the evaluation and treatment of patients. The medical kiosk can be fitted with the latest FDA approved medical devices used by doctors today.

Employing the latest technology that is used in physician offices and emergency rooms, the medical kiosk is able to allow patients to obtain appropriate care in locations that are convenient, accessible, and more affordable.

The medical kiosk is designed to be a comfortable, self-contained, secure, sanitary, soundproof kiosk that is approximately 5 feet wide and 9½ feet long; however, it can have other dimensions. The medical kiosk can be made of extruded plastics and related components. The interior of the medical kiosk can contain one or more of the following integrated medical devices:
Thermometer (e.g., temperature taken via ear, temperature taken via ear, IR thermometer to scan head or other area of body, etc.).
Scale built into the patient seat or floor for measuring the patient's weight.
Otoscope—for examining the middle ear, exterior ear, nasal passages, mouth and throat.
Oximeter which measures the blood oxygen saturation.
Stethoscope for evaluation of heart, lung and bowel sounds.
Blood Pressure Cuff to measure blood pressure.
EKG which provides a snapshot of the heart rhythm and data regarding stress or injury to the heart muscle.
Spirometer and transducer for measuring lung function.
Blood glucose measuring device or monitor.
Retinal scan device (e.g., Itronix retinal scan device, etc.).
Dermascope.

A medical attendant (e.g., medical assistant, nurse assistant, nurse, nurse practitioner, physician, etc.) who resides outside the medical kiosk can be responsible for answering user questions, assisting with user registration/payment, thoroughly clean the medical kiosk after each use, and restock and/or reset the medical kiosk after each use. The cleaning of the medical kiosk can include sanitizing the seat, touch screen, floor, walls, seat, and all instruments as well as ensuring the medical kiosk is free of debris and any patient belongings. The medical kiosk can also be designed to be automatically sterilized after one or more users use the medical kiosk by utilizing a chemical mist sterilization technology and/or UV sterilization technology. The medical attendant can also ensure that any insurance forms required by the user/patient for reimbursement are provided via a printer or some other means contained in the exterior and/or interior of the medical kiosk.

The medical kiosk can also contain one or more computers, which are connected to the internet and powers the one or more monitors and/or other type of equipment in the medical kiosk; however, this is not required. The exterior monitor on the medical kiosk can be used for patient registration and appointment selection that can be conducted in a touch screen format.

The method for providing medical services via a medical kiosk regarding protocols for scheduling, diagnosing, delivering and documenting tele-medicine primary care can include:

a. Medical Provider Application—this application is used by the medical provider to provide clinical services. The application contains all that the medical provider requires to diagnose, deliver care and document the clinical episode. It runs on the physician's computer and can be integrated with the leading EMR applications.

b. Patient Application—this application is used by the patient to register with a medical kiosk and also captures the patient's medical history and/or vitals. It includes all the information required to administer clinical services to the patient. This includes financial/billing information and a Healthspot Electronic Medical Record (EMR), which can be accessed by the patient and the medical providers.

c. Integrated videoconferencing software—this application supports the live patient-clinician interaction required for delivery of the clinical services. It uses a secure connection to the servers and the provider via an internet connection.

To use a medical kiosk, the users/patients may go through one or more of the following steps:

a. Go Online, register and schedule an appointment at the nearest terminal or walk-in and register at the medical kiosk.

b. Use the scheduling engine to select an appointment time.

c. Input insurance, preferred pharmacy location and/or billing information, and remit payment.

d. Complete pre-appointment pathway to care.

e. Visit a medical kiosk and see a medical provider via the integrated care terminal.

f. Pick up prescription, if indicated, at the pharmacy of choice.

g. Optionally use website to manage user's care until user is better.

The following example is a non-limiting example as to what one user may encounter when using the medical kiosk of the present invention:

Jane Doe is not feeling well, suffering from severe nasal congestion and “cold symptoms” for several days. She realizes she should seek care, but finds it frustrating to schedule an appointment with her primary care physician and knows that this is not an appropriate reason to visit her local ER. However, she recently became aware of a medical kiosk in her neighborhood grocery store, and decides to drive to the store a few minutes from her home to see if she can get a walk-in appointment. Upon arrival, Jane is met by the attendant who helps her register at the exterior of the medical kiosk for the next available appointment in a few minutes. She completes the basic information about her medical condition in a short guided questionnaire via the monitor on the exterior of the terminal. The intake questionnaire captures demographic information that can include some or all of the following information: name, address, gender, race, native language, age, birthdate, fingerprint, simple medical history/medications, current symptoms, allergies, medical condition, medications currently used, preferred pharmacy, insurance information, and a debit/credit card payment and/or some other types of payment for the visit, and/or time of next available visit in kiosk. Information is gathered and stored securely to facilitate future visits. Jane then has a seat at a small waiting area near the medical kiosk. When the appointment prior Jane's ends by another user, Jane witnesses the attendant cleaning the appropriate surface areas of the interior of the medical kiosk, and the changing the protective covers of the instruments that were used.

Jane then proceeds to check-in to the kiosk by providing a password or some other type of ID. This check-in can occur with the registration station on the kiosk and/or with the medical attendant. Once checked-in, the medical attendant escorts the patient into the medical kiosk. The medical assistant also fits a blood pressure cuff on the patient, connects the blood pressure cuff to the medical kiosk, and then begins the vital capture process for the patient. The medical attendant may or may not be present during the complete vitals capture process. During the vitals capture process, the weight and temperature of the patient is also collected. The Patient Application provides information to the patient on how to proceed with the vitals capture process. Once the vital capture process is completed, the patient presses a button or indicates in some other manner that the patient is ready to proceed with the teleconference with the medical provider. During the vitals capture process, the medical provider can view Jane's medical history, current medications, symptoms and/or any other information that Jane's entered during the registration process. The medical provider can also view the results of the vitals that were captured during the vitals capture process. Once the medical provider receives information that the patient is ready for the teleconference, the medical provider selects the option to create such connection. The medical provide then appears on the Provider Screen in the medical kiosk and the patient appears on the screen being used by the medical provider. The medical provider can then question and examine Jane during the teleconference.

After the medical provider has spoken to Jane, he suspects a possible sinus infection. The medical provider then clicks on a button on the Provider Application which causes a door to a medical device cavity or compartment on the medical kiosk to open for the otoscope. The medical provider then instructs Jane to gently insert the device in her nose and takes a picture of her nasal mucosa. The picture is displayed on the medical provider's screen and optionally displayed on the Patient and/or Provider screen in the medical kiosk for Jane to see. The medical provider proceeds to instruct Jane to use the instrument to look in her ears and throat. The medical provider views the pictures and determines that the throat and nasal passages are inflamed which is indicative of an upper respiratory infection. Based on her symptoms and the examination, the medical provider tells Jane he believes that she has a sinus infection.

The medical provider can optionally prescribe a prescription for the appropriate antibiotic which can be e-prescribed by the medical provider and sent electronically to the pharmacy of Jane's choice, which in this case happens to be the pharmacy within the grocery store. Prior to or after Jane exits the medical kiosk, Jane may take a survey of the visit. Jane may optionally complete an insurance form from the attendant, which she can use to file with her insurance company for reimbursement. Jane then walks over to the pharmacy and waits on her prescription, which is available ten minutes later. She returns home and begins taking the medication to cure her sinus infection.

Some of the non-limiting features of the medical kiosk are:
Integrated Medical Devices.
Exterior Check-In Station.
Patient Waiting Area.
Integrated Wi-Fi Hot Spot.
Touch Screen User Interface.
HIPAA Compliant Design.
Instant Sterilization.
Video/Audio Conferencing.
Flexible Access.
   Handicap.
   Parent and Child.
Modular design for Pharmacy door deployment.
Small Footprint.
Fully Integrated Interior Design.
Expandable Device Rail.
Secure PC storage with access.
Open design feels comfortable.
Payment and Signature.
Finger Print Reader.
Integrated Printer.
Integrated Medical Devices.
   Thermometer—(e.g., temperature taken via ear, temperature taken via ear, IR thermometer to scan head or other area of body, etc.
   Scale built into the patient seat or floor for measuring the patient's weight.
   Otoscope—for examining the middle ear, exterior ear, nasal passages, mouth and throat.
   Oximeter which measures the blood oxygen saturation.
   Stethoscope for evaluation of heart, lung and bowel sounds.
   Blood Pressure Cuff to measure blood pressure.
   EKG which provides a snapshot of the heart rhythm and data regarding stress or injury to the heart muscle.
   Spirometer and transducer for measuring lung function.
   Blood Glucose measurement device and/or monitor to measure blood glucose levels.
   Retinal scan device to view structures in the eye.
   Dermascope to view the skin and/or throat.
   Blood analyzer to measure different chemicals in the blood (e.g., glucose, serum albumin, creatinine, etc.) and/or other characteristics of the blood (e.g., blood cell count, enzyme levels, ion levels, measure ability of blood to clot, etc.).
Exterior Check-In Station—A monitor and keyboard is generally mounted on the outside of the medical kiosk to allow for new user/patient registration and check in. The station can be designed to take payment and/or a fingerprint. The station generally is located away from the entrance to the medical kiosk to allow a degree of separation from the patient inside the medical kiosk. The attendant can use this station for her work.
Patient Waiting Area—Can include a small area to put a few chairs outside the medical kiosk to act as a waiting area.
Integrated Wi-Fi Hot Spot—In order to minimize network connection cost, a Wi-Fi hot spot can partner with an ISP of choice (AT&T, Verizon, Sprint, T-Mobile, etc.).
Touch Screen User Interface—The patient can be allowed to interact while inside the medical kiosk by use of a touch screen interface.
HIPAA Compliant Design—HIPAA requires patient information to be secure. This will mean the medical kiosk will generally be sound proof and a passerby cannot see in and see any patient information. The medical kiosk generally is fully enclosed and lockable, but can allow exterior access in case of emergency.
Instant Sterilization—Because of the many germs and other contaminants that will be inside the medical kiosk, a sterilization technology can be used in the medical kiosk. One type of sterilization system that can be used is a built-in sanitizing misting system that dispenses from a series of misters between every appointment. Another or additional sterilization system that can be used is a UV lighting system that can be blasted between appointments. The one or more cleaning/sanitizing systems can be used to clean the air in the medical kiosk, clean one or more surfaces of the medical kiosk (e.g., floor, bench, display screen, cabinet surfaces, chair, etc.) and/or a portion of all of the surfaces of a medical device. Other techniques and technologies can also or alternatively be used. The attendant can have the ability to activate one or more sterilization systems (e.g., via button, computer, etc.). The attendant can be required to keep track of records about the sanitization process and can ensure that the doors to the medical kiosk are closed/locked during the sterilization process.
Video/Audio Conferencing—The patient will communicate with the medical provider via video and audio conferencing technology. This environment can make the patient feel as close to the medical provider as actually being present as possible. A two-way glass can be used to place the camera in the center of the monitor to keep the patient looking head on, versus the Skype and current video conferencing solutions that keep users looking at the camera and back to the monitor.
Flexible Access—The medical kiosk should have a large enough door to accommodate a wheel chair. It should also be large enough inside to allow a parent to sit with a child and not feel constrained.
Modular design for Pharmacy door deployment—Most Pharmacies today only have a standard door. The medical kiosk is built in a manner that allows it to enter through the door and quickly be assembled.
Small Footprint—Because of the cost of retail space, the medical kiosk will be small enough to fit in most locations (e.g., 4-6 ft. wide and 7-10 ft. length).
Fully Integrated Interior Design—The interior of the medical kiosk can be designed to be cleaner, sleeker, nicer, more luxurious, than the experience they get at the average doctor's office.
Expandable Device Rail—The medical kiosk can include the latest medical devices and update such medical devices in a more rapid manner than the average doctors' office. The medical kiosk can include a mounting rail type system that allows medical devices in the medical kiosk to be easily accessed by the user. An indicator, such as a light, can be used to notify which medical device is to be used by the user.
Secure PC storage with access—Because uptime of the software is so important, electronics can be inserted in a compartment in the medical kiosk. Such area generally should be secure, cooled, and easily accessible for service.
Open design feels spacious—The medical kiosk is generally designed to feel bigger than it is such as by providing a glass window that wraps around the back half of the kiosk.
Payment and Signature—The medical kiosk can include an integrated credit card swipe for payment, and a signature pad for medical authorization.
Finger Print Reader—A finger print reader can be included on the medical kiosk to confirm patient ID under HIPAA.
Integrated Printer—An integrated printer can be included in the medical kiosk to print medical and insurance forms and/or receipts and/or prescription. The printer can also print coupons based upon diagnosis to promote product sales. Our software can be included to inform the attendant of low paper in the paper.
Video Playback—The recorded medical session can be partially or fully reviewed by the user to enable the user to again listen to information, instructions and/or advise from the medical provider. As can be appreciated, the video playback feature can also or alternatively be used for auditing purposes, compliance purposes, security purposes, quality control purposes, etc.

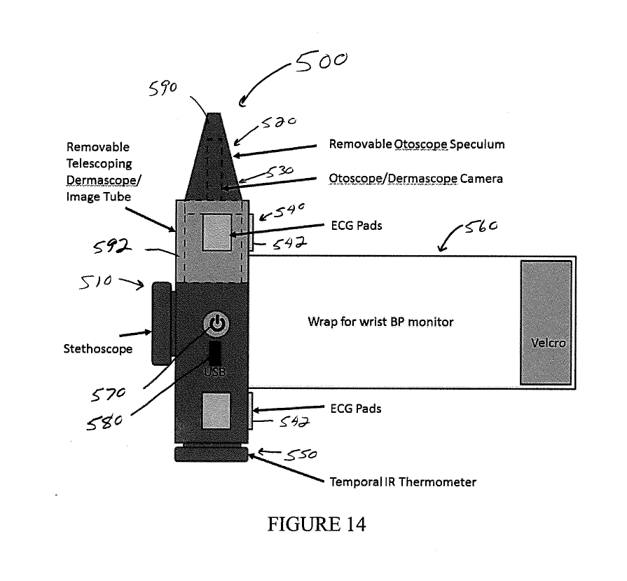

Referring now to FIG. 14, is a side view of a non-limiting embodiment of a multipurpose home and/or mobile medical device 500 that includes a plurality of medical devices. The size, configuration, design and materials of the mobile device are non-limiting. The mobile device includes a plurality of medical devices. The number and type of medical devices incorporated in the mobile device is non-limiting. As illustrated in FIG. 14, the mobile device includes 500 with a plurality of medical devices that can be contained in a common housing. The multipurpose home and/or mobile medical device is designed to be simple to use. The mobile device illustrated in FIG. 14 includes a stethoscope 510, an otoscope 520, a dermascope 530, an ECG 540, a thermometer 550, and a blood pressure cuff 560. As can be appreciated, the mobile device can include other or additional medical devices. The mobile device is generally used with a personal medical kiosk. The mobile device can be connected to the medical kiosk via one or more known interfaces such as, but not limited to, hard wire, USB, HDMI, Firewire, Bluetooth, IR, wireless technologies, etc. The mobile medical device can includes a power button 570; however, this is not required. The mobile device can include a USB connection 580; however, this is not required. The medical device can include a removable cap 590 or body portion 592; however, this is not required. The removable cap is illustrated as covering/protecting the otoscope or some other medical device. The removable body portion is illustrated as protecting and/or providing access to the dermascope or some other medical device. The mobile device is illustrated as including two ECG pads 542. The pads can be positioned on any region of the mobile device. The thermometer is illustrated as an IR thermometer; however, other types of thermometers can be used. The dermascope and otoscope are illustrated as being partially or fully removable from the housing of the mobile device; however, this is not required. The blood pressure cuff can be used to test for blood pressure and/or pulse rate of a patient. The blood pressure cuff is illustrated to be connected at one end to the side of the housing of the mobile device; however, this is not required. The blood pressure cuff is illustrated as including a Velcro connection; however, other or additional connection arrangements can be used. The mobile device can be designed to collect vital information and/or gather images so that a remotely located healthcare provider can be provided with such collected information and make a sound and informed diagnoses of various conditions. The mobile device is designed to be simple enough for a patient to use on themselves in a consultation with a medical provider.

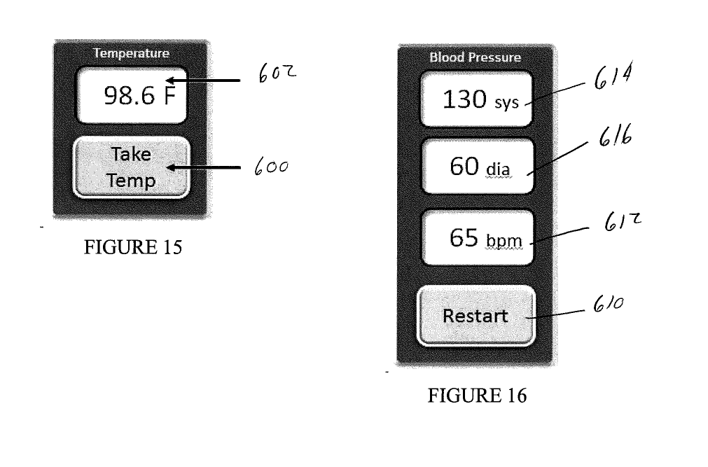

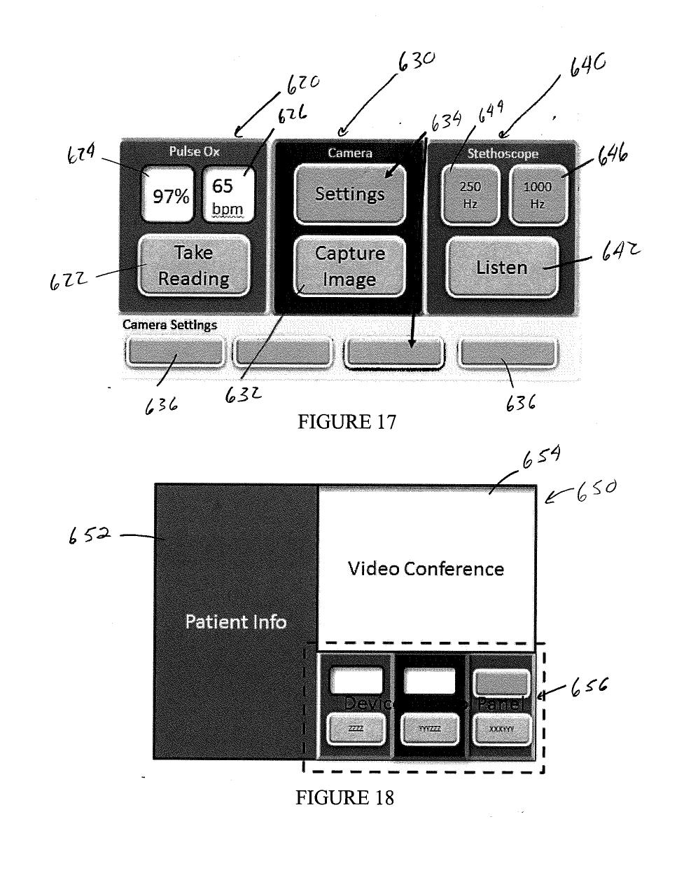

Referring now to FIGS. 15-18, several medical non-limiting device panels are illustrated. The medical kiosk can be designed to include a medical device panel and associated software to aggregate a plurality of medical devices into a single medical device panel and software system. As illustrated in FIG. 15, there is provided a medical device panel that is used to control a thermometer. The panel includes a button 600 to begin a temperature test. The panel also includes a display 602 for indicating the temperature. As can be appreciated, the panel can include additional information and/or functions and/or have a different configuration. As illustrated in FIG. 16, there is provided a medical device panel that is used to control a blood pressure cuff. The panel includes a start/restart button 610 to begin the blood pressure/pulse test. The panel also includes displays for the pulse rate or beats per minute (bpm) 612, the systolic reading (sys) and the diastolic reading (dia) 614, 616. As can be appreciated, the panel can include additional information and/or functions and/or have a different configuration. Referring now to FIG. 17, there is provided a medical device panel that includes controls for a plurality of devices. The device panels are a pulse oximeter 620, an otoscope and/or dermascope 630, and a stethoscope 640. As can be appreciated other or additional devices can be included on the panel. As also can be appreciated, individual panels can be used for each device. As illustrated in FIG. 17, the pulse oximeter panel includes a start button (Take Reading) 622 for the pulse oximeter and a display for oxygen saturation percentage 624 and a display for pulse rate or beats per minute (bpm) 626. The otoscope and/or dermascope panel includes a start or Capture Image button 632 for the camera in the otoscope and/or dermascope. The panel also includes a camera settings button 634 which can be used to activate one or more camera setting buttons (e.g., zoom, focus, color, resolution, etc.) 636. The panel also includes a start or Listen button 642 for the stethoscope. The stethoscope panel can include an amplifier or hertz selection button (e.g., 250 Hz, 1000 Hz, etc.) 644, 646. As can be appreciated, the panels can include additional information and/or functions and/or have a different configuration. Referring now to FIG. 18, there is an example of a medical device panel 650 used to control the tele-medicine medical conference. The panel can be used by the medical provider and/or a patient. As illustrated in FIG. 18, one portion 652 of the panel is used to display or access the patient information. Another portion 654 of the panel is used to activate/deactivate the videoconference between the medical provider and the patient. Another portion 656 of the panel is used to access/activate one or more medical devices that are to be used during the tele-medicine session. As can be appreciated, the panel can include additional information and/or functions and/or have a different configuration.

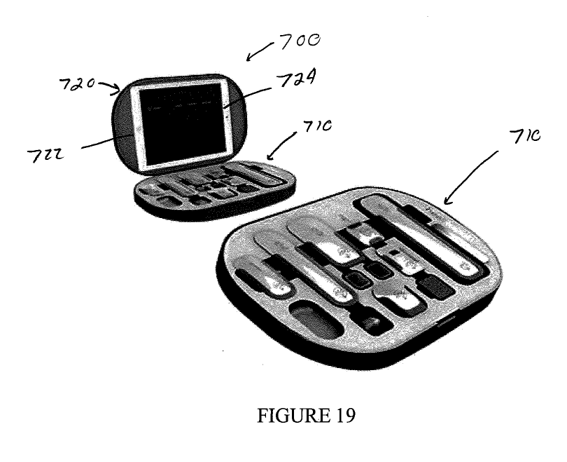

Referring now to FIG. 19, there is illustrated a non-limiting medical kit 700 that includes one or more medical devices that can be used with a personal medical kiosk. The medical kit 700 includes a base 710 which has a plurality of compartments for holding one or more medical devices. The one or more compartments can also contain other materials and/or devices (e.g., band-aid, bandage, gauze, tweezers, scissors, etc.). The medical kit can be made of various types of materials (e.g., plastic, metal, etc.) The shape, size and configuration of the medical kit are non-limiting. The medical kit can be shaped and size to be portable; however, this is not required. The lid 720 of the medical kit can include a compartment 722 that is designed to removably hold a tablet computer 724, smart device, smart phone, IPad, or the like. As can be appreciated, the lid of the medical kit can incorporate a permanent display. If a tablet computer, IPad, or the like is used with the container, the tablet computer, IPad, of the like is used as the display for the personal medical kiosk. The camera on the tablet computer, IPad, or the like can be used to convey images to the remotely located medical provider. Software can be loaded into the tablet computer, IPad, or the like to interface with the remotely located medical provider, and/or the medical kit can include hardware and/or software that is used with the tablet computer, IPad, or the like. The one or more medical devices included in the base can be wirelessly connected or directly to the hardware and/or software of the medical kit, and/or be wirelessly connected or directly to the tablet computer, IPad, or the like. The medical kit can include a lock or other security arrangement to inhibit or prevent unauthorized use of the medical kit. The medical kit can include one or more connectors (e.g., USB, Bluetooth, Wi-Fi, etc.).

It will thus be seen that the objects set forth above, among those made apparent from the preceding description, are efficiently attained, and since certain changes may be made in the constructions set forth without departing from the spirit and scope of the invention, it is intended that all matter contained in the above description and shown in the accompanying drawings shall be interpreted as illustrative and not in a limiting sense. The invention has been described with reference to preferred and alternate embodiments. Modifications and alterations will become apparent to those skilled in the art upon reading and understanding the detailed discussion of the invention provided herein. This invention is intended to include all such modifications and alterations insofar as they come within the scope of the present invention. It is also to be understood that the following claims are intended to cover all of the generic and specific features of the invention herein described and all statements of the scope of the invention, which, as a matter of language, might be said to fall therebetween. The invention has been described with reference to the preferred embodiments. These and other modifications of the preferred embodiments as well as other embodiments of the invention will be obvious from the disclosure herein, whereby the foregoing descriptive matter is to be interpreted merely as illustrative of the invention and not as a limitation. It is intended to include all such modifications and alterations insofar as they come within the scope of the appended claims.

I claim:

1. A medical kiosk having a modular configuration and designed to provide tele-medicine services to a user, said medical kiosk including a user video conferencing system, said user video conferencing system designed to enable the user to have a real-time or near real-time tele-conference with a medical provider located remotely from said medical kiosk, said user video conferencing system including a first screen, a camera, and a microphone, said first video screen designed to display said medical provider during a video conference between said user and said medical provider, said medical kiosk including a scheduling system that enables a patient to schedule an appointment with a medical provider.

2. The medical kiosk as defined in claim 1, including a medical kit that includes a plurality of medical devices, two or more of said medical devices selected from the group consisting of a stethoscope, an otoscope, a thermometer, a dermascope, a spirometer, blood pressure cuff, a pulse oximeter, a blood glucometer, an audiometer, and a 3-lead EKG.

3. The medical kiosk as defined in claim 2, wherein said medical kit includes a lid and a base, said base includes a plurality of compartments, at least two of said compartments each include a different medical device, said lid including a display that enables a user to view a remotely located medical provider.

4. The medical kiosk as defined in claim 3, wherein said display is in the form of a portable smart device that is removably connected to a cavity in said lid.

5. The medical kiosk as defined in claim 1, including a mobile device having a housing that includes a plurality of medical devices selected from the group consisting of a stethoscope, an otoscope, a thermometer, a dermascope, a spirometer, blood pressure cuff, a pulse oximeter, a blood glucometer, an audiometer, and a 3-lead EKG.

6. The medical kiosk as defined in claim 1, wherein one or more medical device panels are used to control and/or display results from one or more medical devices.

7. The medical kiosk as defined in claim 1, wherein a PHR can be formed or accessed prior to, during, or after a tele-medicine session in the medical kiosk.

8. The medical kiosk as defined in claim 1, including a mobile device application or network application, said mobile application or network application designed to allow a user various functions on a mobile device or computer, said functions including one or more functions selected from the group consisting of 1) locating an available medical kiosk, 2) scheduling an appointment with the medical kiosk, 3) pre-registering symptoms and/or reasons for visit, 4) set and/or cancel an appointment with the medical kiosk, 5) receiving reminders and/or updates regarding appointments for the medical kiosk, 6) obtaining information about medical kiosk availability, 7) obtaining information about certain medical provider availability, 8) obtaining information about the available medical provider, 9) enabling the selection of a certain medical provider and/or medical provider in a certain field of medicine, 10) obtaining map information, address information and/or hours of operation information regarding a selected medical kiosk, 11) locating the closest kiosk and/or kiosk availability for a certain medical provider and/or medical provider in a certain field of medicine, 12) presubmitting and/or preclearing medical insurance, 13) submitting payment information, 14) receiving information on payment status, 15) receiving information on insurance coverage, 16) receiving appointment reminders and/or updates, 17) receiving prescription information, 18) submitting payment information for medical visit and/or prescription, 19) answer surveys regarding the use of the medical kiosk, and 20) receiving medication reminders.

9. The medical kiosk as defined in claim 1, including a medication adherence software application that provides one or more functions selected from the group consisting of 1) enabling a patient to speak to a pharmacist, 2) enabling a patient to change the patient's medications alerts, 3) enabling a patient to learn about certain types of medical conditions, 4) enabling a patient to check medication orders status, 5) enabling a patient to check medication delivery status, 6) enabling a patient to refill a prescriptions, 7) enabling a patient to transfer prescriptions to a another location, 8) enabling a patient to obtain information about recommended medication dosages, 9) enabling a patient to obtain information about recommended times to take medications, 10) enabling a patient to obtain information about recommended frequency for taking medications, 11) enabling a patient to obtain information about medications, 12) enabling a patient to obtain information about generic brands available for medications, 13) enabling a patient to request an appointment to speak with a medical provider, 14) enabling a patient to enter information regarding compliance information regarding medication usage by patient, 15) enabling a patient to receive compliance reports for patients regarding medication usage, 16) enabling a patient to pay for the prescription, 17) enabling a patient to select between a generic and non-generic brand of medication, 18) enabling a patient to compare pricing and/or delivery options for various medications, 19) provides reminders for patients to take their medications, 20, monitors and/or generates progress reports to the patient regarding adherence to medication usage, and 21) enrolls a patient in an electronic prescriptions network which sends their prescriptions to their choice of pharmacy.

10. The medical kiosk as defined in claim 1, including an exterior registration station, said registration station including a user input system that enables the user to enter information about the user prior to having said real-time or near real-time teleconference with a medical provider, said user input system can include one or more components selected from the group consisting of a key pad for identification and/or data entry, keyboard for identification and/or data entry, a touch screen for identification and/or data entry, microphone and/or voice recognition software for identification and/or data entry, a fingerprint scanner for identification and/or data entry, a retina scanner for identification and/or entry, lights, buttons, switches, camera, speakers, microphone, scanner, receiver, transmitter, credit card/debit card or other some other card reader, smart phone or other smart device reader/scanner, finger and/or eye scanner, shelf, printer, storage cavity, service access door, motion sensor, sound sensor, temperature sensor, and logos, said registration station can be used for one or more functions selected from the group consisting of a) allowing patient to enter patient name, b) allowing patient to enter patient address, c) allowing patient to enter patient contact information, d) allowing patient to enter patient age, e) allowing patient to enter patient sex, f) allowing patient to enter patient height, g) allowing patient to enter patient weight, h) allowing patient to enter patient race, i) allowing patient to enter patient language, j) allowing patient to enter patient medical history, k) allowing patient to enter current and/or past medicines used by patient, l) allowing patient to enter information on past medical condition, m) allowing patient to enter reason(s) for visit by patient, n) allowing patient to enter patient current symptoms, o) allowing patient to enter patient's allergies, p) allowing patient to enter patient's ID number and/or password, q) allowing patient to enter patient insurance information, r) allowing patient to enter patient payment information, s) allowing patient to enter consent forms, t) allowing patient to request assistance from a medical attendant, u) allowing patient to enter date and/or time patient desires appointment, v) allowing patient to enter patient's current medical provider, w) allowing patient to request a specific medical provider and/or medical provider specialty, x) providing patient information about the medical kiosk, y) providing patient information on how to use the medial kiosk, z) providing patient information on how to properly input/convey information to the medical kiosk, aa) providing patient instructions and/or interactions with the patient during the inputting/conveying of information by the patient to the medical kiosk, bb) providing patient information on the wait time for the patient's use of the medical kiosk, cc) providing patient a list of patients and/or number of patients waiting to use the medical kiosk, and dd) providing patient information regarding whether the medical kiosk is in use or is available.

11. The medical kiosk as defined in claim 1, including a display screen that can be used to provide one or more types of information selected from the group consisting of registration information, information input by the user, advertising information, information about the medical kiosk, information about wait time for a medical kiosk, information as to the order of users waiting to use the medical kiosk, information about whether a medical kiosk is available or in use, cable TV shows, satellite TV shows, local broadcast TV shows, infomercials, medical programs, DVD materials, Blu-ray materials, video programs, YouTube programs, and pictures.

12. The medical kiosk as defined in claim 1, including an interior chamber and a cleaning or sanitizing system to clean or sanitize at least a portion of said interior chamber, said cleaning or sanitizing system including a system selected from the group consisting of a UV system and a mist system.

13. The medical kiosk as defined in claim 1, including a prescription drug system that includes one or more functions selected from the group consisting of 1) enabling a medical provider to generate electronic prescriptions, 2) enabling a patient to conveniently select and/or order prescription drugs, 3) enabling a patient to choose between name brand and generic drugs, 4) enabling a patient to choose the supply quantity for the prescription, 5) enabling a patient to choose between picking the prescription up at the pharmacy of their choice or mail delivery of the prescription, 6) enabling a patient to enter medical insurance for partial or full payment of the prescription, 7) enabling a patient to enter a credit or debit card information to pay for the prescription, 8) enabling a patient to enter information for mail delivery of the prescription, 9) enabling a patient to enter information to provide automatic reminders to patient regarding refilled and/or follow-up medical visits, 10) enabling a patient to enter information to enable patient to be notified when prescription has been mailed and/or is ready to be picked-up at the pharmacy, 11) enabling a patient to obtain a print out and/or electronic version of the prescription written by the medical provider, 12) enabling a patient to receive information about the issued prescription in printout and/or electronic form, and 13) enabling a patient to select the pharmacy where he/she would like to pick-up the prescription.

14. A method for providing a real-time or near real-time tele-conference with a medical provider located remotely from a medical kiosk and a patient located at the medical kiosk, said method comprising the steps of:
a. providing a medical kiosk;
b. providing an attendant application, an appointment application, a provider application, a schedule system, a patient portal, medical adherence application, an administrator application, and/or a provider portal, said attendant application includes software and/or hardware that enables the attendant to 1) monitor, modify and/or cancel existing appointments for a medical kiosk, 2) monitor whether a patient requires assistance, 3) monitor and/or assistant a patient during registration with the medical kiosk, 4) provide procedures and/or check lists for the medical assistant, and/or 5) provide information on the status of the medical kiosk, said attendant application can include one or more functions selected from the group consisting of a) displaying one or more appointment fields of a patient, said appointment fields can include one or more fields selected from the group consisting of time of appointment, patient name, medical provider name, status of appointment and action of attendant, b) checking-in a patient, c) cancelling and/or rescheduling an appointment, d) entering notes about a visit, e) entering notes about a canceled and/or rescheduled visit, f) viewing notes from a medical provider about a patient, g) printing a visit summary, h) allowing the medical attendant to associate an emergency clean-up with a patient appointment, i) allowing medical assistant to view appointment information, j) allowing medical assistant to validate patient IDs, k) allowing medical assistant to validate patient insurance, l) allowing medical assistant to reschedule or cancel an appointment, m) allowing medical assistant to perform the sanitization process for the medical kiosk, n) allowing medical assistant to select the patient records, o) allowing medical assistant to enter in and/or scan ID and/or insurance information, p) providing notice of instructions and/or policies to be given to a patient, q) allowing medical assistant to update patient information, r) allowing medical assistant to enter payment and/or copay information and/or payments for the patient visit, s) displaying a list of sanitation protocols and/or procedures, and t) allowing medical assistant to activate automatic sanitation process, said patient appointment application can include one or more functions selected from the group consisting of 1) enabling a patient to identify themselves as a new and/or returning patient, 2) enabling a patient to schedule a new appointment, verify a previously confirmed appointment and/or cancel an appointment, 3) welcoming a patient that has already scheduled an appointment, 4) providing the patient with a privacy policy associated with the use of the medical kiosk, 5) providing the patient with terms of service policy associated with the use of the medical kiosk, 6) requesting information as to why the patient is using the patient registration system, 7) requesting the patient to enter a password, phone number, pin number, name, email address, address, date or birth and/or sex to identify the patient, 8) requesting the patient to verify if the patient is at least a certain age, 9) requesting the patient to identify one or more symptoms that the patient is experiencing in which the patient is seeking medical assistance, 10) requesting the patient to answer one or more follow-up questions after the patient enters the one or more symptoms, 11) informing the patient to seek an in-person visit by a medical provider after the patient has identified one or more symptoms that the patient is experiencing, 12) requesting the patient to identify one or more known allergies of the patient, 13) requesting the patient to identify one or more known medical conditions of the patient, 14) requesting the patient to identify one or more past medical procedures that have been performed on the patient, 15) requesting the patient to identify one or more medications the patient is taking, 16) asking the patient if the patient has medical insurance, 17) requesting the patient to enter in the medical insurance information and/or to scan the insurance card, 18) requesting the patient to make a copay based the medical insurance or to fully pay for the medical visit, 19) allowing the patient to request assistance, 20) listing one or more days and/or times that the medical kiosk is available for use by the patient, 21) verifying all of the required information and optional payment has been made by the patient by displaying information on a screen, printing out verification for the patient, emailing verification to patient, texting verification to the patient and/or leaving a voicemail of verification for the patient, 22) allowing a patient to update information, 23) storing information in a patient file, 24) navigating the patient through the vitals capture process, 25) navigating the patient through the patient consultation, and 26) navigating the patient through a survey, said provider application is designed to have one or more features selected from the group consisting of a) enabling a medical provider display appointment information, b) enabling a medical provider display patient information, c) enabling a medical provider view and/or modify visit information, d) enabling a medical provider perform the consultation with the patient, and e) enabling a medical provider utilize one or more medical devices in the medical kiosk, said provider application can include one or more functions selected from the group consisting of 1) enabling the medical provider to view appointments that have been completed by the medical provider or are in progress for a certain day, 2) enabling the medical provider to view appointments that have been made for a future time, 3) enabling the medical provider to display one or more types of patient appointment information, said patient appointment information including information selected from the group consisting of time of appointment, patient names, status of appointment, patient started the registration process but has not yet been approved for payment, patient has been checked-in, appointment has started and vitals are being collected, vitals have been collected and the video consultation is in-progress, video consultation has been completed and medical provider completion is pending, and appointment has been completed, 4) enabling the medical provider to display a record about a patient, such record information selected from the group consisting of patient name, patient date of birth, patient sex, patient symptoms, patient medical conditions, date of last visit, diagnosis, allergies, medications, prior visit record of patient, and medical records of patient, 5) enabling the medical provider to notify the medical attendant that the patient requires assistance, 6) enabling the medical provider to limit access to a patient's information, 7) enabling the medical provider to refuse an appointment that has been created for the medical provider, 8) enabling the medical provider to begin the conference with the patient once the patient is ready in the medical kiosk, 9) enabling the medical provider to view the patient on the medical provider's screen, 10) enabling the medical provider to cause an end to the consultation with the patient, 11) allowing the medical provider to view the patient records, including the vitals capture process that has occurred or is occurring in the medical kiosk, 12) enabling the medical provider to modify a patient's record, 13) enabling the medical provider add and/or remove medication, medical conditions, allergies and/or symptoms information in the patient's record, 14) enabling the medical provider to enter notes about a patient, diagnoses for a patient, and/or follow-up care for a patient, 15) enabling the medical provider to control access to and/or activate one or more medical devices in the medical kiosk, 16) enabling the medical provider to view the results from the use of one or more medical devices that have been used by the patient, 17) enabling the medical provider to create a visit summary for a patient, 18) enabling the medical provider to create a prescription for a patient, 19) enabling the medical provider to provide one or more visit documents and/or other types of documents to the patient, 20) enabling the medical provide to contact another medical provide and optionally allow the primary medical provider to view the other medical provider on the provider screen and/or enable the other medical provider to be viewed by the patient, 21) enabling the medical provider to engage a translator to facilitate communication between the patient and medical provider, and 22) enabling the medical provider to contact a third party to obtain information about and/or authorization for a patient using the medical kiosk, said scheduling system can include one or more functions selected from the group consisting of 1) enabling a patient to select a particular medical provider, 2) making an appointment on a medical kiosk at a particular time and place, 3) enabling a patient to select a medical provider or type of medical provider based on the particular need of the patient, 4) locating available locations of medical kiosks, 5) enabling a patient to enter information about the patient, 6) checking past, current and/or future appointments of the patient regarding use of the medical kiosk, 7) entering a partial or full payment for use of the medical kiosk, 8) entering medical insurance information, 9) making payment information, 10) enabling a patient to request assistance, 11) enabling a patient to enter consents and/or consent forms, and 12) enabling the patient to receive reminders about the appointment, said patient portal including one or more functions selected from the group consisting of 1) allowing a patient to locate an available medical kiosk, 2) allowing a patient to schedule an appointment with the medical kiosk, 3) allowing a patient to pre-register symptoms and/or reasons for visit, 4) allowing a patient to set and/or cancel an appointment with the medical kiosk, 5) allowing a patient to receive reminders and/or updates regarding appointments for the medical kiosk, 6) allowing a patient to obtain information about medical kiosk availability, 7) allowing a patient to obtain information about certain medical provider availability, 8) allowing a patient to obtain information about the available medical provider, 9) allowing a patient to enable the selection of a certain medical provider and/or medical provider in a certain field of medicine, 10) allowing a patient to obtain map information, address information and/or hours of operation information regarding a selected medical kiosk, 11) allowing a patient to locate the closest kiosk and/or kiosk availability for a certain medical provider and/or medical provider in a certain field of medicine, 12) allowing a patient to presubmit and/or preclear medical insurance, 13) allowing a patient to submit payment information, 14) allowing a patient to receive information on payment status, 15) allowing a patient to receive information on insurance coverage, 16) allowing a patient to receive appointment reminders and/or updates, 17) allowing a patient to receive prescription information, 18) allowing a patient to submit payment information for medical visit and/or prescription, 19) allowing a patient to answer surveys regarding the use of the medical kiosk, and 20) allowing a patient to receive medication reminders, said medical adherence application is designed to reduce medication errors, said medical adherence application including one or more functions selected from the group consisting of 1) reminding patients to take their meds, 2) automatically creating a log when the patient takes their medication, 3) tracking a patient's compliance to taking their medication on time, 4) providing automatic progress reports, 4) referring the patient to a pharmacist to answer any questions and/or for additional consultation, 5) automatically enrolling the patient in a e-script network, which network sends their prescriptions to the pharmacy of their choice, 6) assisting in improving patient outcomes and/or satisfaction, 7) being designed to be used on a screen in the medical kiosk, and/or on a computer screen and/or mobile device, 8) allowing a patient to speak to a pharmacist, 9) allowing a patient to change medication alerts 9) providing information to a patient about various medical condition, 10) allowing the patient to check medication order status, 11) allowing the patient to refill prescriptions, 12) allowing patient to transfer prescriptions to a pharmacy, 13) providing patient with information about recommended dosages of medication, 14) providing information to patient regarding period for taking medications, 15) providing patient with information regarding frequency for taking medications, 16) providing information to patient regarding information about medications, 17) providing information to patient regarding generic brands available for medications, 18) allowing a patient to request an appointment to speak with a medical provider, 19) allowing the patient to enter compliance information regarding medication usage by patient, 20) providing patient with medication alerts, 21) allowing the patient to change medication alerts, 22) allowing the patient to select how information will be sent to patient, and 23) providing the patient with medication guidelines and/or medical plans, said an administrator application allowing a medical administrator to remotely access one or more components of the medical kiosk, said administrator application including one or more functions selected from the group consisting of 1) allowing remote access to one or more medical devices in the medical kiosk, 2) allowing remote access to one or more computers in the medical kiosk, 3) allowing remote access to one or more routers in the medical kiosk, 4) allowing remote access to one or more displays on the medical kiosk, 5) allowing diagnostics to be executed from a remote location on one or more electronic components in the medical kiosk, 6) allowing the rebooting and/or reinitializing from a remote location of one or more electronic components in the medical kiosk, 7) allowing for review of the current and/or past status from a remote location of one or more electronic components in the medical kiosk, 8) allowing remote access to one or more power supplies in the medical kiosk, 9) allowing remote access to one or more servers in the medical kiosk, 10) allowing remote access to one or more hard drives in the medical kiosk, 11) allowing hardware and/or software updates to be remotely sent and/or loaded onto one or more electronic components in the medical kiosk, and 12) allowing software to be loaded onto and/or removed from one or more electronic components in the medical kiosk, said provider portal can include one or more functions selected from the group consisting of 1) allowing a medical provider to access another medical provider, 2) enabling two or more medical providers to be simultaneously viewed and/or heard by the patient in the medical kiosk, 3) enabling the medical provider to switch to a new medical provider such that only the new medical provider is not viewed by the patient in the medical kiosk, and 4) allowing a second medical provider to listen and/or communicate with the patient in the medical kiosk.

15. The method as defined in claim 14, wherein said medical kiosk has a modular configuration, said medical kiosk having a user video conferencing system, said user video conferencing system designed to enable the user to have a real-time or near real-time tele-conference with a medical provider located remotely from said medical kiosk, said user video conferencing system including a first and second video screens, a camera, and a microphone, said first video screen designed to display said medical provider during a video conference between said user and said medical provider, said second video screen being a touch screen that enables said user to enter information regarding said tele-medicine services.

16. The method as defined in claim 14, including a mobile device application or network application, said mobile application or network application designed to allow a user various functions on a mobile device or computer, said functions including one or more functions selected from the group consisting of 1) locating an available medical kiosk, 2) scheduling an appointment with the medical kiosk, 3) pre-registering symptoms and/or reasons for visit, 4) setting and/or cancelling an appointment with the medical kiosk, 5) receiving reminders and/or updates regarding appointments for the medical kiosk, 6) obtaining information about medical kiosk availability, 7) obtaining information about certain medical provider availability, 8) obtaining information about the available medical provider, 9) enabling the selection of a certain medical provider and/or medical provider in a certain field of medicine, 10) obtaining map information, address information and/or hours of operation information regarding a selected medical kiosk, 11) locating the closest kiosk and/or kiosk availability for a certain medical provider and/or medical provider in a certain field of medicine, 12) presubmitting and/or preclearing medical insurance, 13) submitting payment information, 14) receiving information on payment status, 15) receiving information on insurance coverage, 16) receiving appointment reminders and/or updates, 17) receiving prescription information, 18) submitting payment information for medical visit and/or prescription, 19) answer surveys regarding the use of the medical kiosk, and 20) receiving medication reminders.

17. The method as defined in claim 14, wherein said medical kiosk includes a cleaning or sanitizing system to clean or sanitize at least a portion of an interior chamber, said cleaning or sanitizing system including a system selected from the group consisting of a UV system and a mist system.

18. The method as defined in claim 14, including a medical kit that includes a plurality of medical devices, two or more of said medical devices selected from the group consisting of a stethoscope, an otoscope, a thermometer, a dermascope, a spirometer, blood pressure cuff, a pulse oximeter, a blood glucometer, an audiometer, and a 3-lead EKG.

19. The method as defined in claim 18, wherein said medical kit includes a lid and a base, said base includes a plurality of compartments, at least two of said compartments each include a different medical device, said lid including a display that enables a user to view a remotely located medical provider, said display is in the form of a portable smart device that is removabley connected to a cavity in said lid.

20. The method as defined in claim 14, including a mobile device having a housing that includes a plurality of medical devices selected from the group consisting of a stethoscope, an otoscope, a thermometer, a dermascope, a spirometer, blood pressure cuff, a pulse oximeter, a blood glucometer, an audiometer, and a 3-lead EKG.

21. The method as defined in claim 14, wherein one or more medical device panels are used to control and/or display results from one or more medical devices.

22. The method as defined in claim 14, wherein a PHR can be formed or accessed prior to, during or after a tele-medicine session in the medical kiosk.
